(19) United States
(12) Patent Application Publication
(10) Pub. No.: US 2020/0276230 A1
(43) Pub. Date: Sep. 3, 2020

(54) PARTICLES FOR THE TREATMENT OF CANCER IN COMBINATION WITH RADIOTHERAPY

(71) Applicant: Xerion Healthcare Limited, Thame Oxfordshire (GB)

(72) Inventors: Matthew Ronald Phillip Stock, Brackley Northamptonshire (GB); Martin Ryan Gardener, Banbury Oxfordshire (GB); Gareth Wakefield, Oxford Oxfordhire (GB)

(73) Assignee: Xerion Healthcare Limited, Thame Oxfordshire (GB)

(21) Appl. No.: 16/764,350

(22) PCT Filed: Nov. 16, 2018
(86) PCT No.: PCT/GB2018/053330

§ 371 Date: May 14, 2020

(30) Foreign Application Priority Data
Nov. 17, 2017 (GB) 1719076.0

(51) Int. Cl. A61K 33/244; A61K 9/50; A61K 9/00; A61K 33/24; A61K 51/02

(52) U.S. Cl. CPC A61K 33/244; A61K 9/501; A61K 9/5026; A61K 33/24; A61K 51/025; A61K 9/0019

(57) ABSTRACT

The invention provides a particle comprising a first semiconductor and a second semiconductor wherein the first semiconductor forms a heterojunction with the second semiconductor. The invention also provides a pharmaceutical composition comprising the particles, and relates to uses of the particles and composition in the treatment of cancer in combination with radiotherapy.

### FIELD OF THE INVENTION

The invention relates to a particle and a pharmaceutical composition comprising a plurality of particles. The invention further relates to the use of the particle or pharmaceutical composition in the treatment of cancer in combination with radiotherapy.

### BACKGROUND TO THE INVENTION

Cancer is a class of diseases characterised by uncontrolled cell division. There are over 200 types of cancer which can develop within the body. As there are different types of cells within each organ there are multiple types of cancer that can develop at any given site. Cancer harms the body when damaged cells divide uncontrollably and form solid lumps called tumours which interfere with body functions and hormone levels as they grow. Tumours become much more difficult to treat once they undergo metastasis; the process when a cancer cell moves throughout the body using the blood or lymph systems, invades healthy tissue and begins to divide and grow to form a new tumour. One critical aspect of tumour structure is the presence of oxygen deficient, or hypoxic, regions which form as a result of blood vessel growth being slower than cellular division. These dormant regions are indicative of poor prognosis as they contain cells that are most resistant to either natural or treatment-induced cell death.

Radiotherapy is a key treatment for cancer, being used in approximately 50% of cancer treatments in the developed world. Radiotherapy can be used to cure cancer. It is estimated that radiotherapy is primary treatment modality used in 16% of patients who are cured of their cancer. By comparison, chemotherapy is the primary modality in only 2% of cancer cures.

Radiotherapy relies for its efficacy on the production of reactive oxygen species (ROS), also known as free radicals. Free radicals are highly reactive chemical species containing oxygen which can act to destroy cellular components such as DNA and membranes. Enough free radical damage will induce apoptosis, or cell death. Radiotherapy can be given in two different ways: from outside the body (known as external beam radiotherapy or external radiotherapy) or from inside the body (known as internal radiotherapy).

### External Radiotherapy

External beam radiotherapy works by targeting a beam of radiation, typically X-rays (high energy photons), at the tumour site. In some instances, beams of protons or electrons can be used.

X-rays may interact with the tumour cell either directly, direct absorption causing DNA damage, or indirectly as the incident X-ray scatters off, usually water, molecules in the tumour. Such scattering results in >90% of the incident X-ray energy being deposited in an electron. This highly energetic electron scatters off other nearby electrons causing a cascade effect where a field of progressively less energetic electrons are formed, resulting in generation of superoxide free radicals as the final de-excitation and consequent free radical induced cell damage. Molecular oxygen is required to form superoxide free radicals; consequently radiotherapy is less effective in hypoxic tumour regions. Of course damage to normal cells will also occur as a result of incident X-rays, consequently the radiation beam is carefully targeted and shaped to get as much of the energy into the tumour as possible. Whilst normal cells can repair themselves more efficiently against free radical damage than cancer cells there is not much difference in the ability of cancerous and normal tissue to absorb X-rays. Maximum radiotherapy dose is determined by the tolerance of the surrounding normal tissues, rather than the dose required to control tumour growth. In principle it is possible to cure any tumour with radiotherapy but in practice this would mean intolerable damage to patient's normal tissues as too high a dose would be required. The maximum dose that can be applied is typically 70-74Gy.

There is therefore what is known as a ‘therapeutic window’ for radiotherapy treatment. Too little radiation will have no effect whereas too much can lead to serious side effects such as damage to vital organs or radiation burns. Complicating this is the fact that various tumour types respond differently to radiotherapy, being more or less radiosensitive or radioresistant, as do various other organs in the body. Radiosensitive organs include salivary glands, liver, stomach, and others. These organs will typically withstand up to 35Gy of radiotherapy—too little for curative treatment of tumours in these or nearby organs.

The aim of all radiotherapy is to concentrate as much energy within the tumour region as possible whilst minimising exposure to normal tissue. Conventionally, this has been accomplished using a single external beam with the patient being exposed from several directions such as front and back or side to side. Although the technology is very well established it is limited in its ability to spare normal tissue from excessive radiation doses. Recent developments have included stereotactic radiosurgery (SRS) in which highly focused beams are used to target well defined tumour regions typically in the brain or spine. It is claimed that the ability to accurately target tumour regions and use shorter treatment regimes enhances the treatment efficacy. A typical example of a SRS system is Cyberknife™, which has had FDA clearance for treatment of tumours in any part of the body since 2001. The radiotherapy source is mounted on a robot arm and can deliver a pencil thin beam of radiation at 6-8Gy per minute. Again, the main rationale for this approach is to increase the dose accuracy to the tumour and deliver dose escalation. Intensity modulated radiation therapy (IMRT) utilises multiple radiation beams to deliver maximum energy into fields that accurately map even complex tumour structures such as those wrapping around blood vessels. One downside is that experienced medical professionals are required to map the structure one image at a time prior to devising a treatment protocol. However, there is increasing evidence of advanced survival using both SRS and IMRT techniques and reduced toxicity and normal tissue damage.

Proton therapy uses an external beam of protons to target the tumour site, the advantage being an ability to target a tumour mass more easily than using X-ray radiotherapy. This is due to the protons having limited side scatter due to their high mass and a well-defined penetration depth. In a similar fashion to X-ray based treatments the protons may either directly damage DNA by scattering or indirectly by free radical generation. It is unclear at present whether this technique leads to an overall survival advantage in cancer treatment although off target toxicity reduction has been demonstrated.

Electron beam radiotherapy employs a medical linear accelerator to generate electrons, which are then directed to the desired area. For superficial tumours, electron beam radiotherapy may be used directly on the skin. For deeper tumours intraoperative electron radiotherapy (IOERT) may be used.

### Internal Radiotherapy—Brachytherapy, SIRT and Radiopharmaceuticals

Brachytherapy is an internal radiotherapy in which radioactive seeds are implanted within the tumour mass. It is commonly used for prostate, cervical, breast and skin cancer. The radioactive sources emit primarily gamma radiation (high energy photons) or beta radiation (high energy electrons). These particles then create reactive oxygen species (ROS) which act to destroy cancer cells in much the same way as external beam radiotherapy. However, the radiation is not strong enough for treatment of aggressive tumours as the radiation range is low; it is generally used for low grade tumours of the prostate and can cause urinary and erectile problems.

Selective internal radiation therapy (SIRT) beads contain a radioactive isotope, usually yttrium-90, encased in a glass or polymer bead. This is injected into the blood vessels close to the tumour, the beads then block these blood vessels and the radiation, usually β or γ rays, acts to destroy tumour cells in the same way as external beam radiotherapy.

Radiopharmaceuticals are a group of drugs which are radioactive which are principally used in the treatment of bone metastases. Key actives are 223-Ra dichloride (Xofigo)— an alpha (α-He nucleus) emitter which is injected intravenously, and 153-Sm ethylenediaminetetramethylenephosphonic acid (EDTMP) (Quadramet)—a beta (β-650, 710, 810 keV) and gamma (γ-103 keV) emitting radionuclide. These drugs work in a similar way to all internal radiotherapy treatments in that the primary treatment route is the generation of free radicals following photon or particle interaction with outer shell electrons.

### Augmenting the Effects of Radiotherapy

There have been many attempts over the past six decades to enhance the therapeutic effects of radiotherapy, whilst minimising normal tissue damage, by using inorganic materials, usually nanoparticles, directly injected in to the tumour.

Gold and other high atomic mass nanoparticles have been most commonly employed. Such high atomic number (high-Z) nanoparticles enhance the scattering of X-ray generated photoelectrons, effectively slowing them down so they get to the final free radical generating event with a shorter path length. This has the overall effect of making the free radical generation region resulting from any given X-ray scattering event within the tumour smaller. Since the overall energy dissipated is equal to the free radical concentration within this volume is increased and the effectiveness of cancer cell killing per incident X-ray is increased. However, they are limited by the requirement of oxygen to be present in the tumour since the final de-excitation event is the formation of a superoxide free radical.

US2009186060 A1 describes the use of 0.5-400 nm gold nanoparticles as radiotherapy enhancers in the treatment of cancer. The nanoparticles showed efficacy in increasing the lifetime of breast cancer carrying mice, albeit at high nanoparticle loadings.

WO2009147214 describes the use of high molecular weight (density of >7 g·cm³) metal oxide, principally hafhium oxide, nanoparticles as radiotherapy enhancers in a similar way to gold. Radiotherapy enhancement is demonstrated on a number of cell lines, again at high nanoparticle load. In a further publication, Maril et al. (Radiation Oncology 2014, 9:150) describe clonogenic cancer cell assays using hafhium oxide nanoparticles in a variety of cell lines. In one example, radioresistant pancreatic cancer cells (Panc-1) show a radiotherapy dose enhancement factor (DEF) of 1.3 when 800 μM of hafhium oxide nanoparticles are combined with radiotherapy. Radiotherapy dose enhancement factor (DEF) is defined as: DEF=[Dose with Radiation Alone/Dose with Radiation+active material] for the same biological effect. If the DEF is greater than one, then the addition of the drug is functioning as a radiosensitiser. If the DEF is less than one, then the drug is a radioprotector. Typically, DEF is measured using a clonogenic cell assay at 90% cell death.

WO2011070324 describes a different approach to nanoparticle augmented radiotherapy. Titanium oxide nanoparticles are used as a host lattice for rare earth dopant elements. Titanium oxide is a photoactive semiconductor material that directly generates free radicals under radiotherapy when doped with small amounts of rare earth ions. Rare earth ions act effectively to scatter photogenerated electrons, transferring the energy to the titanium oxide host lattice which generates free radicals with a quantum efficiency of 8-10%. This approach allows nanoparticles to act as ‘hot spots’ of free radical generation within cancer cells and enhances cell killing over that of high-Z nanoparticles. One particular advantage in the use of photoactive inorganics is the ability to generate free radicals via the hole in the valance band of the semiconductor. This mechanism does not require oxygen, since the free radical generation now involves water splitting into hydroxyl free radicals rather than the generation of superoxides. This allows more effective targeting of the most dangerous hypoxic tumour regions. It is limited by the low amount of high-Z elements that can be doped into the lattice and the relatively low efficiency of free radical generation following particle excitation.

US20170000887A1 describes a nanoparticle device based on a phosphor core and a photoactive titanium oxide shell. The core contains a wide band gap (8 to 9 eV) insulator material, NaYF₄, which acts as a host for optically active rare earth ions. The rare earth ions upconvert infrared photons to visible and/or ultraviolet photons which act to excite a titanium oxide shell. The excited shell will then de-excite, creating free radicals which are used to induce cancer cell apoptosis. This nanoparticle-augmented photodynamic therapy is limited by the requirement to excite the particles using a fibre optic cable to administer the near-infrared light; it can only treat cancer where the cable can reach and light can penetrate. It does not have the ability to target any solid tumour in the way that radiotherapy does.

EP 2187445 relates to a material comprising an array of nanoparticles for use in photovoltaic cells. The nanoparticle array alters between bridge and core structures in order to create localised states for exciton generation and delocalised states for carrier extraction.

WO 2013/019090 relates to hydrophilic nanoparticles that are used in magnetic resonance imaging as a contrast agent.

US 2016/0022976 relates to a method for hyperthermal treatment of tumour cells using nanoparticles. The particles are designed to heat up under an applied alternating magnetic field to destroy tumour cells.

Ways to increase free radical generation from titanium oxide nanoparticles have been explored in other, non-medical fields, for example in the context of water purification and biocides. One way to accomplish this is to use a semiconductor:metal heterojunction structure. In this approach metals such as silver are used to decorate the surface of titanium oxide nanoparticles. Ultraviolet light excites the titanium oxide. Following excitation the band structure of the device allows electrons to migrate to the silver surface cluster while the holes remain localised in the titanium oxide core. This physical separation of the charges reduces the probability of recombination across the titanium oxide band gap and enhances the generation of free radicals using the electrons localised on the surface silver clusters. US20140183141 A1 describes such an approach using a photocatalyst containing titanium oxide and silver surface clusters (see FIG. 14 in the publication for the energy band diagram) in the form of solid composites formed from glass bubbles and cement binder. The composites are designed for use in water purification. Whilst more effective at generating free radicals than titanium oxide alone, the composites and the approach described in US20140183141 A1 would not help in the treatment of cancer since they rely on ultraviolet light rather than radiotherapy and on using electrons in the presence of oxygen to generate free radicals. Ultraviolet light cannot penetrate into the human body to any significant depth and therefore, unlike radiotherapy, is unsuitable for treating tumours. Also, the fact that the approach relies on the presence of oxygen to generate free radicals means that it would not in any case be effective for hypoxic (low oxygen) regions of tumours.

### SUMMARY OF THE INVENTION

The present invention recognises and deals with a particular limitation of conventional radiotherapy and other known particles used in cancer treatment. In particular, in order for the therapy to be effective, an adequate level of molecular oxygen needs to be present in the cancerous tissue being treated. The limitation arises because conventional radiotherapy techniques of the kind discussed hereinbefore rely on energetic incident electrons, generated in vivo from the radiation, being able to react with molecular oxygen at the site of the cancer in order to produce superoxide radicals. The superoxide radicals act to destroy nearby cancer cells, by overwhelming the cells' antioxidant defence capacity. The concentration of molecular oxygen during irradiation is therefore critical in determining subsequent biological response, meaning that the efficacy of radiotherapy is significantly greater for well-oxygenated cells and tissue.

The present invention recognises that the reliance of the therapy on the presence of molecular oxygen is a significant limitation, given that cancerous tumours are generally known to contain a substantial fraction of cells which are hypoxic. The invention addresses this issue by providing access to an alternative mechanism by which reactive oxygen species (ROS) can be generated in vivo by radiotherapy, which does not require the presence of molecular oxygen, and which therefore circumvents the need for molecular oxygen. As will be discussed further below, the invention achieves this by providing a radiosensitising particle, suitable for use in combination with radiotherapy, which facilitates the generation of ROS directly from water, and irrespective of the level or presence of molecular oxygen at the site of the cancer, by the following valence band hole-mediated water-splitting reaction:

h⁺+H₂O→H⁺OH^{•}

Furthermore, in contrast to known particles which are lattice-doped with a high-Z element, particles of the present invention exhibit enhanced photoactivity because the amount of a second, typically high-Z element present in the particles of the invention is greater than can be achieved using simple lattice doping. This further increases interaction with X-rays and photogenerated electrons, allowing for higher-efficiency free radical generation. Therefore, radiotherapy efficacy is increased, allowing for a more effective treatment of deep solid tumours than has hitherto been demonstrated using known particles. This permits energy to be concentrated in a tumour site, and permits the use of less energy overall, thus allowing for better treatment of, for example, radiosensitive organs. Radiosensitive organs will typically only withstand up to 35Gy of radiotherapy—too little for curative treatment of tumours in these or nearby organs.

The current invention achieves the enhanced radiotherapy by employing a semiconductor heterojunction, comprising a first semiconductor in contact with a second semiconductor, to generate free radicals. The energy bands of the two phases are aligned such that holes are migrated to the second semiconductor and electrons are localised within the first semiconductor. This charge splitting is further improved by having semiconductors with bands lined up such that the electron affinity of the second semiconductor is smaller than the electron affinity of the first semiconductor, and the energy difference from the top of the valance band to vacuum level is smaller in the second semiconductor than in the first semiconductor (in other words, the top of the valence band, V_{b}², of the second semiconductor is at a higher energy than the top of the valence band, V_{b}¹, of the first semiconductor: V_{b}¹<V_{b}²). This arrangement aids the separation of the electron and the hole, thus minimising radiative recombination and improving efficiency of free radical generation.

A staggered (Type II) heterojunction (see FIGS. 2, 3 and 7) is often preferred because it more effectively splits charge and minimises charge recombination. The critical parameters are the electronic band gap, E_{g}, (energy gap between conduction and valance bands, often referred to simply as the “band gap”) and electron affinity, E_{A} (energy difference between vacuum level and bottom of the conduction band). In a staggered (Type II) heterojunction between two semiconductors, the first semiconductor forming the junction has a greater electron affinity than the second semiconductor (E_{A}¹>E_{A}²). Secondly, the top of the valence band, V_{b}¹, of the first semiconductor is at a lower energy than the top of the valence band, V_{b}², of the second semiconductor (V_{b}¹<V_{b}²). In terms of E_{A} and E_{g}, this means that the sum of the electron affinity and the electronic band gap for the first semiconductor (E_{A}¹+E_{g}¹) is greater than the sum of the electron affinity and the electronic band gap for the second semiconductor (E_{A}²+E_{g}²), so that E_{A}¹+E_{g}¹>E_{A}²+E_{g}². Thirdly, the top of the valence band of the second semiconductor is at a lower energy than the bottom of the conduction band, C_{b}¹, of the first semiconductor (V_{b}²<C_{b}¹). In terms of E_{A} and E_{g}, this means that the sum of the electron affinity and the electronic band gap for the second semiconductor (E_{A}²+E_{g}²) is greater than the electron affinity of the first semiconductor (E_{A}¹); in other words, E_{A}²+E_{g}²>E_{A}¹.

When ionising radiation is directed at the particle, the incident energy will eject an electron, e⁻, from a deep electronic level (resulting in a free electron which may go on to interact with other nearby particles) leaving behind a hole, h⁺, in the deep electronic level. Electrons of higher energy within the solid will drop into the hole level resulting in migration of the hole to the top of the valence band, V_{b}. It is also possible that incident energy will act to promote an electron into the conduction band, C_{b}, of the material. This is likely to occur to a greater extent following interaction of the particle with electrons generated as a result of scattering with other particles, since the energy of the incident electrons will be lower and less likely to promote ionisation. These interactions will result in electrons populating the conduction bands, C_{b}, and holes populating the valance bands, V_{b}. If a single semiconductor contains both electrons and holes there is a high probability that they will recombine radiatively with emission of a photon of energy equivalent to the band gap. The particles of the present invention, on the other hand, minimise charge recombination and optimise water-splitting, by providing a heterojunction that facilitates splitting of the electrons and holes into separate regions of the particle—into the first and second semiconductors thereof—in order to maximise the potential for de-excitation via water splitting, and minimise radiative recombination. This can be mediated either via the electron, as:

e⁻+O₂→^{•}O₂⁻

or the hole as:

h⁺+H₂O→H⁺+OH^{•}

In order for the splitting of water to proceed, it must be energetically favourable in that energy must be lost by transitions of electrons from the conduction band to the oxygen level and by holes to the water level. Materials suitable for the particles can be assessed using the scientific literature where both calculated and experimentally-measured band gaps and electron affinities are well documented. Zhai H J and Wang L S, J. Am. Chem. Soc 129 (2007) 3022-3026 describe a method of measuring titanium oxide band structures using ultraviolet photoelectron spectroscopy. Stevanovic V et al, Phys. Chem. Chem. Phys. 16 (2014) 3706-3714 describe calculations for a variety of semiconductor materials, including titanium oxide, in relation to water oxidation and reduction energy levels. Lanthanide oxide electron structures, both calculated and experimental, have been collated by Gillen R et al, Phys. Rev. B 87 (2013) 125116. Once formed, superoxide and hydroxyl free radicals may be used to damage cellular components.

Holes generate free radicals by water splitting and may therefore be used irrespective of the oxygen level of the tumour regions, i.e. hypoxic regions may be targeted. The present invention recognises that the reliance of known therapies on the presence of molecular oxygen is a significant limitation, given that cancerous tumours are generally known to contain a substantial fraction of cells which are hypoxic. Once generated, the hydroxyl free radicals act in a similar manner to superoxide radicals, to destroy nearby cancer cells by overwhelming the cells' antioxidant defence capacity. Hydroxyl radicals are believed to oxidize the membrane lipids of cells to produce peroxidants, which then set up a series of peroxidant chain reactions; the oxidatively stressed malignant cells progress to a necrotic state that results in their destruction.

In this way, the particles of the invention may be employed in combination with radiotherapy to remove the reliance on molecular oxygen at the site of the cancer, and increase the efficacy of the radiotherapy in hypoxic environments.

Accordingly, in a first aspect, the invention provides a particle comprising a first semiconductor and a second semiconductor wherein the first semiconductor forms a heterojunction with the second semiconductor.

Often, in the particle of the invention, the heterojunction is a staggered heterojunction. Thus, the first and second semiconductors are often chosen such that a staggered (Type II) heterojunction is formed at the interface between the two semiconductors. In other words, the first and second semiconductors are typically chosen such that (i) E_{A}¹>E_{A}², (ii) E_{A}¹+E_{g}¹>E_{A}²+E_{g}², and (iii) E_{A}²+E_{g}²>E_{A}¹, wherein E_{A}¹ and E_{A}² are the respective electron affinities of the first and second semiconductors and E_{g}¹ and E_{g}² are the respective electronic band gaps of the first and second semiconductors.

The invention further provides a pharmaceutical composition comprising (i) a plurality of particles of the invention, wherein each of the particles comprises a first semiconductor and a second semiconductor wherein the first semiconductor forms a heterojunction with the second semiconductor, and optionally (ii) one or more pharmaceutically acceptable ingredients.

The invention additionally provides a particle of the invention as defined above, or a pharmaceutical composition of the invention as defined above, for use in the treatment of the human or animal body by therapy.

The invention further provides a particle of the invention as defined above, or a pharmaceutical composition of the invention as defined above, for use in combination with radiotherapy in the treatment of cancer in a subject.

The invention also provides a method of treating cancer in a subject, the method comprising administering to a subject a particle of the invention as defined above, or a pharmaceutical composition of the invention as defined above, and performing radiotherapy on the subject.

The invention further provides the use of a particle of the invention as defined above in the manufacture of a medicament for use in combination with radiotherapy in the treatment of cancer.

The invention also provides the use of a pharmaceutical composition of the invention as defined above in the manufacture of a medicament for use in combination with radiotherapy in the treatment of cancer.

The invention further provides a kit of parts comprising:

a plurality of particles, wherein each of said particles comprises a first semiconductor and a second semiconductor, wherein the first semiconductor forms a heterojunction with the second semiconductor; and

instructions for the use of the particles, in combination with radiation from an external source or from a radioactive material inside the subject, for the treatment of cancer in a subject.

The invention also provides a kit of parts comprising:

a plurality of particles, wherein each of said particles comprises a first semiconductor and a second semiconductor, wherein the first semiconductor forms a heterojunction with the second semiconductor;

a radioactive material suitable for internal radiation therapy, and

optionally, instructions for the use of the particles, in combination with radiation from said radioactive material, for the treatment of cancer in a subject.

The present invention also provides an in vitro method of destroying cancer cells, which method comprises: contacting a particle of the invention as defined above or a pharmaceutical composition of the invention as defined above, with a composition comprising cancer cells, and directing ionising radiation at the cancer cells.

The present invention also provides a process for producing free radicals, the process comprising exposing a particle of the invention as defined above to ionising radiation in the presence of water.

The present invention also provides a particle of the invention as defined above, or a pharmaceutical composition of the invention as defined above, for use in a diagnostic method practiced on the human or animal body.

The invention further provides the use of a particle of the invention as defined above, or the use of a pharmaceutical composition of the invention as defined above, for determining the presence or absence of cancer.

The invention also provides a method for determining the presence or absence of cancer comprising administering to a subject a particle of the invention as defined above, or a pharmaceutical composition of the invention as defined above, and detecting the presence or absence of the particle of the invention, or particles of the invention, at a site suspected of being cancerous.

### BRIEF DESCRIPTION OF THE FIGURES

FIG. 1 is a schematic illustration of one preferred structure for the particle of the invention, comprising (i) a core of titanium dioxide, (ii) surface regions disposed on the core comprising a wide band gap, high molecular mass semiconductor which is in contact with the titanium dioxide to form heterojunctions with the titanium dioxide at the interfaces (regions of contact) between the two materials, and (iii) an optional silica coating, disposed on the outer surface of the particle (i.e. on the surface of the titanium dioxide, in regions where the titanium dioxide is outermost, and on the surface of the wide band gap, high molecular mass semiconductor, in regions where that semiconductor is outermost).

FIG. 2 is a schematic illustration of the mechanism of action when a particle of the invention (comprising a heterojunction between TiO₂ and Gd₂O₃ and an outer coating of silica) situated within a solid tumour is exposed to [A] high-energy photons (for example X-rays, γ) or high-energy particles (for example electrons, e⁻, or protons, p⁺) from external or internal radiotherapy. [B] shows the incident high-energy particle or photons interact by scattering within a deep electronic level within the Gd₂O₃ phase, creating a hole or electron-hole pair in the Gd₂O₃ phase. Similarly, [C] shows the incident high-energy particle or photons interact with a deep electronic level within the TiO₂ phase, creating a hole or electron-hole pair in the TiO₂ phase. [D] shows photogenerated electrons being scattered from the particle as a result. [E] shows a hole migrating from the TiO₂ phase to the top of the valance band in the Gd₂O₃ phase. [F] shows a hole from the valance band of the Gd₂O₃ phase quantum tunneling through the silica coating and splitting water at the surface of the particle, to create a hydroxyl free radical. [G] shows an electron migrating from the Gd₂O₃ conduction band to the conduction band in the TiO₂ phase, which reduces charge recombination. [H] shows an electron still present in the Gd₂O₃ conduction band quantum tunneling through the silica coating and forming a superoxide free radical in combination with any molecular oxygen that may be present at the surface of the particle.

FIG. 3 is a schematic illustration of the mechanism of action when a particle of the invention (comprising a heterojunction between TiO₂ and Lu₂O₃ and an outer coating of silica) situated within a solid tumour is exposed to [A] high-energy photons (for example X-rays, γ) or high-energy particles (for example electrons, e⁻, or protons, p⁺) from external or internal radiotherapy. [B] shows the incident high-energy particle or photons interact by scattering within a deep electronic level within the Lu₂O₃ phase, creating a hole or electron-hole pair in the Lu₂O₃ phase. Similarly, [C] shows the incident high-energy particle or photons interact with a deep electronic level within the TiO₂ phase, creating a hole or electron-hole pair in the TiO₂ phase. [D] shows photogenerated electrons being scattered from the particle as a result. [E] shows a hole migrating from the TiO₂ phase to the top of the valance band in the Lu₂O₃ phase. [F] shows a hole from the valance band of the Lu₂O₃ phase quantum tunneling through the silica coating and splitting water at the surface of the particle, to create a hydroxyl free radical. [G] shows an electron migrating from the Lu₂O₃ conduction band to the conduction band in the TiO₂ phase, which reduces charge recombination. [H] shows an electron still present in the Lu₂O₃ conduction band quantum tunneling through the silica coating and forming a superoxide free radical in combination with any molecular oxygen that may be present at the surface of the particle.

FIG. 4 shows two electron micrographs of two different particles, both formed from TiO₂ and Lu₂O₃ in a 0.91:0.09 mass ratio. The micrographs show a plurality of Lu₂O₃ semiconductor regions disposed on TiO₂.

FIG. 5 is a table showing the composition of particles formed from TiO₂ and Lu₂O₃ with various amounts of Lu by mass. Lu amounts from 2.1 wt. % to 9.5 wt. % were measured by energy dispersive X-ray analysis (EDX) and X-ray photoelectron spectroscopy (XPS). EDX measures the bulk overall lutetium concentration, XPS measures the Lu from a surface region of up to 10 nm deep. Separation of Lu₂O₃ into a surface phase is indicated by an increase in the XPS signal in comparison to the EDX signal.

FIG. 6 is a graph showing pancreatic cancer (Panc-1) cell survival in units of % (y-axis) versus the X-ray dose in units of Grays (x-axis) of the radiotherapy for (i) radiotherapy alone with no particles (dashed line), versus (ii) particle-augmented radiotherapy using particles of titanium dioxide doped with a rare-earth element as described in WO2011070324 (dotted and dashed line), and (iii) particle-augmented radiotherapy using particles of the invention (“semiconductor device enhanced radiotherapy”) formed from TiO₂ and Lu₂O₃ in a 0.91:0.09 mass ratio (solid line). The particles of the invention (iii) resulted in a Dose Enhancement Factor (DEF) of 1.9 at a concentration of 57 μM per well. A rare earth doped particle (ii) at the same concentration gave a DEF of only 1.24.

FIG. 7 is a schematic illustration of the three types of semiconductor heterojunctions organised by band alignment. Electron affinity (E_{A}) and band gap (E_{g}) are shown for the Type II (staggered) heterojunction. Type II staggered heterojunctions split charges in the valance and conduction bands into the separate semiconductor phases.

FIG. 8 is an electron micrograph image of a heterojunction particle with a 2.5 nm amorphous silica coating (shown with an arrow).

FIG. 9 is a transmission electron micrograph of nanoparticles containing TiO₂ and Lu₂O₃ in a 0.91:0.09 mass ratio produced in accordance with Example 5.

FIG. 10 is a table showing the dose enhancement factors (DEF) measured by a pancreatic cancer (PANC-1) clonogenic assay between 0 and 3 Gy radiotherapy for rare earth nanoparticles in accordance with Example 8.

FIG. 11 is a graph showing the results of an in vivo Mia-PaCa2 xenograft trial demonstrating delay in tumour growth following addition of a nanoparticle formulation as described in Example 9. The formulation shows 2.5 times the effectiveness of tumour control compared with radiotherapy alone.

FIG. 12 is a graph showing the results of an in vivo radioresistant colorectal xenograft trial demonstrating delay in tumour growth following addition of a nanoparticle formulation as described in Example 12. The formulation shows 8.1 times the effectiveness of tumour control compared with radiotherapy alone (tumour volume doubling time).

FIG. 13 is a schematic illustration of the mechanism of action when a particle of the invention (comprising a heterojunction between TiO₂ and Yb₂O₃) situated within a solid tumour is exposed to [A] high-energy photons (for example X-rays, γ) or high-energy particles (for example electrons, e⁻, or protons, p⁺) from external or internal radiotherapy. [B] shows the incident high-energy particle or photons interacting by scattering within a deep electronic level within the Yb₂O₃ phase, creating a hole or electron-hole pair in the Yb₂O₃ phase. Similarly, [C] shows the incident high-energy particle or photons interacting with a deep electronic level within the TiO₂ phase, creating a hole or electron-hole pair in the TiO₂ phase. [D] shows photogenerated electrons being scattered from the particle as a result. [E] shows a hole migrating from the TiO₂ phase to the top of the valance band in the Yb₂O₃ phase. [F] shows a hole from the valance band of the Yb₂O₃ phase splitting water at the surface of the particle to create a hydroxyl free radical. [G] shows an electron migrating from the Yb₂O₃ conduction band to the conduction band in the TiO₂ phase, which reduces charge recombination. [H] shows an electron still present in the Yb₂O₃ conduction band forming a superoxide free radical in combination with any molecular oxygen that may be present at the surface of the particle. Electronic properties of Yb₂O₃ are given in Witorczyk T and Wesolowska A, Physica Status Solidi A, Vol. 82, K67 (1984) and Prokofiev A V, Shelykh A I and Melekh B T, Journal of Alloys and Compounds, Vol. 242, 41 (1996).

FIG. 14 is a transmission electron micrograph of nanoparticles containing TiO₂ and Gd₂O₃ in a 0.93:0.07 mass ratio produced in accordance with Example 6.

FIG. 15 is a transmission electron micrograph of nanoparticles containing TiO₂ and Yb₂O₃ in a 0.93:0.07 mass ratio produced in accordance with Example 7.

### DETAILED DESCRIPTION OF THE INVENTION

The invention relates to a particle comprising a first semiconductor and a second semiconductor wherein the first semiconductor forms a heterojunction with the second semiconductor.

The term “semiconductor” as used herein refers to a material with electrical conductivity intermediate in magnitude between that of a conductor and a dielectric. The term “semiconductor” as used herein, does not therefore include materials with a band gap of equal to or greater than 6.0 eV, it being understood that such materials are generally dielectric materials, i.e. insulators or very poor conductors of electric current. The first and second semiconductors employed in the particle of the invention therefore each have a band gap of less than 6.0 eV.

The skilled person is readily able to measure the band gap of a semiconductor, by using well-known procedures which do not require undue experimentation, and the band gaps of many semiconductors are known in the art. The band gap of titanium dioxide for instance, is known to be about 3.2 eV, and the band gaps of lutetium oxide (Lu₂O₃) and gadolinium oxide (Gd₂O₃) are known to be about 5.5 eV and about 5.4 eV respectively. The band gap of a semiconductor may be estimated by constructing a photovoltaic diode or solar cell from the semiconductor and determining the photovoltaic action spectrum. The monochromatic photon energy at which the photocurrent starts to be generated by the diode can be taken as the band gap of the semiconductor; such a method was used by Barkhouse et al., Prog. Photovolt: Res. Appl. 2012; 20:6-11. Furthermore, Zhai H J and Wang L S, J. Am. Chem. Soc 129 (2007) 3022-3026 describe a method of measuring titanium oxide band structures using ultraviolet photoelectron spectroscopy, and Lanthanide oxide electron structures, both calculated and experimental, have been collated by Gillen R et al, Phys. Rev. B 87 (2013) 125116. Table II of Gillen et al. lists the following experimentally-determined band gaps for the lanthanide sesquioxides, citing Prokofiev A. Shelykh, and B. Melekh, Journal of Alloys and Compounds 242, 41 (1996): La₂O₃=5.5 eV; Ce₂O₃=2.4 eV; Pr₂O₃=3.9 eV; Nd₂O₃=4.7 eV; Sm₂O₃=5 eV; Eu₂O₃=4.4 eV; Gd₂O₃=5.4 eV; Tb₂O₃=3.8 eV; Dy₂O₃=4.9 eV; Ho₂O₃=5.1 eV; Er₂O₃=5.3 eV; Tm₂O₃=5.4 eV; Yb₂O₃=4.9 eV; and Lu₂O₃ 5.5 eV.

The term “heterojunction”, as used herein, takes its normal meaning in the art, referring to the interface that occurs between two regions of different semiconductors. The semiconductors generally have unequal (different) band gaps, in contrast to a homojunction. The heterojunction in the particle of the invention is a heterojunction between the first semiconductor and the second semiconductor.

The first and second semiconductors exist in the particle as two distinct phases. The first semiconductor forms the heterojunction with the second semiconductor at a point of contact between the two phases. The particle may comprise: (i) a first region comprising (or consisting of) the first semiconductor, and (ii) a second region comprising (or consisting of) the second semiconductor, wherein the second region is disposed on the surface of the first region. Alternatively, the particle may comprise: (i) a first region comprising (or consisting of) the second semiconductor, and (ii) a second region comprising (or consisting of) the first semiconductor, wherein the second region is disposed on the surface of the first region. In such embodiments, the second region typically forms said heterojunction with the first region. The first region may for instance be a central “core” of the particle. The first region is typically therefore a core. The first region often comprises (or consists of) the first semiconductor. The second region then comprises (or consists of) the second semiconductor.

As discussed above, the first and second semiconductors exist in the particle as two distinct phases and the first semiconductor may form the heterojunction with the second semiconductor at a point of contact between the two phases. There may however be more than one point of contact between the first semiconductor and the second semiconductor. For instance, the particle may comprise: (i) a first region comprising (or consisting of) the first semiconductor, and (ii) a plurality of second regions, each comprising (or each consisting of) the second semiconductor and each of which is disposed on the surface of the first region. Alternatively, the particle may comprise: (i) a first region comprising (or consisting of) the second semiconductor, and (ii) a plurality of second regions, each comprising (or each consisting of) the first semiconductor and each of which is disposed on the surface of the first region. In such embodiment, each second region may form a heterojunction with the first region. The particle may therefore comprise a plurality of heterojunctions. The first region may for instance be a central “core” of the particle. The first region often comprises (or consists of) the first semiconductor. The second regions then comprise (or consist of) the second semiconductor.

The first and second semiconductors are different semiconductor materials, i.e. they comprise different semiconductor compounds.

The particle may consist essentially of the first and second semiconductors. The particle may for instance consist of (i.e. consist only of) the first and second semiconductors. Often, however, the particle comprises further materials in addition to the first and second semiconductors. It may, for instance, further comprise a coating. Suitable coatings are discussed further hereinbelow.

The particle of the invention may be a nanoparticle or a microparticle.

The term “nanoparticle”, as used herein, means a microscopic particle whose size is typically measured in nanometres (nm). A nanoparticle typically has a particle size of from 0.1 nm to 500 nm, for instance from 0.5 nm to 500 nm. A nanoparticle may for instance be a particle having size of from 0.1 nm to 300 nm, or for example from 0.5 nm to 300 nm. Often, a nanoparticle has a particle size of from 0.1 nm to 100 nm, for instance from 0.5 nm to 100 nm.

The term “microparticle”, as used herein, means a microscopic particle whose size is typically measured in micrometres (μm). A microparticle usually has a particle size of greater than 0.1 μm, and more typically has a particle size of greater than 0.5 μm. The particle size of a microparticle is typically up to 500 μm. Often, however, a microparticle has a particle size of up to 100 μm. A microparticle may for instance have a particle size of from greater than 0.1 μm to 500 μm, for instance from 0.5 μm to 500 μm, or from greater than 0.5 μm to 500 μm. For instance, a microparticle may have a particle size of from greater than 0.1 μm to 100 μm. A microparticle may for example be a particle having a particle size of from greater than 0.5 μm to 100 μm.

A particle, for instance a nanoparticle or a microparticle, may have a high sphericity, i.e. it may be substantially spherical or spherical. A particle with a high sphericity may for instance have a sphericity of from 0.8 to 1.0. The sphericity may be calculated as π⅓(6Vₚ)⅔/A_{P} where V is the volume of the particle and Aₚ is the area of the particle. Perfectly spherical particles have a sphericity of 1.0. All other particles have a sphericity of lower than 1.0.

A particle may alternatively be non-spherical. It may for instance be in the form of an oblate or prolate spheroid, and it may have a smooth surface. Alternatively, a non-spherical particle may be plate-shaped, needle-shaped, tubular or take an irregular shape.

Microparticles which have a high sphericity, i.e. are substantially spherical are referred to herein as “microspheres”. A plurality of such microparticles, for instance a plurality of the radioactive embolization particles described herein, may have an average (mean) sphericity of from 0.8 to 1.0.

Typically, the first semiconductor has a higher electron affinity than the second semiconductor (E_{A}¹>E_{A}²).

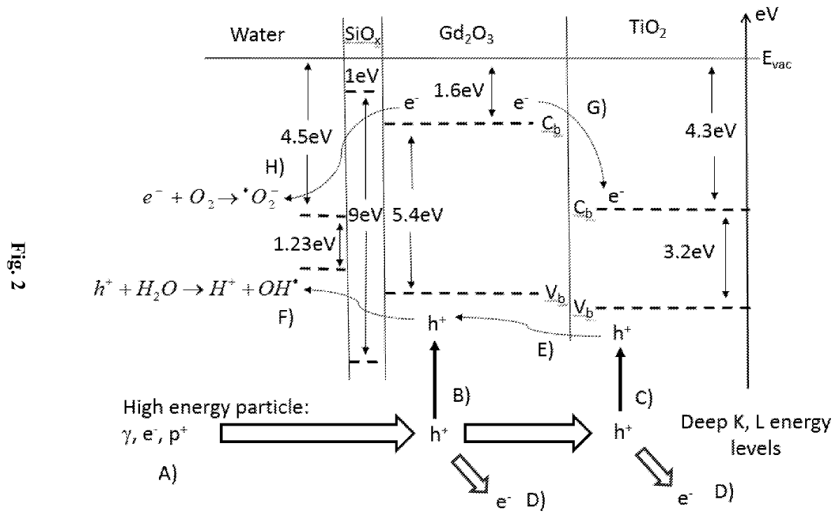

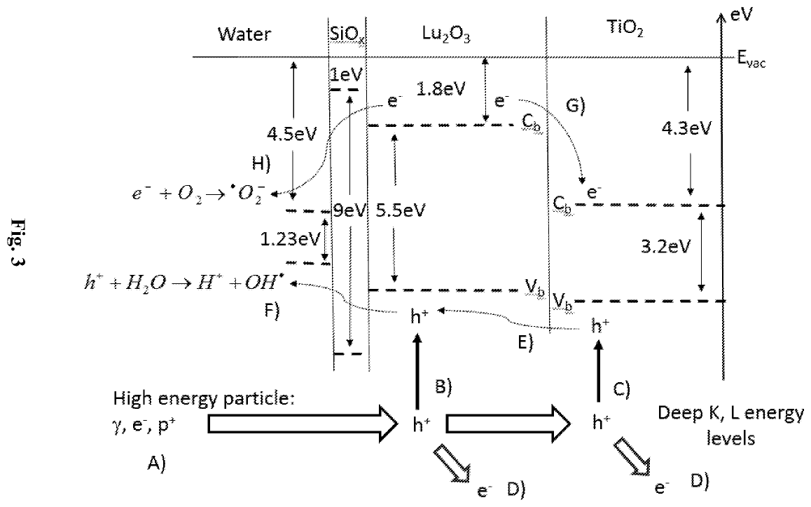

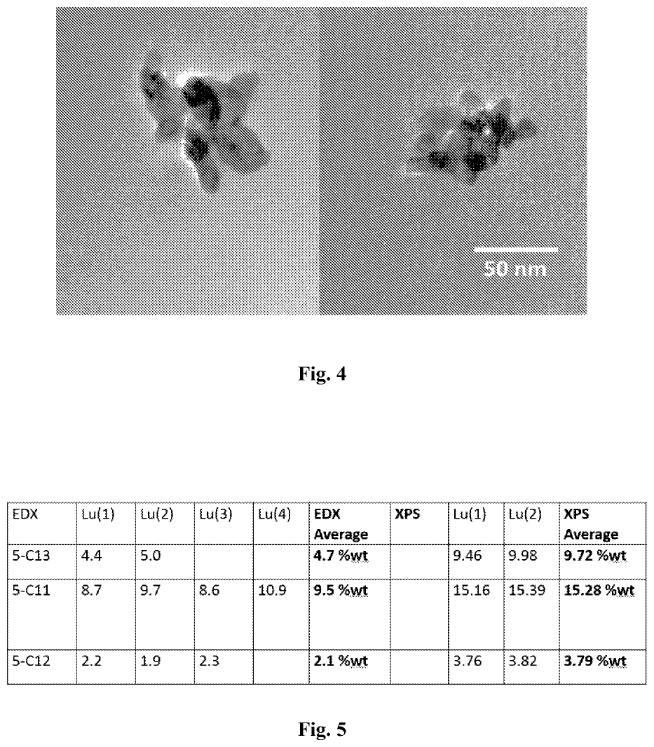

The term “electron affinity” (E_{A}) as used herein refers to the energy obtained by moving an electron from the vacuum to the bottom of the conduction band. The skilled person is readily able to measure the electron affinity of a material, by using well-known procedures which do not require undue experimentation, and the electron affinities of many semiconductors are known in the art. The electron affinity of titanium dioxide for instance, is known to be about 4.3 eV. The water energy levels on the left hand side of FIGS. 2 and 3 are given in Stevanovic 2014, Phys. Chem. Chem. Phys., 2014, 16, 3706. The corresponding relative figures for Titanium Oxide are given in FIG. 4 in Chapter 8 ‘Advancement of Sol-Gel prepared TiO₂ photocatalyst’ in ‘Recent Applications in Sol-Gel Synthesis’ 2017 published by Intech. The TiO₂ electron affinity can also be found in Solar Materials Science, Edited by Laurence E Merr, Academic Press, 2012 Page 641. The electron affinities of lutetium oxide (Lu₂O₃) and gadolinium oxide (Gd₂O₃) are about 1.8 eV and about 1.6 eV respectively. The electron affinities can be derived from conduction band offset measurements of electronic devices and the corresponding work function of base layer. In Peredo M et al, Surface and Interface Analysis, 38, 494 (2006) the conduction band offset of Lu₂O₃ on Ge is measured as 2.2 eV. Tallej N, Surface Science, 69, 428 (1977) gives the work function of Ge as 4 eV. This gives the corresponding electron affinity of Lu₂O₃ of 1.8 eV. Chu L K et al, Applied Physics Letters, 94, 202108 (2009) give the conduction band offset of Gd₂O₃ on Ge as 2.4 eV, giving a Gd₂O₃ electron affinity of 1.6 eV. The electron affinity of a material, e.g. a semiconductor, may readily be measured using ultraviolet photoelectron spectroscopy, as described, for example in Photoelectron Spectroscopy-Principles and Applications, Stefan Hufher, 3^{rd} revised edition 2003. The difference in electron affinities encourages electrons in the conduction band to migrate towards the semiconductor of higher electron affinity, thus enhancing separation of the electron in the conduction band and the hole in the valence band, thereby minimising electron-hole recombination and allowing more efficient free radical generation.

Typically, the top of the valence band of the first semiconductor is at a lower energy than the top of the valence band of the second semiconductor (V_{b}¹<V_{b}²). In terms of E_{A} and E_{g}, this means that the sum of the electron affinity and the electronic band gap for the first semiconductor (E_{A}¹+E_{g}¹) is greater than the sum of the electron affinity and the electronic band gap for the second semiconductor (E_{A}²+E_{g}²), so that E_{A}¹+E_{g}¹>E_{A}²+E_{g}². The “sum of the electron affinity and the electronic band gap” herein refers to the sum of the magnitudes of these two energies (so that, if the electron affinity were expressed as a negative number, and the electronic band gap as a positive number, the negative sign of the electron affinity would be ignored).

The top of the valence band of the first semiconductor being at a lower energy than the top of the valence band of the second semiconductor causes holes to migrate into the valence band of the second semiconductor by encouraging electrons to migrate to the top of the valence band of lower energy in the first semiconductor, thus enhancing separation of the electron in the conduction band and the corresponding hole in the valence band, thereby minimising electron-hole recombination and allowing more efficient free radical generation.

Typically, the heterojunction is a staggered (Type II) heterojunction. A staggered (Type II) heterojunction is shown schematically in FIG. 7, the critical parameters being the electronic band gap, E_{g}, and the electron affinity, E_{A}. As discussed hereinbefore, in a Type II staggered heterojunction between two semiconductors, the first semiconductor forming the junction has a greater electron affinity than the second semiconductor (E_{A}¹>E_{A}²). Secondly, the top of the valence band, V_{b}¹, of the first semiconductor is at a lower energy than the top of the valence band, V_{b}², of the second semiconductor (V_{b}¹<V_{b}²). In terms of E_{A} and E_{g}, this means that the sum of the electron affinity and the electronic band gap for the first semiconductor (E_{A}¹+E_{g}¹) is greater than the sum of the electron affinity and the electronic band gap for the second semiconductor (E_{A}²+E_{g}²), so that E_{A}¹+E_{g}¹>E_{A}²+E_{g}². Thirdly, the top of the valence band of the second semiconductor is at a lower energy than the bottom of the conduction band, C_{b}¹, of the first semiconductor (V_{b}²<C_{b}¹). In terms of E_{A} and E_{g}, this means that the sum of the electron affinity and the electronic band gap for the second semiconductor (E_{A}²+E_{g}²) is greater than the electron affinity of the first semiconductor (E_{A}¹); in other words, E_{A}²+E_{g}²>E_{A}¹. As shown in FIGS. 2 and 3, this will lead to the formation of a Type II staggered semiconductor heterojunction that acts effectively to split charges.

The first and second semiconductors are typically therefore chosen such that the conditions discussed above for a staggered semiconductor heterojunction to be formed are satisfied. Thus, the first and second semiconductors are typically therefore chosen such that (i) E_{A}¹>E_{A}², (ii) E_{A}¹+E_{g}¹>E_{A}²+E_{g}², and (iii) E_{A}²+E_{g}²>E_{A}¹. A staggered (Type II) heterojunction may then be formed at the or each interface between the two semiconductors. The band gap (E_{g}) electron affinity (E_{A}) of any given semiconductor can readily be measured by the skilled person. Furthermore, the electron affinities and band gaps of many semiconductors are already known in the art. The skilled person can readily therefore determine whether or not any two given semiconductors would form a staggered heterojunction by reference to the literature or experimentally without undue burden.

The first semiconductor may for instance form a plurality of staggered (Type II) heterojunctions with the second semiconductor. There may for instance be more than one point of contact in the particle between the first semiconductor and the second semiconductor. For instance, the particle may comprise: (i) a first region consisting of the first semiconductor, and (ii) a plurality of second regions, each consisting of the second semiconductor and each of which is disposed on the surface of the first region. Alternatively, the particle may comprise: (i) a first region consisting of the second semiconductor, and (ii) a plurality of second regions, each consisting of the first semiconductor and each of which is disposed on the surface of the first region. In such embodiments, each second region may form a heterojunction with the first region, and the particle may therefore comprise a plurality of heterojunctions. The first region may for instance be a central “core” of the particle. The first region, which may be a core, often consists of the first semiconductor. In such embodiments, each of the second regions typically forms a staggered (Type II) heterojunction with the first region, and the particle typically therefore comprises a plurality of staggered (Type II) heterojunctions.

When ionising radiation is directed at the particle, the incident energy will eject an electron, e⁻, from a deep electronic level (resulting in a free electron which may go on to interact with other nearby particles) and leaving behind a hole, h⁺, in the deep electronic level. Electrons of higher energy within the solid will drop into the hole level resulting in migration of the hole to the top of the valence band, V_{b}. It is also possible that incident energy will act to promote an electron into the conduction band, C_{b}, of the material. This is likely to occur to a greater extent following interaction of the particle with electrons generated as a result of scattering with other particles, since the energy of the incident electrons will be lower and less likely to promote ionisation. These interactions will result in electrons populating the conduction bands, C_{b}, and holes populating the valance bands, V_{b}. If a single semiconductor contains both electrons populating the conduction band, C_{b}, and holes populating the valance band, V_{b}. there is a high probability that they will recombine radiatively with emission of a photon of energy equivalent to the band gap. However, the present invention minimises this to a great extent by providing a heterojunction—typically a staggered (Type II) heterojunction, or a plurality of staggered (Type II) heterojunctions—in the particle that facilitates splitting of the electrons and holes into separate regions of the particle—into the first and second semiconductors thereof—in order to minimise radiative combination of the electrons with the holes and to maximise de-excitation via water splitting.

Typically, the particle comprises a core comprising one of the semiconductors. The core may comprise, consist essentially of, or consist of the first semiconductor. The core typically consists of the first semiconductor. In other embodiments, however, the core may comprise, consist essentially of or consist of the second semiconductor. The term “core” as used herein generally refers to the body of the particle, as opposed to a shell or a coating. Typically, the term “core” refers to the central, innermost part of the particle.

The core may be coated, for instance partially coated, with the other of the two semiconductors.

The particle may therefore comprise: (i) a core which comprises the first semiconductor, and (ii) a region disposed on the surface of the core which comprises the second semiconductor. The core may consist of the first semiconductor and the region disposed on the surface of the core may consist of the second semiconductor. The region which is disposed on the surface of the core may completely envelope the core, i.e. it may fully coat the core. Usually, however, it does not fully coat the core. Thus, usually, the region which is disposed on the surface of the core is disposed on only part of the surface of the core. Typically, therefore, the surface of the core in this embodiment has a region which is not coated with the second semiconductor.

Alternatively, the particle may comprise: (i) a core which comprises the second semiconductor, and (ii) a region disposed on the surface of the core which comprises the first semiconductor. The core may consist of the second semiconductor and the region disposed on the surface of the core may consist of the first semiconductor. The region which is disposed on the surface of the core may completely envelope the core, i.e. it may fully coat the core. Usually, however, it does not fully coat the core. Thus, usually, the region which is disposed on the surface of the core is disposed on only part of the surface of the core. Typically, therefore, the surface of the core in this embodiment has a region which is not coated with the first semiconductor.

Typically, however, the core comprises (or consists of) the first semiconductor.

The particle of the invention may comprise: (i) a core which comprises the first semiconductor, and (ii) a plurality of regions disposed on the surface of the core, each of which comprises the second semiconductor. The core may consist of the first semiconductor and the plurality of regions disposed on the surface of the core may consist of the second semiconductor. Each of the regions disposed on the surface of the core may form a said heterojunction with the core. The plurality of regions which are disposed on the surface of the core usually do not fully coat the core. Rather, usually, the plurality of regions which are disposed on the surface of the core only partially coat the surface of the core. Typically, therefore, the surface of the core in this embodiment has one or more regions which are not coated with the second semiconductor.

Alternatively, the particle of the invention may comprise: (i) a core which comprises the second semiconductor, and (ii) a plurality of regions disposed on the surface of the core, each of which comprises the first semiconductor. The core may consist of the second semiconductor and the plurality of regions disposed on the surface of the core may consist of the first semiconductor. Each of the regions disposed on the surface of the core may form a said heterojunction with the core. The plurality of regions which are disposed on the surface of the core usually do not fully coat the core. Rather, usually, the plurality of regions which are disposed on the surface of the core only partially coat the surface of the core. Typically, therefore, the surface of the core in this embodiment has one or more regions which are not coated with the first semiconductor.

As mentioned above the core may be partially coated with the other of the two semiconductors. For instance, the particle may comprise a core comprising the first semiconductor wherein the core is partially coated with the second semiconductor. The particle may alternatively comprise a core comprising the second semiconductor, wherein the core is partially coated with the first semiconductor.

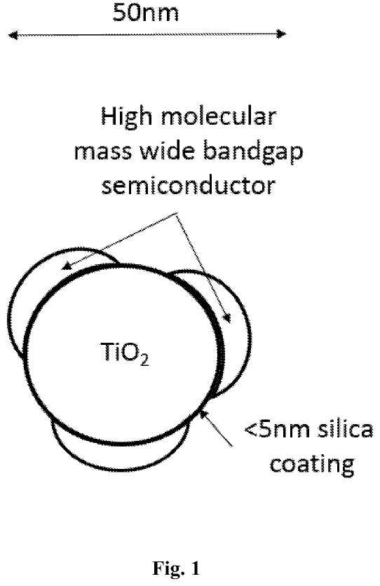

Accordingly, one semiconductor may be disposed on a surface of the other semiconductor such that portions of both the first and second semiconductors are exposed. This is illustrated in FIG. 1, which shows schematically the core of the material, typically comprising titanium dioxide, with portions of the second semiconductor typically comprising a lanthanide oxide, on the surface. The core of the first semiconductor is not completely coated with the second semiconductor: portions of both the first and second semiconductors in the particle are exposed to the surrounding environment.

In such embodiments of the particle of the invention both the first and second semiconductors are able to interact with the outside environment to generate free radicals, either through direct contact, or via a thin coating material (such as silica, alumina or a polymer such as a polyphosphate) as described hereinbelow.

Thus, in the context of the invention, “exposed” means either exposed to the outside environment directly (e.g. to the environment of a tumour or other cancerous site to which the particle has been administered) or via an outer coating material of the particle (such as silica, alumina or a polymer such as a polyphosphate) as described hereinbelow.

As explained above, the two semiconductors in the particle act to separate the electron and hole. By having both semiconductors exposed, this arrangement readily permits both holes and electrons generated in the particle to interact with the surrounding environment. Generation of free radicals can be mediated either via the electron, as:

e⁻+O₂→^{•}O₂⁻

or via the hole as:

h⁺+H₂O→H⁺+OH^{•}

and by having both semiconductors exposed, both processes are able to occur simultaneously.

One critical aspect of tumour structure is the presence of oxygen deficient, or hypoxic, regions which form as a result of blood vessel growth being slower than cellular division. These dormant regions are indicative of poor prognosis as they contain cells that are most resistant to either natural or treatment-induced cell death. In particular, the action of holes generated in the particle on water allow the particle to generate free radicals in the absence of oxygen, e.g. in hypoxic tumour regions, thus allowing the particle to target cells more resistant to conventional treatments.

The second semiconductor should preferentially, therefore, be the outermost of the structure since this is where holes are localised following excitation by X-rays. Thus, typically the particle comprises a core of a first semiconductor partially coated with the second semiconductor, such that portions of both the first and second semiconductors are exposed.

Often, therefore, the particle comprises (i) a core which comprises the first semiconductor, and (ii) a region disposed on part of the surface of the core which comprises the second semiconductor. The surface of the core therefore has an area which is not coated with said region. Portions of both the first and second semiconductors will therefore be exposed. The core may consist of the first semiconductor and the region disposed on part of the surface of the core may consist of the second semiconductor. The surface of the core therefore has an area which is not coated with the second semiconductor.

The particle may for instance comprise (i) a core which comprises the first semiconductor, and (ii) a plurality of regions disposed on the surface of the core, each of which comprises the second semiconductor, wherein the surface of the core has one or more areas which are not coated with said regions. Portions of both the first and second semiconductors will therefore be exposed. The core may consist of the first semiconductor and the plurality of regions disposed on the surface of the core may consist of the second semiconductor, wherein the surface of the core has one or more areas which are not coated with the second semiconductor.

Generally, the first semiconductor comprises a compound of a first metal, and the second semiconductor comprises a compound of a second metal. The compound of the first metal may for instance be an oxide of the first metal. Similarly, the compound of the second metal may be an oxide of the second metal. Thus, the first semiconductor may comprise an oxide of a first metal, and the second semiconductor may comprise an oxide of a second metal.

The second metal typically has a higher atomic number (Z) than the first metal.

The first metal and the second metal may be independently selected from the rare earth elements, the transition metals or the p-block metals. These classes of metals are discussed further hereinbelow.

The term “atomic number” or “Z” as used herein refers to the number of protons in the nucleus of the atom.

The second semiconductor typically comprises a high-Z semiconductor oxide. The presence of the high-Z phase results in a high level of interaction with X-rays and photogenerated electrons allowing deep tumours to be targeted. In addition to enhanced photoactivity, the amount of high-Z element present is generally greater than can be achieved simply by lattice doping, further increasing the interaction with X-rays and photogenerated electrons compared to what could be achieved using prior art systems. Radiotherapy efficacy is thereby increased, allowing for a more effective treatment of deep solid tumours than has hitherto been demonstrated using inorganic nanoparticles.

Thus, the atomic number (Z) of the first metal may, for instance, be 50 or less. The atomic number (Z) of the second metal may, for instance, be more than 50. Therefore, the second semiconductor usually has a higher molecular mass than the first semiconductor. In some cases, the atomic number (Z) of the first metal may be 45 or less, or 40 or less, or 35 or less or 30 or less. The atomic number (Z) of the second metal may be 55 or more. For instance, the first metal may have an atomic number of 45 or less and the second metal may have an atomic number of 50 or more. The first metal may have an atomic number of 40 or less and the second metal may have an atomic number of 50 or more. The first metal may have an atomic number of 35 or less and the second metal may have an atomic number of 50 or more. The first metal may have an atomic number of 30 or less and the second metal may have an atomic number of 50 or more. In some cases, the first metal may have an atomic number of 50 or less and the second metal may have an atomic number of 55 or more. The first metal may have an atomic number of 45 or less and the second metal may have an atomic number of 55 or more. The first metal may have an atomic number of 40 or less and the second metal may have an atomic number of 55 or more. The first metal may have an atomic number of 35 or less and the second metal may have an atomic number of 55 or more. The first metal may have an atomic number of 30 or less and the second metal may have an atomic number of 55 or more.

Typically, the first metal is a transition metal having an atomic number (Z) of 50 or less.

For instance, the first metal may be selected from scandium, yttrium, titanium, zirconium, vanadium, niobium, chromium, molybdenum, manganese, technetium, iron, ruthenium, cobalt, rhodium, nickel, palladium, copper, silver, zinc or cadmium (Sc, Y, Ti, Zr, V, Nb, Cr, Mo, Mn, Tc, Fe, Ru, Co, Rh, Ni, Pd, Cu, Ag, Zn or Cd). Usually, the first metal is Ti.

Typically, the second metal is selected from a lanthanide, hafnium (Hf), zirconium (Zr), tungsten (W) or tantalum (Ta). More usually, the second metal is a lanthanide, i.e. the second metal is selected from lanthanum, cerium, praseodymium, neodymium, promethium, samarium, europium, gadolinium, terbium, dysprosium, holmium, erbium, thulium, ytterbium or lutetium (La, Ce, Pr, Nd, Pm, Sm, Eu, Gd, Tb, Dy, Ho, Er, Tm, Yb or Lu). All of the isotopes of promethium (Pm) are radioactive. It is therefore preferred that the rare earth element is selected from La, Ce, Pr, Nd, Sm, Eu, Gd, Tb, Dy, Ho, Er, Tm, Yb and Lu. The rare earth element may be selected from Ce, Pr, Nd, Sm, Eu, Tb, Dy, Ho, Er, Tm, Yb and Lu. The rare earth element may for instance be Lu, Yb or Gd, for example it may be Lu. The rare earth element may be Gd. The rare earth element may be Yb.

Often, the second metal is selected from Lu, Yb and Gd.

The second metal may for instance be Lu.

The second metal may for example be Gd.

The second metal may for instance be Yb.

The term “transition metal” as used herein means any one of the three series of elements arising from the filling of the 3d, 4d and 5d shells, and situated in the periodic table following the alkaline earth metals. This definition is used in N. N. Greenwood and A. Earnshaw “Chemistry of the Elements”, First Edition 1984, Pergamon Press Ltd., at page 1060, first paragraph, with respect to the term “transition element”. The same definition is used herein for the term “transition metal”. Thus, the term “transition metal”, as used herein, includes all of Sc, Y, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd and Hg. These are also referred to as the first, second and third row transition metals (i.e. the transition metals in periods 4, 5 and 6 of the periodic table).

The term “p-block metal” as used herein means any metal in the p-block of the periodic table. Thus, the term “p-block metal”, as used herein, refers to a metal selected from Al, Ga, In, Tl, Sn, Pb and Bi.

The terms “lanthanide” and “rare earth element”, as used herein, take their normal meaning in the art, meaning any one of the fifteen metallic chemical elements with atomic numbers 57 through 71, from lanthanum through lutetium, i.e. any one of La, Ce, Pr, Nd, Pm, Sm, Eu, Gd, Tb, Dy, Ho, Er, Tm, Yb and Lu. Note that lanthanum, La, may be classed as the first element in the lanthanide series, or alternatively as the first of the third row (sixth period) transition metal elements. For the purpose of the present invention, it is classed as the first element in the lanthanide series, i.e. as a lanthanide or rare earth element rather than a transition metal.

The first semiconductor may be present in a higher molar amount than the second semiconductor. Thus the molar amount of the first semiconductor in the particle is typically greater than the molar amount of the second semiconductor in the particle. For instance, the molar ratio of the first semiconductor to the second semiconductor may be from 1:1 to 500:1, or from 5:1 to 300:1, for instance from 25:1 to 250:1 or for example from 45:1 to 240:1.

The molar ratio of the first semiconductor to the second semiconductor may in some embodiments be from 25:1 to 75:1, for instance from 40:1 to 60:1, for example about 50:1.

In other embodiments, the molar ratio of the first semiconductor to the second semiconductor may be from 50:1 to 250:1, for instance from 75:1 to 200:1, for example from 80:1 and 150:1, or from 85:1 and 125:1, or for instance from 90:1 and 110:1.

In yet other embodiments, the molar ratio of the first semiconductor to the second semiconductor may be from 150:1 to 300:1, for instance from 200:1 to 250:1.

The first semiconductor may be present in a higher amount by mass than the second semiconductor. For instance, the mass ratio of the first semiconductor to the second semiconductor may be from 1:1 to 100:1, for instance from 2:1 to 75:1, or from 3:1 to 60:1, for instance from 5:1 to 50:1.

The mass ratio of the first semiconductor to the second semiconductor may in some embodiments be from 5:1 to 25:1, for instance from 5:1 to 20:1, for example from 5:1 to 15:1.

The mass ratio of the first semiconductor to the second semiconductor may in some embodiments be from 10:1 to 80:1, for instance from 20:1 to 70:1, for example from 30:1 to 60:1.

The mass ratio of the first semiconductor to the second semiconductor may in some embodiments be from 5:1 to 50:1, for instance from 10:1 to 30:1, for example from 15:1 to 25:1.

The molar ratio and mass ratio of the first semiconductor to the second semiconductor may be established using energy-dispersive X-ray spectroscopy (EDX). When a plurality of particles of the invention are present or are used as part of a therapy or treatment, the amounts above refer to the average (i.e. mean) ratio of the first semiconductor to the second semiconductor.

The compound of the first metal is typically an oxide of the first metal, where the first metal may be as further defined above. Typically, therefore, the first semiconductor comprises a metal oxide. The metal oxide is typically a transition metal oxide. The first material may comprise, consist essentially of or consist of the transition metal oxide.

Typically, the first semiconductor comprises titanium oxide (also referred to as titanium dioxide, titania, or TiO₂), zirconium oxide (ZrO₂), hafhium oxide (HfO₂), vanadium oxide, niobium oxide, tantalum oxide, tungsten oxide or molybdenum oxide. When the first semiconductor is niobium oxide, the first semiconductor is often Nb₂O₅. When the first semiconductor is tantalum oxide, the first semiconductor is often Ta₂O₅. Usually, the first semiconductor comprises titanium oxide.

The titanium dioxide may be in any amorphous or crystalline form. It may therefore be in, for example, anatase, rutile or brookite forms. Typically, the titanium dioxide is in the anatase form. Advantageously, the anatase form of titanium dioxide has a higher intrinsic photoactivity than the other forms of titanium dioxide.

The process of electron-hole recombination is suppressed by the use of titanium oxide as a core material due to the specific nature of the titanium oxide band structure and as a result single phase titanium oxide is photoactive.

In one embodiment, at least 80% by weight of the titanium dioxide is in the anatase form. It is preferred that at least 85% by weight, particularly at least 90% by weight, of the titanium dioxide is in the anatase form. Often, at least 95% by weight, especially at least 99% by weight, of the titanium dioxide is in the anatase form.

In some cases, the first semiconductor may comprise a transition metal oxide wherein the transition metal oxide is doped with a (at least one) dopant element which is a rare earth element, a transition metal or a p-block metal. For instance, the first semiconductor may comprise a transition metal oxide doped with a (at least one) dopant element selected from a lanthanide, tungsten (W), molybdenum (Mo), hafnium (Hf), indium (In), scandium (Sc) or gallium (Ga). The at least one dopant element is generally present as a dopant in the host lattice of the transition metal oxide, e.g. in the form of a cation.

When the first semiconductor comprises transition metal oxide, the transition metal oxide is typically not doped.

Thus, when the first semiconductor comprises titanium oxide, said titanium oxide is typically not doped.

Usually, the first semiconductor is not doped. Typically, therefore, the first semiconductor does not comprise a dopant element as defined above in the preceding paragraph.

Typically, the second semiconductor is not doped. Thus, often, the first semiconductor is not doped and the second semiconductor is not doped.

The compound of the second metal is typically an oxide of the second metal, where the second metal may be as further defined above. The second semiconductor typically therefore comprises a metal oxide. It may consist essentially of, or consist of, the metal oxide.

Often, for instance, the second semiconductor comprises a lanthanide oxide, yttrium oxide (Y₂O₃), hafhium oxide (HfO₂), zirconium oxide (ZrO₂), a tungstate compound or a tantalate compound. Typically, the second semiconductor comprises, consists essentially of or consists of a lanthanide oxide. The lanthanide oxide may be selected from lanthanum oxide, cerium oxide, praseodymium oxide, neodymium oxide, promethium oxide, samarium oxide, europium oxide, gadolinium oxide, terbium oxide, dysprosium oxide, holmium oxide, erbium oxide, thulium oxide, ytterbium oxide or lutetium oxide. All of the isotopes of promethium (Pm) are radioactive. It is therefore preferred that the lanthanide oxide is selected from lanthanum oxide, cerium oxide, praseodymium oxide, neodymium oxide, samarium oxide, europium oxide, gadolinium oxide, terbium oxide, dysprosium oxide, holmium oxide, erbium oxide, thulium oxide, ytterbium oxide or lutetium oxide. Typically the lanthanide oxide is of the form Ln₂O₃. For instance, the second semiconductor may be selected from La₂O₃, Ce₂O₃, Pr₂O₃, Nd₂O₃, Sm₂O₃, Eu₂O₃, Gd₂O₃, Tb₂O₃, Dy₂O₃, Ho₂O₃, Er₂O₃, Tm₂O₃, Yb₂O₃ or Lu₂O₃.

The second semiconductor may for instance be selected from lanthanum oxide, cerium oxide, praseodymium oxide, neodymium oxide, samarium oxide, europium oxide, terbium oxide, dysprosium oxide, holmium oxide, erbium oxide, thulium oxide, ytterbium oxide or lutetium oxide. For instance, the second semiconductor may be selected from La₂O₃, Ce₂O₃, Pr₂O₃, Nd₂O₃, Sm₂O₃, Eu₂O₃, Tb₂O₃, Dy₂O₃, Ho₂O₃, Er₂O₃, Tm₂O₃, Yb₂O₃ and Lu₂O₃.

Typically, the second semiconductor comprises ytterbium oxide (Yb₂O₃), lanthanum oxide (La₂O₃), gadolinium oxide (Gd₂O₃) or lutetium oxide (Lu₂O₃). Typically, the second semiconductor comprises lanthanum oxide (La₂O₃), gadolinium oxide (Gd₂O₃) or lutetium oxide (Lu₂O₃). The second semiconductor may for instance comprise lanthanum oxide (La₂O₃) or lutetium oxide (Lu₂O₃). The second semiconductor may for instance comprise lanthanum oxide (La₂O₃) or gadolinium oxide (Gd₂O₃). The second semiconductor may for instance comprise gadolinium oxide (Gd₂O₃) or lutetium oxide (Lu₂O₃). Typically, the second semiconductor comprises ytterbium oxide (Yb₂O₃), gadolinium oxide (Gd₂O₃) or lutetium oxide (Lu₂O₃).

In some embodiments, the first semiconductor comprises titanium dioxide and the second semiconductor comprises a compound selected from ytterbium oxide (Yb₂O₃), gadolinium oxide (Gd₂O₃) or lutetium oxide (Lu₂O₃). For instance, the first semiconductor may comprise titanium oxide and the second semiconductor may comprise ytterbium oxide. In some embodiments, the first semiconductor comprises titanium dioxide and the second semiconductor comprises a compound selected from lanthanum oxide (La₂O₃), gadolinium oxide (Gd₂O₃) or lutetium oxide (Lu₂O₃). For instance, the first semiconductor may comprise titanium oxide and the second semiconductor may comprise lanthanum oxide. The first semiconductor may comprise titanium oxide and the second semiconductor may comprises gadolinium oxide. The first semiconductor may comprise titanium oxide and the second semiconductor may comprise lutetium oxide.

The term “particle size”, as used herein, means the diameter of the particle if the particle is spherical or, if the particle is non-spherical, the volume-based particle size. The volume-based particle size is the diameter of the sphere that has the same volume as the non-spherical particle in question. The particle size takes into account the combined size of both the first and second semiconductors, and any coating if present.

Typically, a particle employed in the present invention has a size of less than 400 nm. This allows the particle to leave the blood stream of a human or animal body. It is preferred that the particle has a size less than 380 nm, especially less than 300 nm. Tumour vasculature is hyperpermeable and has pore sizes from 50 to 600 nm.

Large particles can be sequestered easily by the reticuloendothelial system and may be taken up by the liver or spleen or may be rapidly cleared from the body. It is preferred that a particle employed in the present invention has a size less than or equal to 100 nm. A particle having this size will avoid clearance by phagocytic uptake and hepatic filtration.

Small particles can easily pass through the leaky capillary wall of a tumour. However, the kidneys can also clear very small particles by glomerular filtration. It is preferred that a particle employed in the present invention has a size greater than or equal to 5 nm. A particle having this size will avoid clearance of the particles by the kidneys and to provide good particle retention in a tumour.

Typically, the particle employed in the present invention has a size of less than or equal to 400 nm, for instance less than or equal to 200 nm, less than or equal to 100 nm, or for instance from 1 to 100 nm. The particle size may for instance be from 5 to 75 nm, typically from 10 to 70 nm or from 10 to 65 nm, for instance from 20 to 70 nm, from 40 to 70 nm, or for example from 50 to 60 nm.

Such a size permits endocytosis of the particles into tumour cells. The particle size of the particle employed in the present invention may for example be from 5 to 95 nm, more typically from 5 to 85 nm (e.g. from 8 to 75 nm), and particularly from 10 nm to 70 nm. The size of the particle may be selected to allow it to enter a cell. For this purpose, the particle may have a size of equal to or less than 100 nm, but typically has a size of less than 100 nm, for instance a size of up to (i.e. equal to or less than) 70 nm. The particle may also be able to enter an organelle of a cell.

Normally, a distribution of particles having various sizes is obtained. Thus, when there are a plurality of particles of the invention, such as in a pharmaceutical composition, therapy or treatment of the invention, then the sizes described herein (e.g. in the preceding paragraphs) for a single particle refer to an average (i.e. the mean) size of the particles in a distribution. The average size of the particles in a distribution may be determined using standard centrifuge measurement techniques, dynamic light scattering, or analysis of electron micrograph images (for instance high resolution transmission electron microscopy images).

As described above, the particle typically comprises a core comprising one of the semiconductors. The core may be partially coated with the other of the two semiconductors. Accordingly, one semiconductor may be disposed on a surface of the other semiconductor such that portions of both the first and second semiconductors are exposed. In this situation, the size of the core may be from 1 to 100 nm, and is typically from 10 to 80 nm, for instance from 15 to 70 nm, or, for example, from 20 to 50 nm. The thickness of the coating may be from 1 to 50 nm, for instance from 1 to 40 nm, or from 1 to 30 nm, for example from 1 to 20 nm, and is usually from 1 and 15 nm, for instance from 1 to 10 nm, most preferably from 1 to 5 nm. It may for instance be from 2 to 4 nm, or for example from 2 to 3 nm.

In some embodiments, the particle comprises a core comprising the first semiconductor, the first semiconductor is titanium dioxide (typically undoped titanium dioxide), and the second semiconductor partially coats the core, the second semiconductor comprises lutetium oxide and the particle has a diameter of less or equal to than 100 nm. Typically, the particle has a diameter from 5 to 75 nm, typically from 10 to 65 nm, most typically from 50 to 60 nm. The particle may optionally further comprise a coating as described hereinbelow.

In other embodiments, the particle comprises a core comprising the first semiconductor, the first semiconductor comprises titanium dioxide (typically undoped titanium dioxide), and the second semiconductor partially coats the core, the second semiconductor comprises gadolinium oxide and the particle has a diameter of less than 100 nm. Typically, the particle has a diameter from 5 to 75 nm, typically from 10 to 65 nm, most typically from 50 to 60 nm. The particle may optionally further comprise a coating as described hereinbelow.

In some embodiments, the particle comprises a core comprising the first semiconductor, the first semiconductor comprises titanium dioxide (typically undoped titanium dioxide), and the second semiconductor partially coats the core, the second semiconductor comprises ytterbium oxide and the particle has a diameter of less than 100 nm. Typically, the particle has a diameter from 5 to 75 nm, typically from 10 to 65 nm, most typically from 50 to 60 nm. The particle may optionally further comprise a coating as described hereinbelow.

In some embodiments, the particle comprises a core comprising the first semiconductor, the first semiconductor comprises titanium dioxide (typically undoped titanium dioxide), and the second semiconductor partially coats the core, the second semiconductor comprises lanthanum oxide and the particle has a diameter of less than 100 nm. Typically, the particle has a diameter from 5 to 75 nm, typically from 10 to 65 nm, most typically from 50 to 60 nm. The particle may optionally further comprise a coating as described hereinbelow.

The particle of the invention, or each one of the particles in the composition of the invention, may optionally further comprise at least one further semiconductor, for instance a third semiconductor, or a third semiconductor and a fourth semiconductor. Typically each of the at least one further semiconductors, for instance the third semiconductor, or the third semiconductor and the fourth semiconductor, forms a heterojunction with the first semiconductor. The heterojunction is typically a staggered (Type II) heterojunction. Each of the at least one further semiconductors, for instance the third semiconductor, or the third semiconductor and the fourth semiconductor, typically comprises a metal which has a higher atomic number (Z) than the first metal (in the first semiconductor). Usually, each of the at least one further semiconductors, for instance the third semiconductor, or the third semiconductor and the fourth semiconductor, is a material as defined herein for the second semiconductor (although each further semiconductor will of course be different from the other semiconductors in the particle). Thus, each of the at least one further semiconductors, for instance the third semiconductor, or the third semiconductor and the fourth semiconductor, typically comprises: a lanthanide oxide, yttrium oxide, hafnium oxide, zirconium oxide, a tungstate compound or a tantalate compound (provided of course that it is different from the second semiconductor). Each of the at least one further semiconductors may for instance comprise a lanthanide oxide selected from lanthanum oxide, cerium oxide, praseodymium oxide, neodymium oxide, samarium oxide, europium oxide, gadolinium oxide, terbium oxide, dysprosium oxide, holmium oxide, erbium oxide, thulium oxide, ytterbium oxide or lutetium oxide, for instance lanthanum oxide, gadolinium oxide or lutetium oxide (provided of course that each of the at least one further semiconductors is different from the second semiconductor). For example, the second semiconductor may comprise lutetium oxide and a third semiconductor may comprise lanthanum oxide or gadolinium oxide. Alternatively, for example, the second semiconductor may comprise lutetium oxide, a third semiconductor may comprise gadolinium oxide, and a fourth semiconductor may comprise lanthanum oxide. Each of the at least one further semiconductors may for instance comprise a lanthanide oxide selected from ytterbium oxide, gadolinium oxide and lutetium oxide (provided of course that each of the at least one further semiconductors is different from the second semiconductor). For example, the second semiconductor may comprise lutetium oxide and a third semiconductor may comprise ytterbium oxide or gadolinium oxide. Alternatively, for example, the second semiconductor may comprise lutetium oxide, a third semiconductor may comprise gadolinium oxide, and a fourth semiconductor may comprise ytterbium oxide. Usually, each of the at least one further semiconductors is disposed on the surface of the first semiconductor. Each of the at least one further semiconductors may be disposed on the “first region” of the particle of the invention as defined hereinbefore.

The particle, or each particle in the plurality of particles as described herein, may further comprise a coating. The coating is typically a surface coating, i.e. a coating disposed on the outer surfaces of the semiconductors in the particle. The coating is typically disposed on the outer surfaces of the first and second semiconductors. In particular, the coating is typically disposed on the exposed surfaces of the first and second semiconductors (and indeed on the exposed surfaces of any further semiconductors that are present in the particle, e.g. a third semiconductor, or a third semiconductor and a fourth semiconductor). The coating may comprise (for instance, consist of) one or more of the following materials: silica (SiOₓ), alumina, and an organic coating, for instance polyethylene glycol, polystyrene, a saccharide, an oligosaccharide, a polyvinylpyrrolidone, a polyphosphate or a polysaccharide. The coating may comprise (for instance, consist of) a mixture of two, three or more of such materials. It should be noted that silica has a band gap of ˜9 eV, and is not therefore a semiconductor as defined herein. It should also be noted that alumina has a band gap of ˜7 eV, and is not therefore a semiconductor as defined herein. The coating may for instance be an organic coating, such as PEG, that enhances steric stabilisation. The inclusion of a coating on the particles can improve their biocompatibility, prevent them from agglomerating in vivo and allow them to be functionalised with other agents, for instance with one or more targeting moieties as described above. For instance, the particle according to the present invention may further comprise a negatively charged surface coating. The charge of the surface coating may be ascertained by measuring the zeta potential of the particles. Negatively charged surface coatings have the advantage that they can improve cellular uptake of the particles (see Patil et al., Protein adsorption and cellular uptake of cerium oxide nanoparticles as a function of zeta potential, Biomaterials 28, 2007, 4600-4607). Examples of negatively charged surface coating include polyphosphates, for example hexametaphosphate, or silica (SiOₓ). Typically, the coating comprises, or is, hexametaphosphate.

Any reference to the particle size of a particle of the invention refers to the total size of the particle, including any coating that may be present. When there is a plurality of particles such that the size is an average particle size, then the size refers to the average total size, including any coating(s) that may be present, of the particles. In general, the thickness of the coating is from 0.1 to 10 nm, typically from 1 to 5 nm. It is preferred that the coating is silica or an organic coating (for instance PEG, sucrose or a polyphosphate such as hexametaphosphate). Typically, the coating is silica. More typically, the particle comprises a silica coating with a thickness of less than 5 nm.

The thin (<5 nm) silica surface coating layer acts to make the device biocompatible and to induce surface charge to aid dispersion of the colloid. It must be thin in order to allow charge to undergo quantum tunneling through the silica barrier and interact with water on the surface. The thickness of the coating may be measured using high resolution transmission electron microscopy.

A targeting moiety may be attached or conjugated to the particle, or to each one of the particles, for instance to the surface of the or each particle, or to a coating on the surface of the or each particle. This may be achieved by attaching or conjugating to the particles a targeting moiety that possesses a high affinity for a molecular signature or structure found predominantly or exclusively in the malignant cells. The targeting moiety has a preferential binding affinity for a biological moiety, such as a molecular signature or structure (e.g. a gene, a protein, an organelle, such as mitochondria), which is generally only present in a cancer cell or a tumour tissue. The targeting moiety is capable of concentrating the particles in the tumour tissue or cancer cells. A particle as defined herein may therefore comprise at least one targeting moiety. A targeting moiety may be attached to a coating of a particle, for instance a silica coating disposed on the surface of the particle, as described in International patent application no. PCT/GB2010/002247 (WO 2011/070324). Alternatively, a targeting moiety may be attached to a coating of a particle wherein the coating comprises a polyphosphate, for instance wherein the coating comprises a hexametaphosphate. The targeting moiety may be a peptide, a polypeptide, a nucleic acid, a nucleotide, a lipid, a metabolite, an antibody, a receptor ligand, a ligand receptor, a hormone, a sugar, an enzyme, a vitamin or the like. For example, the targeting moiety may be selected from a drug (e.g. trastuzumab, gefitinib, PSMA, tamoxifen/toremifen, imatinib, gemtuzumab, rituximab, alemtuzumab, cetximab), a DNA topoisomerase inhibitor, an antimetabolite, a disease cell cycle targeting compound, a gene expression marker, an angiogenesis targeting ligand, a tumour marker, a folate receptor targeting ligand, an apoptotic cell targeting ligand, a hypoxia targeting ligand, a DNA intercalator, a disease receptor targeting ligand, a receptor marker, a peptide (e.g. a signal peptide, a melanocyte stimulating hormone (MSH) peptide), a nucleotide, an antibody (e.g. an antihuman epidermal growth factor receptor 2 (HER2) antibody, a monoclonal antibody C225, a monoclonal antibody CD31, a monoclonal antibody CD40), an antisense molecule, an siRNA, a glutamate pentapeptide, an agent that mimics glucose, amifostine, angiostatin, capecitabine, deoxycytidine, fullerene, herceptin, human serum albumin, lactose, quinazoline, thalidomide, transferrin and trimethyl lysine. Typically, the targeting moiety is a nuclear localization signal (NLS) peptide.

Accordingly, the particle employed in the present invention, or each particle in the plurality of particles employed in the invention, may further comprise a targeting moiety. The targeting moiety may be attached or conjugated to the or each particle, for instance to the surface of the or each particle, or to a coating on the surface of the or each particle. The particle of the invention may therefore further comprise a coating—typically a silica coating or a hexametaphosphate coating—as defined herein and a targeting moiety.

An optical contrast agent, a radioisotope, a paramagnetic contrast agent or a superparamagnetic contrast agent may also be attached to the coating, either with or without a targeting moiety as described above. The contrast agent may be a gadolinium MRI contrast agent.

Accordingly, the particle, or each one of the particles, may comprise, consist essentially of, or consist of:

(i) the first semiconductor, which may be as defined anywhere herein, which is optionally doped with at least one dopant element as defined herein but is typically not doped;

(ii) the second semiconductor, which may be as defined anywhere herein;

optionally, (iii) at least one further semiconductor, for instance a third semiconductor, or a third semiconductor and a fourth semiconductor, which may be as defined hereinbefore;

optionally, (iv) a coating which may be as defined herein;

optionally, (v) a targeting moiety as defined herein; and

optionally, (vi) an optical contrast agent, a radioisotope, a paramagnetic contrast agent or a superparamagnetic contrast agent.
Generally, the electrons in the particle are capable of being excited by X-rays, gamma rays, protons, electrons (beta rays), positrons or alpha particles. When an electron is excited by incoming radiation, it moves to the conduction band, leaving a hole in the valence band. In order to optimise water splitting the particle must split the electron and hole into separate regions in order to minimise radiative combination and maximise the potential for de-excitation via water splitting. This can be mediated either via the electron, as;

e⁻+O₂→^{•}O₂⁻

Or the hole as;

h⁺+H₂O→H⁺+OH^{•}

In order for the splitting of water to proceed, it must be energetically favourable in that energy is lost by transitions of electrons from the conduction band to the oxygen level and by holes to the water level. Therefore, typically the particle is capable of generating hydroxyl free radicals from water when subjected to ionising radiation in the presence of water. Additionally, the particle may be capable of generating superoxide free radicals from oxygen when subjected to ionising radiation in the presence of oxygen.

Once formed superoxide and hydroxyl free radicals may be used to damage cellular components. Holes generate free radicals by water splitting and may therefore be used irrespective of the oxygen level of the tumour regions, i.e. hypoxic regions may be targeted. These regions have traditionally been harder to target. Hydroxyl radicals are believed to oxidize the membrane lipids of cells to produce peroxidants, which then set up a series of peroxidant chain reactions; the oxidatively stressed malignant cells progress to a necrotic state that results in their destruction. Typically, the particle is suitable for use in combination with ionising radiation for the destruction of cellular components.

Owing to the ability of the particles of the present invention to generate free radicals when subjected to ionising radiation, the invention also relates to a process for producing free radicals, comprising exposing a particle of the invention to ionising radiation in the presence of water. The process may be performed in vivo, e.g. in a medical treatment as described herein. Often, however, the process is not performed in vivo. Thus, it is often an ex vivo process. It may for instance be for water purification. Thus, the process of the invention may be an ex vivo process for the purification of water, comprising exposing a particle of the invention to ionising radiation in the presence of water to be purified. The ROS generated act as a biocide, for instance to kill bacteria in the water.

The process typically comprises generating hydroxyl free radicals from the water.

Optionally, the process comprises exposing a particle of the invention to ionising radiation in the presence of oxygen and water. Thus, additionally, the process may comprise generating superoxide free radicals from the oxygen.

Typically, the ionising radiation comprises at least one selected from X-rays, gamma rays, protons, electrons (beta rays), positrons and alpha particles.

Particles of the invention, which comprise different semiconductors that are in contact with one another such that a heterojunction is formed therebetween, may be produced by crystallisation or precipitation from a solution which comprises soluble precursors to each of the semiconductors. The solution usually contains a first precursor compound and a second precursor compound and a solvent. The first and second precursor compounds are soluble precursors to the first and second semiconductors respectively. Any suitable solvent or mixture of solvents, which is capable of dissolving the precursor compounds in question, is employed. Typically, a mixture of organic solvents and water is chosen. As discussed above, the first semiconductor is usually a compound (for example an oxide) of a first metal and the second semiconductor is usually a compound (for example an oxide) of a second metal. The first and second precursor compounds are usually therefore soluble salts of the first and second metals respectively, i.e. salts of the first and second metals which are soluble in the chosen solvent. Metal nitrates, halides, sulphides, sulphates, acetates, oxysulphides and alkoxides may for instance be employed. When the first semiconductor is titanium oxide, titanium(IV) (triethanolaminato) isopropoxide solution is typically employed, and when the second semiconductor is a lanthanide oxide, a nitrate of the lanthanide is often used. Often, the solvent employed comprises isooctane, butanol and deionized water. Other agents may also be present, for example surfactants, salts and/or buffers to aid dissolution of the starting materials, control the ionic strength of the solution and aid crystallisation or precipitation of the particles. Typically, the surfactant dioctyl sulfosuccinate sodium salt, NaCl and NaOH are employed. The first semiconductor is typically formed in an amorphous phase prior to addition of the second semiconductor amorphous phase. The composite particles are then typically crystallised from the solution in a hydrothermal reactor at an elevated temperature, usually at temperature of from 150° C. to 200° C., for example 170° C. The solution is usually held at that temperature for at least an hour, to enable crystallisation. The solid, crystallised product is then isolated by centrifugation and washed in a suitable solvent, e.g. an alcohol such as, for instance, isopropanol. The isolated product is typically then further crystallised by firing at a high temperature (e.g. at least 500° C., for example about 700° C.) for a relatively short period of time, for instance for at least ten minutes. About fifteen minutes is typical. The product is then allowed to cool to yield the particles of the invention.

Particles of the invention, which comprise different semiconductors that are in contact with one another such that a heterojunction is formed therebetween, may be also produced by depositing a second semiconductor on particles of the first semiconductor. As discussed above, the first semiconductor is usually a compound (for example an oxide) of a first metal and the second semiconductor is usually a compound (for example an oxide) of a second metal. In this process, a dispersion of particles of the first semiconductor is dispersed in a solvent. Typically, the solvent is water. A solution of a second precursor compound, as described above, is added to the dispersion. Typically, the second precursor compound is a metal nitrate, wherein the metal is any metal as described herein in relation to the second semiconductor. Optionally, the pH may be adjusted to a desired value by adding an acid or alkali. Typically, the pH is adjusted to a value of at least 5, for instance from 5 to 14, or from 5 to 12 or from 5 to 10, preferably from 6 to 8. Typically the pH is adjusted to between 6 and 8 by adding an alkaline solution, for example an alkali metal hydroxide solution such as potassium hydroxide. The particles are then isolated from the mixture by any method known to the skilled person, for instance by filtration or centrifugation. Typically the particles are then dried by any method known to the skilled person, for instance by freeze drying. Typically the particles are isolated by centrifugation, then freeze dried. Typically, the isolated particles are then heat treated. Thus, the particles are typically heated. Usually, the particles are heated for up to an hour, more typically for up to half an hour, for instance for up to 10 minutes. Often, they are heated for from 5 to 10 minutes. For instance, the particles may be heated to a temperature of at least 100° C., at least 200° C., at least 300° C., at least 400° C. or at least 500° C. Typically, the particles are heated to a temperature of between 500 and 1000° C., for instance between 600 and 900° C., between 700 and 800° C., for instance about 750° C. Usually, the particles are heated to the temperature for up to an hour, more typically for up to half an hour, for instance for up to 10 minutes. Often, they are heated for from 1 minute up to any of the aforementioned durations, for instance for from 1 minute to 10 minutes, for example from 5 minutes to 10 minutes. Often, they are heated for about 8 minutes.

A silica surface coating layer may optionally then be added to the particles by treating a suspension of the particles with a silica precursor compound, for instance tetraethyl orthosilicate (TEOS) and then stirring for an hour or so, followed by washing in a suitable solvent (e.g. an alcohol such as, for instance, isopropanol) prior to dispersion in water and freeze drying.

A polyphosphate coating may optionally be added to the particles by adding a polyphosphate salt, typically sodium hexametaphosphate, to a dispersion of the particle or particles. Typically, the weight ratio of particles to polyphosphate salt added is at least 1:1, for instance from 1:1 to 5:1 particles:polyphosphate salt. Typically, the weight ratio of particles to polyphosphate salt added is 2:1.

Particles of the invention may be formulated into pharmaceutical compositions of the invention. The invention provides a pharmaceutical composition which comprises (i) a plurality of particles comprising a first semiconductor and a second semiconductor wherein the first semiconductor forms a heterojunction with the second semiconductor, and optionally (ii) one or more pharmaceutically acceptable ingredients.

In some aspects, the pharmaceutical composition may be used in combination with radiotherapy comprising irradiating a site of the cancer with radiation from an external source or with radiotherapy using a radioactive material inside the subject (internal radiotherapy), as further defined herein below for the treatment of cancer in a subject. Therefore, the pharmaceutical composition as defined above may also comprise a radioactive material (such as a radiopharmaceutical or radioactive embolization particles) suitable for internal radiotherapy.

The invention also therefore provides a pharmaceutical composition as defined above which comprises said plurality of particles of the invention and further comprises a radioactive material (such as for instance a radiopharmaceutical or radioactive embolization particles) suitable for internal radiation therapy.

Any of the pharmaceutical compositions suitable for use in the treatments of the invention may further comprise a chemotherapeutic agent or an immunotherapeutic agent. The chemotherapeutic agent or immunotherapeutic agent may be as further defined hereinbelow.

Such pharmaceutical compositions, as described herein, typically further comprise one or more pharmaceutically acceptable ingredients. Suitable pharmaceutically acceptable ingredients are well known to those skilled in the art and include pharmaceutically acceptable carriers (e.g. a saline solution, an isotonic solution), diluents, excipients, adjuvants, fillers, buffers, preservatives, anti-oxidants, lubricants, stabilisers, solubilisers, surfactants (e.g. wetting agents), masking agents, colouring agents, flavouring agents and sweetening agents. Suitable carriers, diluents, excipients, etc. can be found in standard pharmaceutical texts. See, for example, Handbook for Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

A pharmaceutical composition may be in the form of (i.e. be formulated as) a liquid, a solution or a suspension (e.g. an aqueous or a non-aqueous solution), an emulsion (e.g. oil-in-water, water-in-oil), an elixir, a syrup, an electuary, a tablet (e.g. coated tablets), granules, a powder, a lozenge, a pastille, a capsule (e.g. hard and soft gelatine capsules), a pill, an ampoule, a bolus, a tincture, a gel, a paste or an oil.

Typically, the particles employed in the invention are dissolved in, suspended in, or admixed with one or more pharmaceutically acceptable ingredients.

A pharmaceutical composition comprising the particles suitable for topical administration may be in the form of a gel, cream, spray or paint. Following tumour resection local reoccurrence is common and can be devastating since further surgery is often not indicated. Local reoccurrence is caused by small regions of unresected tumour remaining following surgery. A pharmaceutical composition in the form of a gel, cream, spray or paint can be used on the tumour bed following resection, prior to radiotherapy on the tumour bed. The composition will enhance the effectiveness of radiotherapy treatment of tumour beds and reduce local reoccurrence of the tumour. In this case, the particles may be labelled with active targeting to further enhance the uptake into tumour cells—the topical administration of the composition means that long circulation times in the blood supply are not required and active targeting is feasible.

Accordingly, in one embodiment the pharmaceutical composition as defined above is suitable for topical administration. For instance, the pharmaceutical composition may be a gel, cream, spray or paint which comprises said plurality of particles. Such a composition may be applied directly to a site of a cancer prior to radiotherapy. Topical administration is particularly suitable when the site of the cancer is a region of unresected tumour following surgery. In this case, the cancer is often, for example, a cancer of the bowel, colon, rectum or brain. The pharmaceutical composition suitable for topical administration may comprise further ingredients such as water, alcohols, polyols, glycerol, vegetable oils, and the like; anti-oxidants, buffers, preservatives, stabilisers, bacteriostats, suspending agents, thickening agents, and solutes.

A pharmaceutical composition suitable for parenteral administration (e.g. by injection, for instance by intratumoral injection) may include an aqueous or non-aqueous, sterile liquid in which the particles employed in the invention are suspended or dispersed. Such liquids may additionally contain other pharmaceutically acceptable ingredients, such as anti-oxidants, buffers, preservatives, stabilisers, bacteriostats, suspending agents, thickening agents, and solutes that render the formulation isotonic with the blood (or other relevant bodily fluid) of the intended recipient. Examples of excipients include water, alcohols, polyols, glycerol, vegetable oils, and the like. Examples of suitable isotonic solutions for use in such formulations include Sodium Chloride Injection, Ringer's Solution or Lactated Ringer's Injection. Phosphate buffered saline may for instance be employed (as described in Example 2 herein) as an aqueous liquid in which the particles employed in the invention are suspended.

Therefore, typically, the pharmaceutical composition is suitable for administration by injection, for instance intratumoral injection. Often, this pharmaceutical composition comprises a plurality of particles as described herein dispersed in an aqueous solution. The concentration of the particles in the aqueous solution is typically from 0.1 mg·ml⁻¹ to 500 mg·ml⁻¹. Usually, for instance, the concentration of the particles in the aqueous solution is from 0.5 mg·ml⁻¹ to 200 mg·ml⁻¹, for example from 1.0 mg·ml⁻¹ to 100 mg·ml⁻¹. The aqueous solution may for instance comprise from 3 mg·ml⁻¹ to 80 mg·ml⁻¹, or for instance from 5 mg·ml⁻¹ to 60 mg·ml⁻¹, of the particles. The aqueous solution is preferably a glucose solution. For example, the aqueous solution may comprise at least 1% by weight glucose, for instance from 1 to 20% by weight glucose, from 1 to 10% by weight glucose, from 2 to 8% by weight glucose or about 5% by weight glucose. The aqueous solution may further comprise a polyphosphate salt, for example a hexametaphosphate salt, for instance sodium hexametaphosphate. Typically, the weight ratio of particles to polyphosphate salt in the solution is at least 1:1, for instance from 1:1 to 5:1 particles:polyphosphate salt. Typically, the weight ratio of particles to polyphosphate salt added is 2:1. The polyphosphate may form a coating as described herein on the particles.

The pharmaceutical composition may be presented in unit-dose or multi-dose sealed containers. Extemporaneous injection solutions and suspensions may be prepared from sterile powders, granules, and tablets.

A pharmaceutical composition suitable for oral administration (e.g. by ingestion) includes a liquid, a solution or suspension (e.g. aqueous or non-aqueous), an emulsion (e.g. oil-in-water, water-in-oil), an elixir, a syrup, an electuary, a tablet, granules, a powder, a capsule, a pill, an ampoule or a bolus.

Tablets may be made by conventional means e.g. by compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); preservatives (e.g. methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated, for example, to affect release (e.g. an enteric coating to provide release in parts of the gut other than the stomach).

In general, the pharmaceutical composition will comprise a therapeutically effective amount of the particles of the invention. The term “therapeutically effective amount” as used herein, refers to the amount of the particles of the invention, whether as part of a pharmaceutical composition, kit or otherwise, which is effective for producing some desired therapeutic effect when administered in accordance with a desired treatment regimen and when the subject is treated with a prescribed dosage of radiotherapy.

It will be appreciated by one of skill in the art that appropriate dosages of the particles and a pharmaceutical composition comprising the particles can vary from patient to patient. Determining the optimal dosage will generally involve balancing of the level of therapeutic benefit against any risk or deleterious side effects. The selected dosage level will depend on a variety of factors including the route of administration, the time of administration, the rate of excretion of the particles, the duration of the treatment, other compounds and/or materials used in combination, the severity of the condition, and the species, sex, age, weight, condition, general health, and prior medical history of the patient. The amount of particles and route of administration will ultimately be at the discretion of the physician, veterinarian, or clinician, although generally the dosage will be selected to achieve local concentrations at the site of action that achieve the desired effect.

The concentration of the particles in a pharmaceutical composition of the invention (for instance in a pharmaceutical composition suitable for parenteral administration, as defined above) is typically from 0.1 mg·ml⁻¹ to 500 mg·ml⁻¹. Usually, for instance, the concentration of the particles in the pharmaceutical composition is from 0.5 mg·ml⁻¹ to 200 mg·ml⁻¹, for example from 1.0 mg·ml⁻¹ to 100 mg·ml⁻¹. The pharmaceutical composition may for instance comprise from 3 mg·ml⁻¹ to 80 mg·ml⁻¹, or for instance from 5 mg·ml⁻¹ to 60 mg·ml⁻¹, of the particles.

The number concentration of the particles in the pharmaceutical composition may, for instance, be from 1×10¹⁰ particles/ml to 1×10²⁴ particles/ml, for example from 1×10¹³ particles/ml to 1×10²¹ particles/ml, such as for instance from 1×10¹⁵ particles/ml to 1×10¹⁸ particles/ml.

When the pharmaceutical composition comprising the plurality of nanoparticles further comprises a radioactive material suitable for internal radiation therapy, the concentration of the radioactive material in the composition will depend on the particular radioactive material being employed and the target dose of radiation, and may be calculated by the clinician by using methods known in the art for particular known radioactive materials employed in particular known internal radiation therapy procedures.

For instance, when radioactive embolization particles are employed for internal radiation therapy, e.g. radioactive embolization particles as further described herein such as beta emitting yttrium-90 SIRT beads, the concentration of the embolization particles in the pharmaceutical composition may, for example, be from 0.05 mg·ml⁻¹ to 50 mg·ml⁻¹, or for instance from 0.1 mg·ml⁻¹ to 20 mg·ml⁻¹, for example from 0.2 mg·ml⁻¹ to 5 mg·ml⁻¹.

If a radiopharmaceutical is being employed for internal radiation therapy, again the concentration of the radiopharmaceutical in the pharmaceutical composition will of course depend on the particular radiopharmaceutical and the target dose of radiation. The concentration may for instance be selected such that a dose of from 10 to 100 kBq per kg of body weight is achieved in a single injection, for instance a dose of from 35 to 65 kBq per kg of body weight.

The present invention also relates to a particle of the invention as defined herein, or a pharmaceutical composition of the invention as defined herein, for use in the treatment of the human or animal body by therapy.

The present invention also relates to a particle of the invention as defined herein, or a pharmaceutical composition of the invention as defined herein, for use in combination with radiotherapy in the treatment of cancer in a subject.

The invention also relates to methods and uses for treating, or for the treatment of, cancer, in combination with radiotherapy.

The term “treatment” as used herein in the context of treating cancer refers generally to treatment and therapy, whether of a human or an animal (e.g. in veterinary applications), in which some desired therapeutic effect is achieved, such as, for example, the inhibition of the progress of the condition. The term includes a reduction in the rate of progress, a halt in the rate of progress, regression of the condition, amelioration of the condition, and cure of the condition. Palliative treatment or treatment as a prophylactic measure (i.e. prophylaxis, prevention) are also included.

The subject may be a human or a non-human. The subject is typically a mammal, for instance a human, or a non-human mammal. Usually, the subject is a human. The subject may be referred to herein as a patient. The subject may for instance be a human patient.

Radiotherapy, i.e. radiation therapy, uses high-energy radiation to shrink tumours and kill cancer cells. X-rays, gamma rays and charged particles (e.g. electrons, protons, positrons, alpha particles) are examples of types of radiation used for cancer treatment. The radiation may be delivered by a machine outside the body (external radiotherapy), or it may come from radioactive material placed in the body, within or near cancer cells (internal radiotherapy). The term “internal radiation therapy”, as used herein, therefore means radiotherapy (i.e. radiation therapy) in which the radiation is delivered from a radiation source (a radioactive material) located inside the subject's body. The radiation source (a radioactive material) is generally located at or near to a site of the cancer to be treated, for example within or near to a cancerous tumour. The internal radiation therapy may be brachytherapy. Alternatively, internal radiation therapy may be performed using a radiopharmaceutical, i.e. a radioactive drug, which is typically swallowed or administered parenterally. Another method is to use radioactive embolization particles.

Therefore, a particle or pharmaceutical composition of the present invention may be used in combination with: (i) radiotherapy which comprises irradiating a site of the cancer with radiation from an external source, or (ii) radiotherapy using a radioactive material inside the subject.

Thus, the radiotherapy employed in the present invention may comprise irradiating a site of the cancer with radiation from an external source or from a radioactive material inside the subject.

Typically, the radiotherapy uses a source of energy of more than 50 keV, for instance a source of energy equal to or greater than 60 keV. It is typically greater than 60 keV, for instance it may be equal to or greater than 70 keV, for instance equal to or greater than 80 keV, or equal to or greater than 100 keV. The radiotherapy may for instance use a source of energy equal to or greater than 200 keV, for instance equal to or greater than 400 keV.

The radiotherapy may for instance comprise supplying X-ray or gamma ray photons with an incident energy of more than 50 keV, for instance with an incident energy equal to or greater than 60 keV, for example equal to or greater than 70 keV, or equal to or greater than 80 keV. The radiotherapy may comprise supplying X-ray or gamma ray photons with an incident energy equal to or greater than 100 keV, for instance with an incident energy equal to or greater than 200 keV, or equal to or greater than 400 keV. The photons may for instance have an incident energy of from 0.05 MeV (50 keV) to 10 MeV, for instance from 0.06 MeV (60 keV) to 10 MeV, or for instance from 0.08 MeV (80 keV) to 10 MeV, for example from 0.1 MeV (100 keV) to 1 MeV. The photons may for instance have an incident energy of from 0.2 MeV (200 keV) to 10 MeV, for instance from 0.4 MeV (400 keV) to 10 MeV.

Alternatively, the radiotherapy may comprise supplying electrons, positrons or protons with an incident energy that is equal to or greater than 10 MeV, for instance with an incident energy that is equal to or greater than 50 MeV. The incident energy may for instance be from 60 MeV to 300 MeV, for example from 70 MeV to 250 MeV. The treatment may for instance use, proton beam radiation (proton beam therapy) wherein the incident energy is thus defined, for example is from 70 MeV to 250 MeV.

When the radiotherapy comprises irradiating a site of the cancer with radiation from an external source, the radiotherapy may employ X-rays, gamma rays, electrons or protons. For instance, the radiotherapy may be selected from conformal radiotherapy, intensity modulated radiotherapy (IMRT), image guided radiotherapy (IGRT), 4-dimensional radiotherapy (4D-RT), stereotactic radiotherapy and radiosurgery, proton therapy, electron beam radiotherapy, and adaptive radiotherapy.

Conventionally, external radiotherapy uses a single external beam, usually of X-rays, with the patient being exposed from several directions such as front and back or side to side. Although the technology is very well established it is limited in its ability to spare normal tissue from excessive dose. Recent developments have included stereotactic radiosurgery (SRS) in which highly focused beams are used to target well defined tumour regions typically in the brain or spine. It is claimed that the ability to accurately target tumour regions and use shorter treatment regimes enhances the treatment efficacy. A typical example of a SRS system is Cyberknife™, which has had FDA clearance for treatment of tumours in any part of the body since 2001. The radiotherapy source is mounted on a robot arm and can deliver a pencil thin beam of radiation at 6-8Gy per minute. Again the main rationale for this approach is to increase the dose accuracy to the tumour and deliver dose escalation. Intensity modulated radiation therapy (IMRT) utilises multiple radiation beams to deliver maximum energy into fields that accurately map even complex tumour structures such as those wrapping around blood vessels. Medical professionals are required to map the structure one image at a time prior to devising a treatment protocol. There is increasing evidence of advanced survival using both SRS and IMRT techniques and reduced toxicity and normal tissue damage.

Proton therapy uses an external beam of protons to target the tumour site, the advantage being an ability to target a tumour mass more easily than using X-ray radiotherapy. This is due to the protons having limited side scatter due to their high mass and a well-defined penetration depth. In a similar fashion to X-ray based treatments, the protons my either directly damage DNA by scattering or indirectly by free radical generation.

Often, when the radiotherapy comprises irradiating a site of the cancer with radiation from an external source, X-ray radiation is used.

After administering the particles to a subject, a period of time sufficient to allow the particles to accumulate at the site of the cancer or tumour is usually allowed to elapse before directing X-ray radiation to the cancer. The time period between administration of the particles and irradiation with X-rays will depend on, amongst other things, the mode of administration, whether there is a targeting moiety attached to the particles and the nature of the cancer.

The step of directing X-ray radiation to a site of the cancer or tumour tissue may be carried out at least 3 hours, especially at least 6 hours, typically 9 to 48 hours, particularly 12 to 24 hours, after administering, typically orally or parenterally (including but not limited to intratumoural injection), the particle or the pharmaceutical composition to the subject.

When the pharmaceutical composition comprising the plurality of particles is used in combination with radiotherapy comprising irradiating a site of the cancer with radiation from an external source, the dose of radiation will depend on the type of radiation and the area of the body on which it is being deployed. Typically, the maximum dose that can be applied is 70-74Gy. In radiosensitive organs this may be reduced. The external radiotherapy can be administered in one dose, continuously or intermittently (e.g. in divided doses at appropriate intervals) throughout the course of the treatment. Single or multiple doses can be carried out with the dose level and pattern being selected by the treating physician, veterinarian, or clinician.

Generally, the subject is exposed to a total X-ray dose of from 20 to 70 Gy, such as for example 40 to 50 Gy.

Typically, a treatment or method for treating cancer of the invention comprises directing a 1.0 to 3.0 Gy, typically 1.5 to 2.5 Gy dose, more typically a 1.8 to 2.0 Gy dose of X-ray radiation to a site of the cancer or tumour tissue. Such small frequent doses are intended to allow healthy cells time to grow to repair any damage caused by the radiation.

Typically, the X-ray radiation has an incident energy of more than 50 keV, for instance an incident energy equal to or greater than 60 keV, for example equal to or greater than 70 keV, or equal to or greater than 80 keV. The X-ray radiation may for instance have an incident energy that is equal to or greater than 100 keV, for instance an incident energy equal to or greater than 200 keV, or equal to or greater than 400 keV. The X-ray radiation may for instance have an incident energy of from 0.05 MeV (50 keV) to 10 MeV, for instance from 0.06 MeV (60 keV) to 10 MeV, or for instance from 0.08 MeV (80 keV) to 10 MeV, for example from 0.1 MeV (100 keV) to 1 MeV. The X-ray radiation may for instance have an incident energy of from 0.2 MeV (200 keV) to 10 MeV, for instance from 0.4 MeV (400 keV) to 10 MeV.

The treatment may also comprise a step of detecting the presence or absence of a particle or particles of the invention at a locus or site of the cancer or tumour tissue before directing X-ray radiation to a locus or site of the cancer or tumour tissue. The detecting step may be performed as described below.

When the radiotherapy comprises irradiating a site of the cancer with radiation from a radioactive material inside the subject, the radiotherapy may be brachytherapy, for instance, or the radioactive material inside the subject may comprise a radiopharmaceutical or, for instance, radioactive embolization particles. The radioactive material may comprise a radioisotope which emits γ-radiation or a radioisotope which emits electrons through β-decay or a radioisotope which emits α-particles, or a radioisotope which emits a combination of these.

The internal radiotherapy may comprise brachytherapy. Brachytherapy generally involves placing a small piece or pieces of radioactive material inside the body, either temporarily or permanently, near the cancerous cells. Typically, the radioactive material comprises radioactive sources. Such radioactive sources are typically implanted into the site of the cancer, for instance into a tumour. The radioactive sources may be in the form of needles, tubes, wire, pellets or seeds and are generally used as sealed sources, placed inside shielding, to protect from radioactive leakage inside the body and/or the emission of types of radiation which are unwanted. Radioactive sources which are typically employed in brachytherapy are given in Table 1 below. Table 1 also gives the type of emission, half-life and energy for each source. Accordingly, the radioactive material employed when the internal radiotherapy employed in the present invention comprises brachytherapy, may, for instance, comprise any one of the radionuclides listed in table 1, or the radioactive material may comprise a mixture of any two, three, four, five or all six of those radionuclides in Table 1.

TABLE 1
Radioactive sources typically employed in brachytherapy

| Radionuclide | Emission type | Half life | | Energy | |
|---|---|---|---|---|---|
| 192-Ir | γ | 73.8 | days | 0.38 | MeV (mean) |
| 137-Cs | β⁻ | 30.17 | years | 0.662 | MeV |
| 60-Co | β⁻ | 5.25 | years | 1.17, 1.33 | MeV |
| 131-Cs | Electron capture | 9.7 | days | 30.4 | keV |
| 125-I | Electron capture | 59.6 | days | 27.4, 31.4, 35.5 keV | |
| 103-Pd | Electron capture | 17 | days | 21 | keV (mean) |

The three emission types employed are γ radiation, β⁻ radiation and electron capture. Discussing these in turn, γ radiation consists of high energy photons emitted from a nucleus during de-excitation from a high to low energy state. Once emitted into the tumour γ radiation high energy photons interact in the same way as external beam radiotherapy-generating electrons by Compton scattering off outer shell electrons within soft tissue, bone, etc. These photogenerated electrons then de-excite by generating a cascade of lower energy electrons until finally an electron interacts with molecular oxygen to create a superoxide free radical. This then damages cellular components.
β⁻ radiation consists of electrons emitted from a nucleus during the decay of a neutron to a proton. β⁻ electrons generate cascades of lower energy electrons until, they too, interact with molecular oxygen to create superoxide free radicals and damage cellular components.

Electron capture consists of high energy photons emitted from a nucleus following inner shell electron capture by a nuclear proton and consequent generation of a nuclear neutron. Photons are emitted following de-excitation from the excited state generated by the electron capture. The high energy photons then generate electrons by scattering off outer shell electrons within soft tissue or bone, and the photogenerated electrons then de-excite by generating a cascade of lower energy electrons until finally an electron interacts with molecular oxygen to create a superoxide free radical. This then damages cellular components.

In consequence of the reliance of γ radiation, β⁻ radiation and electron capture in brachytherapy on molecular oxygen, hypoxic tumour regions could not previously be targeted and treated effectively, a limitation which the present invention overcomes by employing particles as defined herein, which comprise a first semiconductor and a second semiconductor wherein the first semiconductor forms a heterojunction with the second semiconductor. These are used to convert energetic incident electrons into hydroxyl free radicals by a valance band hole mediated water splitting reaction.

h⁺+H₂O→H⁺+OH^{•}

The hole thus generated migrates to the top of the valance band. Owing to the presence of the two semiconductors, the likelihood of electron-hole recombination is minimised. The energy is the converted to hydroxyl free radicals by recombination of the photogenerated hole with external electrons by splitting of water. The hydroxyl free radical generated may damage cellular components via a normal electron exchange interaction.

Brachytherapy is particularly applicable to the treatment of solid tumours including sarcoma and tumours in the prostate, cervix, breast, lung, head and neck and oesophagus. Typically the cancer which is treated in accordance with the invention when the internal radiation therapy is brachytherapy, is prostate cancer, cancer of the oral cavity, throat cancer, oropharyngeal cancer, sarcoma, lung cancer, cervical cancer, oesophageal cancer or breast cancer. Typically, therefore, when the internal radiation therapy is brachytherapy, the site of the cancer comprises a tumour in the prostate, head, neck, oral cavity, throat, oropharynx, connective tissue, non-epithelial tissue, lung, cervix, oesophagus or breast. Typically, the tumour comprises a hypoxic region, as defined hereinbefore. Usually, the radioactive material is typically located within the tumour.

Particularly preferred types of brachytherapy radionuclide emitter are as follows: 137-Cs, 60-Co (β⁻ emitters); 192-Ir (γ emitter); 131-Cs, 125-I and 103-Pd (electron capture emitters).

Often, when the internal radiation therapy is brachytherapy, the radioactive material comprises a radioisotope which emits γ-radiation or a radioisotope which emits electrons through β-decay. The radioactive material may for instance comprise a radioisotope which emits γ-radiation. The radioactive material may for instance comprise a radioisotope which emits electrons through β-decay. Usually, therefore, the radioactive material comprises iridium-192 (a gamma emitter) or any of cesium-137, cobalt-60 and yttrium-90 (beta emitters). Alternatively, when the internal radiation therapy is brachytherapy, the radioactive material may comprise a radioisotope which emits photons following electron capture. Thus, the radioactive material may comprise cesium-131, iodine-125 or palladium-103, or a combination of two thereof, or all three of cesium-131, iodine-125 and palladium-103.

Particles of the invention that have a particle size of less than or equal to 100 nm are particularly suitable for use in combination with brachytherapy. Such particles are typically of a size, of equal to or typically less than 100 nm, which permits endocytosis into tumour cells. They may also be coated with silica or organic coatings that enhance steric stabilisation such as PEG, a polyphosphate (e.g. hexametaphosphate) and/or targeting molecules, as defined above, that allow the particles to preferentially interact with tumour cells.

In embodiments of the invention in which the internal radiation therapy is brachytherapy, and especially when the radioactive material comprises a radioisotope which emits γ-radiation or electrons through β-decay, the second semiconductor is often an oxide of gadolinium, europium, erbium, lutetium and/or tungsten. The first semiconductor is often titanium oxide. In other embodiments of the invention in which the internal radiation therapy is brachytherapy, and especially when the radioactive material comprises a radioisotope which emits which emits photons following electron capture, such as cesium-131, iodine-125 or palladium-103, the second semiconductor may be an oxide of zirconium, niobium, tin, molybdenum and ruthenium. These elements advantageously align the K-edge with the emission energy of the electron capture generated photons increasing the absorption of the nanoparticle of the energy and increasing water splitting and free radical generation. The first semiconductor is often titanium oxide.

The radioactive material employed in the internal radiotherapy may comprise radioactive embolization particles. The radioactive embolization particles may for instance be selective internal radiation therapy (SIRT) beads. Embolization starves the site of the cancer of oxygen and generally leads to perfusion-limited hypoxia. This means that the present invention is particularly applicable to the treatment of cancer using radioactive embolization particles such as SIRT beads, due to the fact that particles of the invention facilitate the generation of reactive oxygen species irrespective of the level or presence of molecular oxygen at the site of the cancer.

SIRT beads emit electrons through β-decay of Yttrium-90 at an energy up to 2.28 MeV. These electrons lose energy via the generation of a cascade of photoelectrons until the interaction of an electron with molecular oxygen results in the formation of superoxide radicals and consequent cell death. The interaction volume of 2.28 MeV electrons around the SIRT bead will extend to approximately 10 mm away from the bead. Most tumours have a considerable proportion of hypoxic cells that show resistance to both radiotherapy and chemotherapy. Hypoxia is also associated with an aggressive tumour phenotype and poor prognosis. As the tumour grows oxygen is not able to reach deeper tumour cells by a simple diffusion mechanism. The hypoxic fraction of cells increases with distance from functioning blood vessels—hypoxic cells are present within 20-50 μm of blood vessels but are predominant within 100-150 μm of such vessels. The occlusion of blood vessels with SIRT beads exacerbates the situation as the blood vessel can no longer supply oxygen to surrounding tissue.

Consequently, hypoxic tumour regions lie well within the interaction volume of electrons emitted during β-decay of Yttrium-90. As such the efficacy of cell death induced by the electrons is significantly compromised by the lack of molecular oxygen. The act of blocking the blood vessels by use of embolization beads further compounds the hypoxic issue reducing efficacy of SIRT treatment.

This invention describes a method of overcoming these limitations by the combination of radioactive embolization particles, for instance SIRT beads, with a particle of the invention which acts to scatter electrons and directly generate reactive oxygen species by splitting of water, regardless of the presence of oxygen within tumour tissues.

The particles defined herein, which comprise a first semiconductor and a second semiconductor wherein the first semiconductor forms a heterojunction with the second semiconductor, are used to convert energetic incident electrons into hydroxyl free radicals by a valance band hole mediated water splitting reaction.

h⁺+H₂O→H⁺+OH^{•}

The hole thus generated migrates to the top of the valance band. Owing to the presence of the two semiconductors, the likelihood of electron-hole recombination is minimised and efficiency of radical generation is enhanced. The energy is the converted to hydroxyl free radicals by recombination of the photogenerated hole with external electrons by splitting of water. The hydroxyl free radical generated may damage cellular components via a normal electron exchange interaction.

Particles of the invention of sub-100 nm particle size are particularly effective for use in combination with radioactive embolization particles such as SIRT beads. Such particles are of a size, <100 nm, which permits endocytosis into tumour cells.

Accordingly, the radioactive material employed in the internal radiation therapy may comprise radioactive embolization particles. Typically, the radioactive embolization particles occlude blood vessels which supply the site of the cancer. The site of the cancer typically comprises a tumour, and the radioactive embolization particles typically occlude blood vessels which supply said tumour.

The radioactive embolization particles are typically microparticles. Typically, the radioactive embolization particles have a mean particle size of from 0.1 to 500 μm, for instance from 1 to 500 μm. Typically, the radioactive embolization particles have a mean particle size of from 5 to 200 μm. For instance, the radioactive embolization particles may have a mean particle size of from 5 to 100 μm, for instance from 5 to 90 μm, from 10 to 80 μm, from 10 to 70 μm, or from 20 to 60 μm. Often, the radioactive embolization particles have a mean particle size of from 10 μm to 70 μm, for instance from 20 m to 60 m or from 10 m to 40 μm, for instance from 20 μm to 30 μm.

Typically, the radioactive embolization particles are microspheres. Thus, they typically have a high sphericity. The radioactive embolization particles employed in the present invention may for instance have an average (mean) sphericity of from 0.6 to 1.0, for instance from 0.8 to 1.0.

The radioactive embolization particles typically comprise a radioisotope and a support material. The support material is generally an inert material, for instance a material which is unlikely to react on exposure to ambient conditions or moisture. The inert material is typically biologically inert. Glass, or a polymer or a resin, are commonly employed. The microparticle often comprises greater than 80 wt %, greater than 90 wt % or greater than 95 wt %, of the support material. The radioisotope may be present in elemental form, for instance in the form of particles of the elemental radioisotope dispersed within, or on the surface of, the support material. Alternatively, for instance, the radioisotope may be present in the form of a compound comprising the radioisotope. The compound comprising the radioisotope may, for instance, be impregnated within the support material, or for example coated on the surface of the support material.

The radioisotope employed in the radioactive embolization particles is typically a radioisotope which emits electrons through β-decay. Usually, the radioisotope (which emits electrons through β-decay) is yttrium-90.

In one embodiment, the radioactive embolization particles comprise glass and yttrium-90 and have a particle size of from 10 μm to 40 μm, for instance from 20 μm to 30 μm. Such radioactive embolization particles are commercially available from BTG International under the trade name TheraSphere®.

Alternatively, the radioactive embolization particles may comprise resin and yttrium-90 and have a particle size of from 10 μm to 70 μm, for instance from 20 μm to 60 μm. Such radioactive embolization particles are commercially available from Sirtex under the trade name SIR-Spheres®.

The radioactive embolization particles may be administered to the subject by introducing the radioactive embolization particles directly into said site of the cancer; or by introducing the radioactive embolization particles into the blood stream at a location upstream of said site of the cancer, and allowing the radioactive embolization particles to accumulate at said site of the cancer.

The radioactive embolization particles typically accumulate at the site of the cancer by embolizing blood vessels within the site of the cancer. This both restricts blood flow to the site of the cancer and places the radioactive embolization particles within the site of the cancer in a suitable position to radiotherapeutically treat the cancer. Typically, therefore, the step of administering the radioactive embolization particles comprises parenterally administering the embolization particles into the blood stream of the subject to be treated at a location at or before the site of the cancer. The term “before the site of the cancer” as used herein means upstream in the blood flow from the locus or site of the cancer or tumour tissue, i.e. at a location in the vasculature where blood is flowing towards the site or locus of the cancer or tumour tissue.

The site of the cancer typically comprises a tumour, for instance a tumour comprising a hypoxic region, and the blood stream referred to above is typically arterial tumour vasculature. Accordingly, often, the radioactive embolization particles are administered to the subject by introducing the radioactive embolization particles into arterial tumour vasculature, and allowing the radioactive embolization particles to accumulate in the tumour.

The radioactive embolization particles lodge preferentially in the microvasculature surrounding a tumour, maximising tumoricidal effects and minimising the effects on healthy tissue cells.

The radioactive embolization particles are typically administered to the subject in the form of a composition, i.e. a pharmaceutical composition. Such a pharmaceutical composition typically comprises radioactive embolization particles as defined herein and one or more pharmaceutically acceptable excipients or diluents. The embolization particles are typically administered parenterally, whether subcutaneously, intravenously, intramuscularly, intrasternally, transdermally or by infusion techniques. Thus, the pharmaceutical composition is typically suitable for parenteral administration. Typically, the pharmaceutical composition is suitable for intravenous (including intraarterial) parenteral administration.

Solutions for injection or infusion may contain as diluent, for example, sterile water or typically they may be in the form of sterile, aqueous, isotonic saline solutions. Suspensions and emulsions may contain as an excipient, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable diluent, e.g. sterile water, olive oil, ethyl oleate, glycols, e.g. propylene glycol, and if desired, a suitable amount of lidocaine hydrochloride.

The pharmaceutical composition may comprise a therapeutically effective amount of the radioactive embolization particles. It will be appreciated by one of skill in the art that appropriate dosages of the particles and a pharmaceutical composition comprising the particles can vary from patient to patient. Determining the optimal dosage will generally involve balancing of the level of therapeutic benefit through embolization and release of ROSs against any risk or deleterious side effects. The selected dosage level will depend on a variety of factors including the route of administration, the time of administration, the rate of excretion of the particles, the duration of the treatment, other compounds and/or materials used in combination, the severity of the condition, and the species, sex, age, weight, condition, general health, and prior medical history of the patient. The amount of particles and route of administration will ultimately be at the discretion of the physician, veterinarian, or clinician, although generally the dosage will be selected to achieve local concentrations at the site of action that achieve the desired effect.

Often, the concentration of the radioactive embolization particles in the pharmaceutical composition is from 100 particles/ml to 10¹⁰ particles/ml, for example from 10⁴ particles/ml to 10⁸ particles/ml. Often, the total number of embolization particles in the composition may be from 10 to 10⁶, or from 20 to 10000.

Typically, the concentration of the radioactive embolization particles in the pharmaceutical composition is from 0.05 mg·ml⁻¹ to 50 mg·ml⁻¹, or for instance from 0.1 mg·ml⁻¹ to 20 mg·ml⁻¹, for example from 0.2 mg·ml⁻¹ to 5 mg·ml⁻¹. A concentration of 0.2 mg·ml⁻¹ to 5 mg·ml⁻¹ is in many cases suitable for a typical single dose.

The radioactive embolization particles may be administered together with the particles of the invention in the same composition. Thus, the pharmaceutical composition comprising the radioactive embolization particles may further comprise the particles of the invention as defined herein. The concentration of the particles of the invention in the pharmaceutical composition may, for instance, be from 0.1 mg·ml⁻¹ to 500 mg·ml⁻¹. Usually, for instance, the concentration of the particles of the invention in the pharmaceutical composition is from 0.5 mg·ml⁻¹ to 200 mg·ml⁻¹, for example from 1.0 mg·ml⁻¹ to 100 mg·ml⁻¹. The pharmaceutical composition may for instance comprise from 3 mg·ml⁻¹ to 80 mg·ml⁻¹, or for instance from 5 mg·ml⁻¹ to 60 mg·ml⁻¹, of the particles of the invention.

The treatment of the invention may comprise administering to the subject said pharmaceutical composition of the invention as defined herein which comprises a plurality of particles, wherein the pharmaceutical composition further comprises the radioactive embolization particles. Typically, the concentration of the radioactive embolization particles in the pharmaceutical composition is from 0.05 mg·ml⁻¹ to 50 mg·ml⁻¹, or for instance from 0.1 mg·ml⁻¹ to 20 mg·ml⁻¹, for example from 0.2 mg·ml⁻¹ to 5 mg·ml⁻¹. A concentration of radioactive embolization particles of from 0.2 mg·ml⁻¹ to 5 mg·ml⁻¹ is in many cases suitable for a typical single dose.

A pharmaceutical composition which comprises the plurality of particles of the invention and the radioactive embolization particles is typically administered to the subject by introducing the pharmaceutical composition directly into said site of the cancer; or by introducing the pharmaceutical composition into the blood stream at a location upstream of said site of the cancer, and allowing the radioactive embolization particles and the particles to accumulate at said site of the cancer. As discussed above, the radioactive embolization particles typically accumulate at the site of the cancer by embolizing blood vessels within the site of the cancer. The particles, on the other hand, will accumulate in the site of the cancer—typically in a tumour—passively, via the enhanced permeation and retention mechanism. Alternatively, a targeting moiety may be employed in order to actively target the particles. Typically, therefore, the step of administering the pharmaceutical composition of the invention as defined herein which comprises a plurality of particles of the invention as defined herein and further comprises the radioactive embolization particles, comprises parenterally administering the composition into the blood stream of the subject to be treated at a location at or before the site of the cancer. The term “before the site of the cancer” as used herein means upstream in the blood flow from the locus or site of the cancer or tumour tissue, i.e. at a location in the vasculature where blood is flowing towards the site or locus of the cancer or tumour tissue. Typically, the composition is introduced by catheter or by injection.

Often, in embodiments of the invention in which the radioactive material comprises radioactive embolization particles, the cancer is liver cancer (which may be primary or secondary liver cancer) or renal cancer, i.e. cancer of the kidney. Radioactive embolization particles are particularly suitable for treating such cancers.

In one embodiment the cancer is primary or secondary liver cancer. Typically therefore said site of the cancer comprises a tumour in the liver. Usually, the liver tumour comprises a hypoxic region, i.e. it may contain one or more hypoxic regions.

Typically, in this embodiment, the radioactive embolization particles occlude blood vessels which supply the liver tumour.

Primary and secondary liver tumours derive their blood supply from the hepatic artery whereas approximately 50% of the oxygen supply to the normal liver is via the portal system. Clinical trials of surgery with concurrent chemotherapy showed a ten-fold higher intratumoural concentration when delivered through the hepatic artery than the portal vein. This makes targeting the arterial tumour vasculature attractive since the tumour can be made ischemic (restriction in blood supply) whilst ordinary tissue is spared.

Accordingly, the radioactive embolization particles preferably occlude arterial vasculature in the liver tumour. Preferably, the radioactive embolization particles are administered to the subject by introducing the radioactive embolization particles into the hepatic artery. As discussed above, the particles of the invention may or may not be administered together with the radioactive embolization particles in the same composition.

In another embodiment of the invention in which the radioactive material comprises radioactive embolization particles, the cancer is renal cancer, i.e. cancer of the kidney. Typically therefore said site of the cancer comprises a tumour in the kidney. Usually, in this embodiment, the radioactive embolization particles occlude blood vessels which supply the kidney tumour. The radioactive embolization particles typically occlude arterial vasculature in the kidney tumour. Usually, the kidney tumour comprises a hypoxic region.

Typically, in embodiments of the invention in which the radioactive material comprises radioactive embolization particles, the second semiconductor is often an oxide of gadolinium, lutetium, tungsten, neodymium, europium or erbium. More typically, the second semiconductor is an oxide of gadolinium, lutetium, europium or erbium. The second semiconductor may alternatively comprise tungsten. The first semiconductor, in these embodiments, is often titanium oxide.

The radioactive material employed in the internal radiotherapy may comprise a radiopharmaceutical. Radiopharmaceuticals are a group of drugs which are radioactive. A subset of radiopharmaceuticals are used with therapeutic intent. These are principally used in the palliative treatment of metastatic bone cancers, tumour metastasis to bone being normally considered a terminal event. One key active is 223-Ra dichloride (Xofigo®): an alpha (α-He nucleus) emitter which is injected intravenously. 223-Ra is preferentially absorbed by bone due to its chemical similarity to calcium. Alpha particles are comparatively heavy and charged and as such interact strongly with matter producing large numbers of ions along their path with a corresponding generation of electrons. These electrons can interact with other components generating further electrons and finally superoxide free radicals which damage cancer cells in the immediately vicinity. As alpha particles are so heavy their penetration distance in a solid such as bone is very short—less than 4 m for 5 MeV alpha particles. Another key active is 153-Sm ethylenediaminetetramethylenephosphonic acid (EDTMP) (Quadramet®): a beta (β-650, 710, 810 keV) and gamma (γ-103 keV) emitting radionuclide that shows affinity for bone which concentrates in areas of high bone turnover, such as osteoblastic lesions. Phosphonates show affinity for bone by coordinating calcium ions. Consequently the material targets bone metastases.

A key disadvantage of such treatment however is the requirement for molecular oxygen to be present to allow generation of superoxide free radicals. Hypoxia is a major contributor to tumour metastasis, regulating secreted products that drive tumour-cell proliferation and spread. Hypoxia also contributes to resistance to radiation and chemotherapy in primary tumours. Solid tumours are particularly susceptible to hypoxia because they proliferate rapidly, outgrowing the malformed tumour vasculature, which is unable to meet the increasing metabolic demands of the expanding tumour. This effect is exasperated by bone metastasis since bone is naturally a hypoxic microenvironment capable of potentiating tumour metastasis and growth. Cancer cells capable of surviving at low oxygen levels can thrive in the hypoxic bone microenvironment and participate in the vicious cycle of bone metastasis.

Particles as defined herein, which comprise a first semiconductor and a second semiconductor wherein the first semiconductor forms a heterojunction with the second semiconductor, can be used to enhance free radical generation from electrons produced by inelastic alpha scattering, or from beta or gamma emission, by the water-splitting mechanisms described above. Thus, the particles are used to convert such energetic incident electrons into hydroxyl free radicals by a valance band hole mediated water splitting reaction, thus:

h⁺+H₂O→H⁺+OH^{•}

The hole thus generated migrates to the top of the valance band. Owing to the presence of the two semiconductors, the likelihood of electron-hole recombination is minimised and efficiency of radical generation is enhanced. The energy is the converted to hydroxyl free radicals by recombination of the photogenerated hole with external electrons by splitting of water. The hydroxyl free radical generated may damage cellular components via a normal electron exchange interaction.

Particles of sub-100 nm particle size are particularly effective for use in combination with radiopharmaceuticals such as those described above. Such particles are of a size, <100 nm, which permits endocytosis into tumour cells.

The particles can be used to split water and generate hydroxyl free radicals. The particles can for instance be injected directly into the site of the cancer, for instance into metastatic bone tumours, to enhance the effect of radiopharmaceuticals administered intravenously. Alternatively, the particles and the radiopharmaceutical may be present in the same composition, which may itself be administered directly into the site of the cancer, for instance into metastatic bone tumours.

In this way, radiopharmaceuticals may be combined with the radiosensitising particles of the invention, in a treatment where the particle converts electrons into hydroxyl free radicals, to induce cell death at the site of the cancer. This aspect of the invention is particularly applicable to the treatment of metastatic bone tumours, as well as primary bone tumours such as osteosarcoma, Ewings sarcoma, and chondrosarcoma.

Accordingly, the radioactive material may comprise a radiopharmaceutical. As the skilled person will appreciate, radiopharmaceuticals are a group of pharmaceutical drugs which have radioactivity, and many are known in the art. Radiopharmaceuticals can be used as diagnostic and therapeutic agents. In the present invention, the radioactive material is employed as a therapeutic agent, i.e. for internal radiotherapy, and can therefore be said to comprise a radiopharmaceutical therapeutic agent. As the skilled person will appreciate, a radiopharmaceutical is typically a chemical compound, i.e. a therapeutic agent or drug, which comprises a radioisotope. The compound may be a small molecule drug which comprises a radioisotope or, for instance, a peptide or protein which comprises a radioisotope, for instance a radio-labelled antibody. However, a radiopharmaceutical may alternatively comprise a radioisotope in ionic or elemental form rather than as part of a compound.

The radiopharmaceutical may be administered to the subject by introducing the radiopharmaceutical directly into said site of the cancer; or by administering the radiopharmaceutical systemically.

Thus, performing the internal radiation therapy may further comprise administering the radiopharmaceutical to the subject by: introducing the radiopharmaceutical directly into said site of the cancer; or administering the radiopharmaceutical systemically.

Introducing the radiopharmaceutical directly into said site of the cancer may for instance comprise injecting the radiopharmaceutical directly into said site of the cancer. Thus, a pharmaceutical composition comprising the radiopharmaceutical, may be injected directly into said site of the cancer.

When the site of the cancer comprises a tumour, the radiopharmaceutical may be injected directly into the tumour (i.e. it may be administered by intra-tumoral injection).

Alternatively, introducing the radiopharmaceutical directly into said site of the cancer may comprise introducing the radiopharmaceutical into said site of the cancer, for instance directly into said site of the cancer, via a catheter. Thus, a pharmaceutical composition comprising the radiopharmaceutical may be introduced directly into said site of the cancer via a catheter.

When the site of the cancer comprises a tumour, the radiopharmaceutical may be introduced directly into the tumour via a catheter.

The radiopharmaceutical is typically administered to the subject in the form of a composition, i.e. a pharmaceutical composition. Such a pharmaceutical composition typically comprises a radiopharmaceutical as defined herein and one or more pharmaceutically acceptable excipients or diluents. Solutions for injection or infusion may contain as diluent, for example, sterile water or typically they may be in the form of sterile, aqueous, isotonic saline solutions. Suspensions and emulsions may contain as an excipient, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable diluent, e.g. sterile water, olive oil, ethyl oleate, glycols, e.g. propylene glycol, and if desired, a suitable amount of lidocaine hydrochloride.

In general, the pharmaceutical composition will comprise a therapeutically effective amount of the radiopharmaceutical. It will be appreciated by one of skill in the art that appropriate dosages of the radiopharmaceutical and a pharmaceutical composition comprising the radiopharmaceutical can vary from patient to patient. Determining the optimal dosage will generally involve balancing of the level of therapeutic benefit through the radiopharmaceutical and release of reactive oxygen species (ROS) against any risk or deleterious side effects. The selected dosage level will depend on a variety of factors including the route of administration, the time of administration, the rate of excretion of the radiopharmaceutical, the duration of the treatment, other compounds and/or materials used in combination, the severity of the condition, and the species, sex, age, weight, condition, general health, and prior medical history of the patient. The amount of radiopharmaceutical and route of administration will ultimately be at the discretion of the physician, veterinarian, or clinician, although generally the dosage will be selected to achieve local concentrations at the site of action that achieve the desired effect.

The concentration of the radiopharmaceutical in a pharmaceutical composition used for the administration will of course depend on the particular radiopharmaceutical and the target dose of radiation and can readily be determined by the skilled clinician. The concentration may for instance be selected such that a dose of from 10 to 100 kBq per kg of body weight is achieved in a single injection, for instance a dose of from 35 to 65 kBq per kg of body weight.

Administering the radiopharmaceutical systemically may for instance comprise administering the radiopharmaceutical (or a pharmaceutical composition comprising the radiopharmaceutical as defined above) parenterally. The parenteral administration may for instance be selected from subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid and intrasternal administration. The parenteral administration may be effected, for instance, by injection, or via a catheter. Administering the radiopharmaceutical systemically may for instance comprise administering the radiopharmaceutical or the pharmaceutical composition intravenously, intraarterially, intramuscularly or subcutaneously.

Administering the radiopharmaceutical systemically may alternatively comprise administering the radiopharmaceutical orally, e.g., by ingestion. This would be suitable if a radiopharmaceutical is employed which is adapted for oral administration.

Administering the radiopharmaceutical systemically may further comprise allowing the radiopharmaceutical to accumulate at the site of the cancer. This may comprise allowing the radiopharmaceutical to accumulate at said site by targeting for instance by employing a radiopharmaceutical which comprises a targeting moiety, e.g. a moiety which directs the site specific accumulation of the particles at the target tissue—the site of the cancer, typically a tumour. The targeting moiety may be selected from those listed hereinbefore in connection with the particles employed in the invention. A common targeting moiety for targeting hypoxia, which may for instance be employed in a radiopharmaceutical, is 2-nitroimidazole. Alternatively, a hypoxia-selective radiopharmaceutical may be employed, i.e. a radiopharmaceutical which selectively accumulates in hypoxic tissue. Many hypoxia-selective antitumour agents are known in the art which may be radiolabelled to provide a suitable radiopharmaceutical.

Administering the radiopharmaceutical to the subject may take place either before, during or after administration of the particles. Thus, it may take place either before, during or after delivery of the particles to the site of the cancer.

The radiopharmaceutical may be administered together with the particles of the present invention in the same composition. Thus, the pharmaceutical composition comprising the radiopharmaceutical may further comprise the particles as defined herein. The concentration of the particles in the pharmaceutical composition may, for instance, be from 0.1 mg·ml⁻¹ to 500 mg·ml⁻¹. Usually, for instance, the concentration of the particles in the pharmaceutical composition is from 0.5 mg·ml⁻¹ to 200 mg·ml⁻¹, for example from 1.0 mg·ml⁻¹ to 100 mg·ml⁻¹. The pharmaceutical composition may for instance comprise from 3 mg·ml⁻¹ to 80 mg·ml⁻¹, or for instance from 5 mg·ml⁻¹ to 60 mg·ml⁻¹, of the particles.

The treatment of the invention may comprise administering to the subject said pharmaceutical composition of the invention as defined herein which comprises a plurality of particles, wherein the pharmaceutical composition further comprises the radiopharmaceutical. The pharmaceutical composition will comprise therapeutically effective amounts of the radiopharmaceutical and the particles. As will be appreciated by one of skill in the art, appropriate dosages of the radiopharmaceutical can vary from patient to patient. Determining the optimal dosage will generally involve balancing of the level of therapeutic benefit through the radiopharmaceutical and release of reactive oxygen species (ROS) against any risk or deleterious side effects. The selected dosage level will depend on a variety of factors including the route of administration, the time of administration, the rate of excretion of the radiopharmaceutical, the duration of the treatment, other compounds and/or materials used in combination, the severity of the condition, and the species, sex, age, weight, condition, general health, and prior medical history of the patient. The amount of radiopharmaceutical and route of administration will ultimately be at the discretion of the physician, veterinarian, or clinician, although generally the dosage will be selected to achieve local concentrations at the site of action that achieve the desired effect. The concentration of the radiopharmaceutical in a pharmaceutical composition used for the administration will of course depend on the particular radiopharmaceutical and the target dose of radiation and can readily be determined by the skilled clinician. The concentration may for instance be selected such that a dose of from 10 to 100 kBq per kg of body weight is achieved in a single injection, for instance a dose of from 35 to 65 kBq per kg of body weight. Similar considerations apply to the particles, as discussed hereinbefore. The concentration of the particles of the invention in the pharmaceutical composition which comprises both the radiopharmaceutical and the particles may, for instance, be from 0.1 mg·ml⁻¹ to 500 mg·ml⁻¹. Usually, for instance, the concentration of the particles of the invention in the pharmaceutical composition is from 0.5 mg·ml⁻¹ to 200 mg·ml⁻¹, for example from 1.0 mg·ml⁻¹ to 100 mg·ml⁻¹. The pharmaceutical composition may for instance comprise from 3 mg·ml⁻¹ to 80 mg·ml⁻¹, or for instance from 5 mg·ml⁻¹ to 60 mg·ml⁻¹, of the particles of the invention.

The pharmaceutical composition which comprises a plurality of particles of the invention as defined herein and the radiopharmaceutical may be administered to the subject by: introducing the pharmaceutical composition directly into said site of the cancer; or administering the pharmaceutical composition systemically.

Introducing the pharmaceutical composition directly into said site of the cancer may for instance comprise injecting the pharmaceutical composition directly into said site of the cancer. Thus, a pharmaceutical composition comprising the radiopharmaceutical and the particles, may be injected directly into said site of the cancer. When the site of the cancer comprises a tumour, the pharmaceutical composition may be injected directly into the tumour (i.e. by it may be administered by intra-tumoral injection). Alternatively, introducing the pharmaceutical composition directly into said site of the cancer may comprise introducing the pharmaceutical composition into said site of the cancer, for instance directly into said site of the cancer, via a catheter. Thus, a pharmaceutical composition comprising the radiopharmaceutical and the particles may be introduced directly into said site of the cancer via a catheter. When the site of the cancer comprises a tumour, the pharmaceutical composition may be introduced directly into said tumour via a catheter.

Introducing the pharmaceutical composition systemically may for instance comprise administering the pharmaceutical composition comprising the radiopharmaceutical and the particles parenterally. The parenteral administration may for instance be selected from subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid and intrasternal administration. The parenteral administration may be effected, for instance, by injection, or via a catheter. Administering the radiopharmaceutical systemically may for instance comprise administering the radiopharmaceutical or the pharmaceutical composition intravenously, intraarterially, intramuscularly or subcutaneously. Administering the pharmaceutical composition systemically may alternatively comprise administering the pharmaceutical composition comprising the radiopharmaceutical and the particles orally, e.g., by ingestion.

Administering the radiopharmaceutical systemically may further comprise allowing the radiopharmaceutical and the particles of the invention to accumulate at the site of the cancer. This may comprise allowing the radiopharmaceutical to accumulate at said site by targeting for instance by employing a radiopharmaceutical which comprises a targeting moiety, as described hereinbefore, or a hypoxia-selective radiopharmaceutical. The particles, on the other hand, will accumulate in the site of the cancer—typically in a tumour—passively, via the enhanced permeation and retention mechanism. Alternatively, a targeting moiety may be employed in order to actively target the particles, as described further herein.

The radioactive material may comprise a radioisotope which emits α particles. Alternatively, it may comprise a radioisotope which emits γ-radiation and/or electrons through β-decay.

Thus, in embodiments of the invention in which the radioactive material comprises a radiopharmaceutical, the radiopharmaceutical may comprise a radioisotope which emits α particles. The radioisotope may for instance be 223-Radium (223-Ra). An example of such a radiopharmaceutical is 223-Ra dichloride (Xofigo®).

Alternatively, in embodiments of the invention in which the radioactive material comprises a radiopharmaceutical, the radiopharmaceutical may comprise a radioisotope which emits γ-radiation and/or electrons through β-decay. The radiopharmaceutical may for instance comprise a radioisotope which emits γ-radiation. It may alternatively for instance comprise a radioisotope which emits electrons through β-decay. The radiopharmaceutical may for instance comprise a radioisotope which emits both γ-radiation and electrons through β-decay. The radioisotope may for instance be 153-Samarium (153-Sm), which is a beta (β-650, 710, 810 keV) and gamma (γ-103 keV) emitting radionuclide. An example of such a radiopharmaceutical is 153-Sm ethylenediaminetetramethylenephosphonic acid (EDTMP).

The radioactive material may therefore comprise radium-223 or samarium-153. The radiopharmaceutical may comprise radium-223 or samarium-153. The radiopharmaceutical may for instance be radium-223 dichloride (Xofigo®) or samarium-153 ethylenediaminetetramethylenephosphonic acid (Quadramet®).

Typically, in embodiments of the invention in which the radioactive material comprises a radiopharmaceutical, for instance a radiopharmaceutical as defined above, the cancer is bone cancer or prostate cancer. Typically, therefore, said site of the cancer comprises a bone tumour or a prostate tumour. Usually, the bone tumour or prostate tumour comprises a hypoxic region, i.e. it may contain one or more hypoxic regions.

In one embodiment of the invention in which the radioactive material comprises a radiopharmaceutical, for instance a radiopharmaceutical as defined above, the cancer is bone cancer. The bone cancer may be primary or metastatic. Typically, therefore, said site of the cancer comprises a bone tumour. Usually, the bone tumour comprises a hypoxic region, i.e. it may contain one or more hypoxic regions. The bone tumour may be a primary bone tumour, such as an osteosarcoma, Ewings sarcoma, or chondrosarcoma, or it may be a metastatic bone tumour.

In embodiments of the invention in which the radioactive material employed in the internal radiotherapy comprises a radiopharmaceutical, the radioactive material, i.e. the radiopharmaceutical is typically present within the tumour.

In embodiments of the invention in which the radioactive material employed in the internal radiotherapy comprises a radiopharmaceutical, the second semiconductor in the particles of the invention is typically an oxide of gadolinium, lutetium, tungsten, neodymium, europium or erbium. More typically, the second semiconductor is selected from gadolinium, lutetium, europium and erbium.

As mentioned above, the cancer which is treated in accordance with the present invention comprises a tumour. Thus, the site of the cancer typically comprises a tumour. Often, the tumour comprises a hypoxic region. The tumour may contain one or more hypoxic regions, i.e. it may contain one hypoxic region or it may contain a plurality of hypoxic regions. As the skilled person will understand, not all of the tumour may be hypoxic. Thus, the tumour may comprise a normoxic region, for instance one or more normoxic regions, in addition to the hypoxic region or regions. The tumour may, on the other hand, be entirely hypoxic, i.e. it may not contain any normoxic regions. Thus, the tumour may consist of a hypoxic region.

Tumours are generally known to contain a substantial fraction of cells which are hypoxic. However, in conventional radiotherapy, the concentration of oxygen during, or within milliseconds of irradiation is critical in determining DNA damage and subsequent biological response, with the biological effectiveness of a given dose significantly greater for well-oxygenated cells compared to hypoxic cells. The invention is therefore particularly applicable to the treatment of hypoxic tumours, because the use of the radiosensitising particles of the invention in combination with radiotherapy facilitates the generation of reactive oxygen species directly from water, irrespective of the level or presence of molecular oxygen at the site of the cancer, and thereby increases the efficacy of radiotherapy in hypoxic environments, such as in the treatment of hypoxic tumours. Thus, the invention is particularly applicable to the treatment of cancerous tumours which comprise a hypoxic region, i.e. tumours which contain one or more hypoxic regions.

The term, “hypoxic region”, as used herein, refers to a region within the tumour which comprises hypoxic tumour cells. Areas with very low (down to zero) oxygen partial pressures exist in solid tumours, occurring either acutely or chronically. These microregions of very low or zero O₂ partial pressures are heterogeneously distributed within the tumour mass and may be located adjacent to regions with normal O₂ partial pressures (normoxic regions). Tumour cells which are hypoxic are tumour cells which have a lower concentration of oxygen compared with normoxic cells. Hypoxic tumour cells therefore include anoxic tumour cells, i.e. cells which have an oxygen concentration of substantially 0.0. Usually, the partial pressure of oxygen, pO₂, in a hypoxic cell is at least 3 mmHg below the pO₂ in a normoxic cell, or for instance at least 10 mmHg below the pO₂ in a normoxic cell, e.g. at least 20 mmHg below the pO₂ in a normoxic cell. Often, this results in a pO₂ in the hypoxic cell of less than 50 mmHg, for instance a pO₂ of less than 45 mmHg. The pO₂ in a hypoxic cell may for instance be from 0 to 50 mmHg, for instance from 0 to 45 mmHg, or from 0 to 40 mmHg. More typically, the pO₂ in a hypoxic cell is less than 30 mmHg, for instance less than 20 mmHg, or for instance less than 10 mmHg, for example less than 5 mmHg. A hypoxic cell may for instance have a pO₂ of less than 4 mmHg, for instance less than 2 mmHg, or for example less than 1 mmHg, e.g. less than 0.5 mmHg. The pO₂ in a hypoxic cell may for instance be from 0 to 30 mmHg, for instance from 0 to 20 mmHg, or for instance from 0 to 10 mmHg, for example from 0 to 5 mmHg. A hypoxic cell may for instance have a pO₂ of from 0 to 4 mmHg, for instance from 0 to 2 mmHg, or for instance from 0 to 1 mmHg, for example from 0 to 0.5 mmHg. A “hypoxic region” may therefore be a region within the tumour which comprises hypoxic tumour cells, which hypoxic tumour cells have a pO₂ as defined above. A hypoxic region may consist essentially of hypoxic tumour cells. For instance, a hypoxic region may consist (only) of hypoxic tumour cells. The hypoxic tumour cells may be as further defined above. The hypoxia may be diffusion-limited hypoxia arising from large intervascular distance in the tumour. The hypoxia may be transient ‘acute’ perfusion-limited hypoxia due unstable blood flow in vessels. Perfusion-limited hypoxia may occur due to embolization. It is therefore of significant benefit that the particles of the invention facilitate the generation of reactive oxygen species directly from water, irrespective of the level or presence of molecular oxygen at the site of the cancer.

Any type of cancer can, in principle, be treated. Thus, the invention may for instance be used to treat a cancer of the lung, liver, kidney, bladder, breast, head and neck, oral cavity, throat, pharynx, oropharynx, oesophagus, brain, ovaries, cervix, prostate, intestine, colon, rectum, uterus, pancreas, eye, bone, bone marrow, lymphatic system, connective tissue, non-epithelial tissue or thyroid gland. The cancer may be prostate cancer, liver cancer, renal cancer, bone cancer, bladder cancer, cancer of the oral cavity, throat cancer, oropharyngeal cancer, sarcoma, lung cancer, cervical cancer, oesophageal cancer, breast cancer, brain cancer, ovarian cancer, intestinal cancer, bowel cancer, colon cancer, rectal cancer, uterine cancer, pancreatic cancer, eye cancer, lymphoma, or thyroid cancer. The bone cancer may be primary or metastatic. Typically, the invention may be used to treat a cancer of the pancreas, head and neck, lung, bladder, breast, oesophagus, stomach, liver, salivary glands, kidney, prostate, cervix, ovaries, soft tissue sarcomas, melanoma, brain, bone or metastatic tumours arising from any primary tumour.

In some cases, the particle or pharmaceutical composition may be used to treat a cancer of a radiosensitive organ. In such instances, the cancer may be a cancer of the salivary glands, liver, stomach, spinal column, lymph nodes, reproductive organs or digestive organs.

As part of the therapy or treatment or cancer, particles of the invention as defined herein, whether as part of a pharmaceutical composition of the invention, combination product or otherwise, may be administered to a subject by any convenient route of administration. Thus, any reference to the treatment of cancer in combination with radiotherapy generally refers to the treatment of cancer by administering to a subject a particle or particles as defined herein, whether as a pharmaceutical composition, combination, product or otherwise, and then irradiating a site of the cancer via radiotherapy.

Typically, the radiotherapy comprises irradiating a site of the cancer with radiation from an external source or from a radioactive material inside the subject. As the skilled person will appreciate, the treatment generally comprises irradiating a site of the cancer at which the particle or particles are present. The treatment typically therefore comprises performing radiotherapy on a site of the cancer to which the particle or particles have been delivered.

Thus, in the treatment of the invention, a particle as defined herein, or a pharmaceutical composition as defined herein which comprises a plurality of the particles, is typically administered to the subject. The treatment also typically comprises delivering the particle or particles to the site of the cancer. Thus, the treatment may comprise delivering the particle (or particles) to the site of the cancer and performing radiotherapy.

Administering the particle or the pharmaceutical composition to the subject typically comprises (a) introducing the particle or the pharmaceutical composition directly into said site of the cancer, or (b) administering the particle or the pharmaceutical composition systemically. Administering the particle or the pharmaceutical composition systemically typically further comprises allowing the particle or particles to accumulate at the site of the cancer.

Introducing the particle or the pharmaceutical composition directly into said site of the cancer may for instance comprise injecting the particle or the pharmaceutical composition directly into said site of the cancer. When the site of the cancer comprises a tumour, an intra-tumoral injection may be performed. Alternatively, introducing the particle or the pharmaceutical composition directly into said site of the cancer may comprise introducing the particle or the pharmaceutical composition into said site of the cancer via a catheter.

Administering the particle or the pharmaceutical composition may for instance comprise administering the particle or the pharmaceutical composition topically, i.e. the composition may be applied to a particular place on or in the body. In this embodiment the composition is generally administered topically (applied) to a site of the cancer which may be as further defined herein. Thus, administering the pharmaceutical composition to the subject may comprise topical administration of the pharmaceutical composition onto a site of the cancer. The site of a cancer typically, in this embodiment, comprises a region of unresected tumour following surgery. The pharmaceutical composition employed is one which is suitable for topical administration, for instance a gel, cream, paint or spray comprising said plurality of particles. A composition suitable for topical administration, such as a gel, cream, spray or paint comprising the particles, may be applied directly to a site of a cancer prior to radiotherapy. Topical administration is particularly suitable when the site of the cancer is a region of unresected tumour following surgery. In this case, the cancer may for example be a cancer of the bowel, colon, rectum or brain.

Administering the particle or the pharmaceutical composition systemically may for instance comprise administering the particle or the pharmaceutical composition parenterally. The parenteral administration may for instance be selected from subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, topical and intrasternal administration. The parenteral administration may be effected, for instance, by injection, or via a catheter. Administering the particle or the pharmaceutical composition systemically may for instance comprise administering the particle or the pharmaceutical composition intravenously, intraarterially, intramuscularly or subcutaneously.

Alternatively, administering the particle or the pharmaceutical composition systemically may comprise administering the particle or the pharmaceutical composition orally, e.g., by ingestion.

Allowing the particle(s) to accumulate at the site of the cancer may comprise allowing the particle(s) to accumulate at said site by passive targeting or active targeting. Typically, the site of the cancer comprises a tumour. Thus, allowing the particle(s) to accumulate at the site of the cancer may comprise allowing the particle(s) to accumulate at said tumour. This may be by passive targeting or active targeting

The first mechanism, so-called passive targeting, is non-specific and relies on the accumulation of the particles at the site of the cancer, e.g. in a tumour at the site. The particles employed in the present invention are capable of accumulating at the site of the cancer (e.g. in the tumour) passively, via the enhanced permeation and retention mechanism.

The second mechanism is a process of active targeting where a targeting moiety (e.g. a ligand) directs the site specific accumulation of the particles at the target tissue—the site of the cancer, typically a tumour. This may be achieved by attaching or conjugating to the particles a targeting moiety that possesses a high affinity for a molecular signature or structure found predominantly or exclusively in the malignant cells. The targeting moiety has a preferential binding affinity for a biological moiety, such as a molecular signature or structure (e.g. a gene, a protein, an organelle, such as mitochondria), which is generally only present in a cancer cell or a tumour tissue. The targeting moiety is capable of concentrating the particles in the tumour tissue or cancer cells. A particle as defined herein may therefore comprise at least one targeting moiety. A targeting moiety may be attached to a coating of a particle, for instance a silica coating disposed on the surface of the nanoparticle, as described in International patent application no. PCT/GB2010/002247 (WO 2011/070324). The targeting moiety may be a peptide, a polypeptide, a nucleic acid, a nucleotide, a lipid, a metabolite, an antibody, a receptor ligand, a ligand receptor, a hormone, a sugar, an enzyme, a vitamin or the like. For example, the targeting moiety may be selected from a drug (e.g. trastuzumab, gefitinib, PSMA, tamoxifen/toremifen, imatinib, gemtuzumab, rituximab, alemtuzumab, cetximab), a DNA topoisomerase inhibitor, an antimetabolite, a disease cell cycle targeting compound, a gene expression marker, an angiogenesis targeting ligand, a tumour marker, a folate receptor targeting ligand, an apoptotic cell targeting ligand, a hypoxia targeting ligand, a DNA intercalator, a disease receptor targeting ligand, a receptor marker, a peptide (e.g. a signal peptide, a melanocyte stimulating hormone (MSH) peptide), a nucleotide, an antibody (e.g. an antihuman epidermal growth factor receptor 2 (HER2) antibody, a monoclonal antibody C225, a monoclonal antibody CD31, a monoclonal antibody CD40), an antisense molecule, an siRNA, a glutamate pentapeptide, an agent that mimics glucose, amifostine, angiostatin, capecitabine, deoxycytidine, fullerene, herceptin, human serum albumin, lactose, quinazoline, thalidomide, transferrin and trimethyl lysine. Typically, the targeting moiety is a nuclear localization signal (NLS) peptide.

Accordingly, the particle employed in the present invention, or each particle in the plurality of particles employed in the invention, may further comprise a targeting moiety. The targeting moiety may be attached or conjugated to the or each nanoparticle, for instance to the surface of the or each nanoparticle, or to a coating on the surface of the or each nanoparticle.

The particle employed in the present invention, or each particle in the plurality of particles employed in the invention, may further comprise a coating. The coating may be a coating of one or more compounds selected from silica, alumina, or an organic coating, for instance polyethylene glycol, polystyrene, a saccharide, an oligosaccharide, a polysaccharide, a polyvinylpyrrolidone, or a polyphosphate or mixtures of two or more of such compounds. The coating may be an organic coating, such as PEG, that enhances steric stabilisation. The coating may be a negatively charged coating such as a polyphosphate, for instance hexametaphosphate, that enhances cellular uptake. The inclusion of a coating on the particles can improve their biocompatibility, prevent them from agglomerating in vivo and allow them to be functionalised with other agents, for instance with one or more targeting moieties as described above. Any reference to the particle size of a particle, as employed in the invention, refers to the total size of the particle, including any coating that may be present. When there is a plurality of particles such that the size is an average particle size, then the size refers to the average total size, including any coating(s) that may be present, of the particles. In general, the thickness of the coating is from 0.1 to 10 nm, typically from 1 to 5 nm. It is preferred that the coating is silica or an organic coating (for instance PEG, sucrose or a polyphosphate). Typically, the coating is silica. More typically, the particle or particles comprise a silica coating with a thickness of less than 5 nm.

Generally, in the treatment of the invention, a therapeutically effective amount of the particles, whether as a pharmaceutical composition, combination, product or otherwise, are administered to a subject. Administration can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target tissue or cells being treated, the subject being treated, and the particular radiotherapy being employed. Single or multiple administrations of the particles can be carried out with the dose level and pattern being selected by the treating physician, veterinarian, or clinician.

As explained above, typically, the particles employed in the invention enhance the effect of radiotherapy in the treatment of a cancer, by overcoming a particular limitation of conventional radiotherapy, namely that, in order for such therapy to be effective an adequate level of molecular oxygen needs to be present in the cancerous tissue being treated. Thus, the invention relates to the use of the particles, whether as part of a pharmaceutical composition, combination, product, medicament or otherwise, as a radiosensitizing agent, in the treatment of cancer when used in combination with radiotherapy. A radiosensitizing agent can allow the dosage of the radiation to be reduced without a loss of efficacy, such that a similar therapeutic outcome is obtained compared to that obtained from using higher doses of the radiation in the absence of the particles of the invention. Alternatively, the radiosensitizing agent improves the effect of the radiation, which results in an improved therapeutic outcome for the patient compared to that obtained when using the same dose of the radiation in the absence of the particles employed the present invention.

Administration and delivery of the particles to the site of the cancer may take place before, during or after the commencement of the radiotherapy. In some embodiments it is preferred that the particles are already in place when radiotherapy is commenced, and the particles are therefore delivered before the radiotherapy is administered to the subject.

Alternatively, the particles can be administered at the same time as, or even after, the radiotherapy is commenced, provided of course that the particles are delivered to the site of the cancer at some point during the radiotherapy, so that the particles are able to enhance the effect of the radiotherapy in accordance with the invention.

The treatment of the invention may further comprise detecting the presence or absence of the particle or pharmaceutical composition at a site of the cancer before performing radiotherapy. Typically, the step of detecting the presence or absence of the particle or particles at a site of a cancer comprises directing X-rays at the site to obtain an X-ray image. The X-ray image may then be used to determine if a cancer or tumour tissue is present or absent at the site and also whether the particle or pharmaceutical composition is present at the site. For diagnostic uses, the exposure time of a subject to X-rays is generally from one second to 30 minutes, typically from one minute to 20 minutes and more typically from one second to 5 minutes.

If the particle or particles comprises an optical contrast agent, a radioisotope, a paramagnetic contrast agent or a superparamagnetic contrast agent, then the agent may be used to perform the step of detecting the presence or absence of the particle or particles at the site. The exact method of detecting the particle or particles will depend on the optical contrast agent, radioisotope, paramagnetic contrast agent or superparamagnetic contrast agent that is present. The contrast agent may be a gadolinium MRI contrast agent.

In some cases, i.e. when external radiotherapy is employed, the treatment comprises delivering the particle (or particles) to a site of the cancer and irradiating the site of the cancer with radiation from an external source (external radiotherapy). The radiotherapy may be selected from conformal radiotherapy, intensity modulated radiotherapy (IMRT), image guided radiotherapy (IGRT), 4-dimensional radiotherapy (4D-RT), stereotactic radiotherapy and radiosurgery, proton therapy, electron beam radiotherapy, and adaptive radiotherapy.

Thus, typically, the external radiotherapy comprises irradiating a site of the cancer with radiation from an external source. The radiation is generally directed to said site of the cancer. As the skilled person will appreciate, the treatment generally comprises performing external radiotherapy on a site of the cancer at which the particle is present. In other words, the treatment generally comprises irradiating a site of the cancer at which the particle is present with radiation from an external source. The treatment typically therefore comprises performing external radiotherapy on a site of the cancer to which the particle has been delivered. Thus, the treatment typically comprises irradiating a site of the cancer to which the particle has been delivered with radiation from an external source.

Accordingly, administration and delivery of the particles to the site of the cancer may take place before, during or after the external radiotherapy is administered to the subject. It is preferred that the particles are already in place when the external radiotherapy is administered to the subject. Alternatively, the particles can be administered at the same time as the external radiotherapy is administered.

In other cases, i.e. when internal radiotherapy is employed, the treatment comprises delivering the particle (or particles) to a site of the cancer and irradiating the site of the cancer with radiation from a radioactive material inside the subject. The radiotherapy may be brachytherapy or the radioactive material inside the subject may comprise radioactive embolization particles or a radiopharmaceutical, as discussed herein.

Typically, the treatment further comprises administering the radioactive material to the subject. Administering the radioactive material to the subject may comprise: (i) introducing the radioactive material directly into or near to said site of the cancer; or (ii) administering the radioactive material systemically and allowing the radioactive material to accumulate at said site of the cancer. The term “near to” in this context, means that the radioactive material should be near enough to the site of the cancer for the radiation from the radioactive material to be able to reach the site of the cancer and thereby treat the cancer effectively in accordance with the present invention. For example, in brachytherapy, the radiation source may be implanted into the patient adjacent the site of the cancer (for instance adjacent a cancerous tumour) rather than actually be embedded within it. Accordingly, “near to said site of the cancer” typically means “adjacent to” or “next to” the site of the cancer.

Introducing the radioactive material directly into or near to said site of the cancer may for instance comprise injecting the radioactive material directly into or near to said site of the cancer, for instance injecting the radioactive material directly into said site of the cancer. For instance, when radioactive embolization particles are, or when a radiopharmaceutical is, employed as the radioactive material, the radioactive material may be injected directly into or near to said site of the cancer. Thus, a pharmaceutical composition comprising the radioactive embolization particles, or a pharmaceutical composition comprising the radiopharmaceutical, may be injected directly into or near to said site of the cancer.

When the site of the cancer comprises a tumour, the radioactive material may be injected directly into the tumour (i.e. by it may be administered by intra-tumoral injection).

Alternatively, introducing the radioactive material directly into or near to said site of the cancer may comprise introducing the radioactive material into or near to said site of the cancer, for instance directly into said site of the cancer, via a catheter. For instance, when radioactive embolization particles are, or when a radiopharmaceutical is, employed as the radioactive material, the radioactive material may be introduced directly into or near to said site of the cancer via a catheter. Thus, a pharmaceutical composition comprising the radioactive embolization particles, or a pharmaceutical composition comprising the radiopharmaceutical, may be introduced directly into or near to said site of the cancer via a catheter.

When the site of the cancer comprises a tumour, the radioactive material may be introduced directly into the tumour via a catheter.

Alternatively, introducing the radioactive material directly into or near to said site of the cancer may comprise implanting the radioactive material into the subject, either into or near to said site of the cancer, for instance into the site of the cancer. When the site of the cancer comprises a tumour, the radioactive material may be implanted into or near to the tumour, for instance adjacent to the tumour or inside the tumour. This approach, of implanting the radioactive material into the subject, either into or near to said site of the cancer, is typically employed for brachytherapy.

Administering the radioactive material systemically may for instance comprise administering the radioactive material parenterally. The parenteral administration may for instance be selected from subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid and intrasternal administration. The parenteral administration may be effected, for instance, by injection, or via a catheter. Administering the radioactive material systemically may for instance comprise administering the nanoparticle or the pharmaceutical composition intravenously, intraarterially, intramuscularly or subcutaneously.

Administering the radioactive material systemically may alternatively comprise administering the radioactive material orally, e.g., by ingestion. This may for instance be suitable if a radiopharmaceutical is employed which is adapted for oral administration.

Allowing the radioactive material to accumulate at the site of the cancer may comprise allowing the radioactive material to accumulate at said site by passive targeting or active targeting.

The first mechanism, so-called passive targeting, is non-specific and relies on the accumulation of the radioactive material at the site of the cancer, for instance in a tumour tissue. In the case of radioactive embolization particles, the particles typically accumulate at the site of the cancer by embolizing vasculature within the site of the cancer. This both restricts blood flow to the site of the cancer and places the embolization particles within the site of the cancer in a suitable position to radiotherapeutically treat the cancer by internal radiation therapy. Thus, the treatment of cancer in combination with internal radiation therapy, in accordance with the present invention, may comprise a step of allowing radioactive embolization particles to embolize vasculature within the site of the cancer. In cases where the radioactive material comprises a radiopharmaceutical, the radiopharmaceutical may be one which selectively accumulates in cancerous tissue. For instance, it may be a hypoxic selective radiopharmaceutical, i.e. one which preferentially accumulates in hypoxic tissue as opposed to normoxic tissue.

The second mechanism is a process of active targeting where a targeting moiety (e.g. a ligand) directs the site-specific accumulation of the radioactive material at the target tissue. This may be achieved by attaching or conjugating to the radioactive material (e.g. to radioactive embolization particles or to a radiopharmaceutical) a targeting moiety that possesses a high affinity for a molecular signature or structure found predominantly or exclusively in the malignant cells. The targeting moiety has a preferential binding affinity for a biological moiety, such as a molecular signature or structure (e.g. a gene, a protein, an organelle, such as mitochondria), which is generally only present in a cancer cell or a tumour tissue. The targeting moiety is capable of concentrating the radioactive material at the site of the cancer, e.g. in tumour tissue or cancer cells. A radioactive material as defined herein, particularly a radiopharmaceutical or radioactive embolization particles, may therefore comprise a targeting moiety. The targeting moiety may be as defined above in connection with the particles of the invention. Indeed, the particle(s) and the radioactive material may comprise the same targeting moiety, in order that they are both targeted to the same site.

Thus, typically, the internal radiation therapy comprises irradiating a site of the cancer with radiation from a radioactive material inside the subject. The radioactive material is generally located at or near to said site of the cancer, typically at the site of the cancer. As the skilled person will appreciate, the internal radiation therapy treatment generally comprises performing internal radiation therapy on a site of the cancer at which the particle is present. In other words, the treatment generally comprises irradiating a site of the cancer at which the particle is present with radiation from a radioactive material inside the subject. The treatment typically therefore comprises performing internal radiation therapy on a site of the cancer to which the particle has been delivered. Thus, the treatment typically comprises irradiating a site of the cancer to which the particle has been delivered with radiation from a radioactive material inside the subject.

Accordingly, administration and delivery of the particles to the site of the cancer may take place before, during or after the radioactive material which is employed in the internal radiation therapy is administered to the subject. In some embodiments it is preferred that the particles are already in place when the radioactive material is administered to the subject. Alternatively, the particles can be administered at the same time as, or after, the radioactive material is administered. An example of administration at the same time is when the treatment comprises administering to the subject a composition comprising both (i) particles as defined herein and (ii) a radioactive material suitable for internal radiation therapy, i.e. if the radioactive material is in the same composition as the particles. This may well be the case for example if radioactive embolization particles are employed, or if a radiopharmaceutical is employed, as the radioactive material.

The treatment of the cancer may be multimodal. For instance, the treatment of the cancer may be further combined with other treatments such as chemotherapy or immunotherapy.

Chemotherapy has been used synergistically with radiotherapy for many years in neoadjuvant, adjuvant and concurrent settings. Concurrent chemotherapy capitalises on the radiosensitising properties of many chemotherapy drugs, for example cisplatin and 5-fluorouracil, to deliver treatment benefits beyond those achieved by either chemotherapy or radiotherapy alone. However, the radiosensitising properties of intravenously administered chemotherapy drugs are not tumour specific and also affect adjacent normal tissue within the radiation field. Consequently concurrent chemotherapy trials have consistently reported an increase in acute, severe and life threatening grade 3 and 4 toxic events. A combination of the current invention with chemotherapy would allow radiotherapy dose to be reduced during treatment reducing side effects whilst maintaining efficacy. The chemotherapeutic agent may be selected from cisplatin, carboplatin, toxoids including paclitaxel and docetaxel, 5-fluorouracil, vinca alkaloids including vinorelbine, and gemcitabine. Chemotherapy may be performed before, during or after the radiotherapy. The chemotherapy generally comprises administering a chemotherapeutic agent to the subject. The chemotherapy may comprise administering a chemotherapeutic agent systemically, or administering a chemotherapeutic agent locally to a site of the cancer.

The treatment of the invention may further comprise chemotherapy. This may be neoadjuvant chemotherapy, concurrent chemotherapy or adjuvant chemotherapy. In other words, the treatment of the invention may further comprise neoadjuvant, concurrent or adjuvant dosing of a chemotherapeutic agent.

Accordingly, in one embodiment, the invention provides a particle of the invention or a pharmaceutical composition of the invention for use in combination with radiotherapy in the treatment of cancer in a subject, wherein said treatment of the cancer further comprises chemotherapy. The particle and the treatment may be as further defined anywhere herein.

A pharmaceutical composition comprising a plurality of the nanoparticles is typically employed, and therefore the invention also provides a pharmaceutical composition of the invention for use in combination with radiotherapy in the treatment of cancer, wherein said treatment of the cancer further comprises chemotherapy. The pharmaceutical composition of the invention and the treatment may be as further defined anywhere herein. The pharmaceutical composition may further comprise a chemotherapeutic agent. The chemotherapeutic agent may be as further defined hereinbelow.

The chemotherapy may be performed before, during or after the radiotherapy. The chemotherapy generally comprises administering a chemotherapeutic agent to the subject. The chemotherapeutic agent may be administered systemically or locally to a site of the cancer. The site of the cancer may be the same site of the cancer, referred to elsewhere herein, which is irradiated with the radiation which is employed in the radiotherapy.

Accordingly, in one embodiment the chemotherapy is performed before the radiotherapy. Thus, the chemotherapy may be neoadjuvant chemotherapy. The chemotherapeutic agent may be administered systemically or locally as discussed above.

In another embodiment the chemotherapy is performed during the radiotherapy. Thus, the chemotherapy may be concurrent chemotherapy. The chemotherapeutic agent may be administered systemically or locally as discussed above.

In another embodiment the chemotherapy is performed after the radiotherapy. Thus, the chemotherapy may be adjuvant chemotherapy. The chemotherapeutic agent may be administered systemically or locally as discussed above.

The chemotherapeutic agent may be any anticancer drug, or any combination of anticancer drugs, which is suitable for treating the cancer in question. Such agents are well known. The chemotherapeutic agent may for instance be Abiraterone Acetate, Abitrexate (Methotrexate), Abraxane (Paclitaxel Albumin-stabilized Nanoparticle Formulation), ABVD (i.e. a combination of Doxorubicin Hydrochloride (Adriamycin), Bleomycin, Vinblastine Sulfate and Dacarbazine), ABVE (i.e. a combination of Doxorubicin Hydrochloride, Bleomycin, Vincristine Sulfate and Etoposide), ABVE-PC (i.e. a combination of Doxorubicin Hydrochloride, Bleomycin, Vincristine Sulfate, Etoposide, Prednisone and Cyclophosphamide), AC (i.e. a combination of Doxorubicin Hydrochloride and Cyclophosphamide), AC-T (i.e. a combination of Doxorubicin Hydrochloride, Cyclophosphamide and Paclitaxel (Taxol)), Adcetris (Brentuximab Vedotin), ADE (i.e. a combination of Cytarabine (Ara-C), Daunorubicin Hydrochloride and Etoposide), Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran for Injection (Melphalan Hydrochloride), Alkeran Tablets (Melphalan), Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Axitinib, Azacitidine, BEACOPP (i.e. a combination of Bleomycin, Etoposide, Doxorubicin Hydrochloride, Cyclophosphamide, Vincristine Sulfate (Oncovin), Procarbazine Hydrochloride and Prednisone), Becenum (Carmustine), Beleodaq (Belinostat), Belinostat, Bendamustine Hydrochloride, BEP (i.e. a combination of Bleomycin, Etoposide and Cisplatin (Platinol)), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF (i.e. a combination of Cyclophosphamide, Doxorubicin Hydrochloride (Adriamycin) and Fluorouracil), Campath (Alemtuzumab), Camptosar (Irinotecan Hydrochloride), Capecitabine, CAPOX (a combination of Capecitabine and Oxaliplatin), Carac (Fluorouracil-Topical), Carboplatin, CARBOPLATIN-TAXOL (a combination of Carboplatin and Paclitaxel), Carfilzomib, Carmubris (Carmustine), Carmustine, Carmustine Implant, Casodex (Bicalutamide), CEM (a combination of Carboplatin, Etoposide and Melphalan Hydrochloride), Ceritinib, Cerubidine (Daunorubicin Hydrochloride), Cervarix (Recombinant HPV Bivalent Vaccine), Cetuximab, Chlorambucil, CHLORAMBUCIL-PREDNISONE (a combination of Chlorambucil and Prednisone), CHOP (a combination of Cyclophosphamide, Doxorubicin Hydrochloride (Hydroxydaunomycin), Vincristine Sulfate (Oncovin) and Prednisone), Cisplatin, Clafen (Cyclophosphamide), Clofarabine, Clofarex (Clofarabine), Clolar (Clofarabine), CMF (a combination of Cyclophosphamide, Methotrexate and Fluorouracil), Cobimetinib, Cometriq (Cabozantinib-S-Malate), COPDAC (a combination of Cyclophosphamide, Vincristine Sulfate (Oncovin), Prednisone and Dacarbazine), COPP (a combination of Cyclophosphamide, Vincristine Sulfate (Oncovin), Procarbazine Hydrochloride and Prednisone), COPP-ABV (a combination of Cyclophosphamide, Vincristine Sulfate, Procarbazine Hydrochloride, Prednisone, Doxorubicin Hydrochloride, Bleomycin and Vinblastine Sulfate), Cosmegen (Dactinomycin), Cotellic (Cobimetinib), Crizotinib, CVP (a combination of Cyclophosphamide, Vincristine Sulfate and Prednisone), Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH (a combination of Etoposide, Prednisone, Vincristine Sulfate, Cyclophosphamide and Doxorubicin Hydrochloride), Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC (a combination of Fluorouracil, Epirubicin Hydrochloride, and Cyclophosphamide), Femara (Letrozole), Filgrastim, Fludara (Fludarabine Phosphate), Fludarabine Phosphate, Fluoroplex (Fluorouracil-Topical), Fluorouracil Injection Fluorouracil-Topical, Flutamide, Folex (Methotrexate), Folex PFS (Methotrexate), FOLFIRI (a combination of Leucovorin Calcium (Folinic Acid), Fluorouracil and Irinotecan Hydrochloride), a combination of 5-fluorouracil, oxaliplatin and folinic acid (as used in FOXFIRE), FOLFIRI-BEVACIZUMAB (a combination of Leucovorin Calcium, Fluorouracil, Irinotecan Hydrochloride and Bevacizumab), FOLFIRI-CETUXIMAB (a combination of Leucovorin Calcium, Fluorouracil, Irinotecan Hydrochloride and Cetuximab), FOLFIRINOX (a combination of Leucovorin Calcium, Fluorouracil, Irinotecan Hydrochloride and Oxaliplatin), FOLFOX (a combination of Leucovorin Calcium, Fluorouracil and Oxaliplatin), Folotyn (Pralatrexate), FU-LV (a combination of Fluorouracil and Leucovorin Calcium), Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin combination, Gemcitabine-Oxaliplatin combination, Gemtuzumab, Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine Recombinant, HPV Nonavalent Vaccine Recombinant, HPV Quadrivalent Vaccine Recombinant, Hycamtin (Topotecan Hydrochloride), Hydrea (Hydroxyurea), Hydroxyurea, Hyper-CVAD (a combination of Cyclophosphamide, Vincristine Sulfate, Doxorubicin Hydrochloride (Adriamycin) and Dexamethasone), Ibrance (Palbociclib), Ibritumomab Tiuxetan, Ibrutinib, ICE (a combination of Ifosfamide, Carboplatin and Etoposide), Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan, Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP (a combination of Mechlorethamine Hydrochloride, Vincristine Sulfate (Oncovin), Procarbazine Hydrochloride and Prednisone), Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neupogen (Filgrastim), Nexavar (Sorafenib Tosylate), Nilotinib, Ninlaro (Ixazomib Citrate), Nivolumab, Nolvadex (Tamoxifen Citrate), Nplate (Romiplostim), Obinutuzumab, Odomzo (Sonidegib), OEPA (a combination of Vincristine Sulfate, Etoposide, Prednisone and Doxorubicin Hydrochloride), Ofatumumab, OFF (a combination of Oxaliplatin, Fluorouracil, Leucovorin Calcium (Folinic Acid)), Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA (a combination of Vincristine Sulfate (Oncovin), Procarbazine Hydrochloride, Prednisone and Doxorubicin Hydrochloride (Adriamycin)), Osimertinib, Oxaliplatin, Paclitaxel, Paclitaxel Albumin-stabilized Nanoparticle Formulation, PAD (a combination of Bortezomib (PS-341), Doxorubicin Hydrochloride (Adriamycin) and Dexamethasone), Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV (a combination of Procarbazine Hydrochloride, Lomustine (CCNU) and Vincristine Sulfate), Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Raloxifene Hydrochloride, Ramucirumab, Rasburicase, R-CHOP (a combination of Rituximab, Cyclophosphamide, Doxorubicin Hydrochloride, Vincristine Sulfate, and Prednisone), R-CVP (a combination of Rituximab, Cyclophosphamide, Vincristine Sulfate and Prednisone), Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH (a combination of Rituximab, Etoposide, Prednisone, Vincristine Sulfate, Cyclophosphamide and Doxorubicin Hydrochloride), Revlimid (Lenalidomide), Rheumatrex (Methotrexate), Rituxan (Rituximab), Rituximab, Rolapitant Hydrochloride, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V (a combination of Mechlorethamine Hydrochloride, Doxorubicin Hydrochloride, Vinblastine Sulfate, Vincristine Sulfate, Bleomycin, Etoposide and Prednisone), Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), TAC (a combination of Docetaxel (Taxotere), Doxorubicin Hydrochloride (Adriamycin) and Cyclophosphamide), Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF (Docetaxel (Taxotere), Cisplatin (Platinol) and Fluorouracil), Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC (a combination of Vincristine Sulfate, Dactinomycin (Actinomycin-D) and Cyclophosphamide), Vandetanib, VAMP (Vincristine Sulfate, Doxorubicin Hydrochloride (Adriamycin), Methotrexate and Prednisone), Varubi (Rolapitant Hydrochloride), Vectibix (Panitumumab), VeIP (a combination of Vinblastine Sulfate (Velban), Ifosfamide and Cisplatin (Platinol)), Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP (a combination of Etoposide (VePesid), Ifosfamide and Cisplatin (Platinol)), Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI (a combination of Capecitabine (Xeloda) and Irinotecan Hydrochloride), XELOX (Capecitabine (Xeloda) and Oxaliplatin), Xgeva (Denosumab), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) or Zytiga (Abiraterone Acetate). The chemotherapeutic agent may be selected from any one of the aforementioned agents, or a combination of two or more of any of the aforementioned agents may be employed, for instance a combination of any two, three, four, five, six, seven or eight of the aforementioned agents may be employed, as the chemotherapeutic agent.

The chemotherapeutic agent may for instance be a combination of 5-fluorouracil, oxaliplatin and folinic acid (as used in FOXFIRE). This is particularly preferable when the radiotherapy is internal radiotherapy and the radioactive material employed for the internal radiation therapy comprises radioactive embolization particles, for instance SIRT beads. This is also particularly preferable when the cancer being treated is liver cancer or cancer of the kidney. It is particularly useful when the cancer being treated is liver cancer, and for instance comprises primary or metastatic liver tumours.

The chemotherapeutic agent may be administered to the subject by Transcatheter Arterial Chemoembolization (TACE) using Drug Eluting Beads, i.e. by TACE beads. In this embodiment, the chemotherapeutic agent, which may be any of the chemotherapeutic agents defined herein, is delivered to the site of the cancer (for instance to a tumour) by drug-eluting beads. The drug eluting beads are typically microparticles, more typically microspheres, as defined herein. The microparticles or microspheres are typically biocompatible, non-resorbent and contain a chemotherapy agent which may be any of the chemotherapeutic agents defined herein, and may, for instance, be Doxorubicin or Irinotecan. Administration by TACE beads is preferable when the radioactive material employed for the internal radiation therapy comprises radioactive embolization particles, for instance SIRT beads. This is also particularly preferable when the cancer being treated is liver cancer or cancer of the kidney. It is particularly useful when the cancer being treated is liver cancer, and for instance comprises primary or metastatic liver tumours.

The chemotherapeutic agent may be administered systemically or locally. Local delivery may for instance be by TACE beads or biodegradable beads which contain the chemotherapeutic agent, or by any other type of localised drug delivery.

Recently, it has been demonstrated that combining radiotherapy and immunotherapy can lead to more efficacious cancer treatment than either therapy alone. Radiotherapy can activate the immune system by inducing localised cell death, resulting in production and release of cytokines and chemokines into the tumour microenvironment. This leads to the infiltration of cytotoxic T-cells into the tumour and consequent stimulation of the immune system to attack the tumour. In fact, this immune stimulating effect of radiotherapy can even lead to tumour responses in off target metastases (known as the abscopal effect). In cancer the normal immune system response is inhibited or deregulated, allowing cancer cells to escape from the immune system and survive. Immune system T cells can recognise and destroy cancer cells, however this inhibitory mechanism prevents them doing so. Overcoming this inhibition is the basis of immunotherapy. Over the past few years numerous clinical trials of immunotherapy drugs have demonstrated overall survival benefits in advanced or metastatic cancers (i.e. metastatic melanoma, non-small cell lung cancer, renal cancer and others). The majority of immunotherapy drugs are based on checkpoint blockade, blocking specific inhibitory interactions between immune system T cells and the cancer and/or dendritic cells (PD-1/PD-L1 interactions) or between T cells and dendritic cells (CLTA-4 interactions). Blocking these interactions allows T cells to grow, recognise and destroy the cancer. Combining immunotherapy's ability to suppress cancer cells inhibition of the T cells ability to recognise and destroy them and radiotherapy's ability to stimulate the tumour infiltration of T cells leads to synergistic effects during cancer treatment. The present invention increases cell apoptosis, release of immunostimulating signals and tumour infiltration of T cells and will consequently significantly enhance the synergistic effects of radiotherapy and immunotherapy.

Accordingly, in one embodiment, the invention provides a particle of the invention or a pharmaceutical composition of the invention for use in combination with radiotherapy in the treatment of cancer in a subject, wherein said treatment of the cancer further comprises immunotherapy. The particle and the treatment may be as further defined anywhere herein.

A pharmaceutical composition comprising a plurality of the nanoparticles is typically employed, and therefore the invention also provides a pharmaceutical composition of the invention for use in combination with radiotherapy in the treatment of cancer, wherein said treatment of the cancer further comprises immunotherapy. The pharmaceutical composition of the invention and the treatment may be as further defined anywhere herein. The pharmaceutical composition may further comprise an immunotherapeutic agent. The immunotherapeutic agent may be as further defined hereinbelow.

Immunotherapeutic agents that may be used in combination with the current invention include, but are not limited to, pembrolizumab, nivolumab, rituximab, ofatumumab, alemtuzumab, ipilumumab and atezolizumab. Immunotherapy may be performed before, during or after the radiotherapy. The immunotherapy generally comprises administering a immunotherapeutic agent to the subject. The immunotherapy may comprise administering an immunotherapeutic agent systemically, or administering a immunotherapeutic agent locally to a site of the cancer.

The invention also provides a method of treating cancer in a subject. The method generally comprises: administering to a subject a particle comprising a first semiconductor and a second semiconductor wherein the first semiconductor forms a heterojunction with the second semiconductor, and performing radiotherapy on the subject. Typically, the method comprises administering to a subject a pharmaceutical composition comprising a plurality of particles, wherein each of said particles comprises a first semiconductor and a second semiconductor wherein the first semiconductor forms a heterojunction with the second semiconductor, and performing radiotherapy on the subject.

The steps of administering to the subject a particle, or administering to the subject a pharmaceutical composition which comprises a plurality of the particles, and performing radiotherapy on the subject, may be as further defined anywhere herein. For instance they may be as further defined anywhere hereinbefore in the detailed description of the treatment of the invention.

In the method of treating cancer, administering to the subject said particle may comprise delivering said particle to a site of the cancer. Administering to the subject said pharmaceutical composition typically comprises delivering said plurality of particles to a site of the cancer.

Administering to the subject said particle or said pharmaceutical composition may comprise: introducing the particle or the pharmaceutical composition directly into a site of the cancer; or administering the particle or the pharmaceutical composition systemically and allowing the particle or particles to accumulate at a site of the cancer. As discussed in detail hereinbefore, introducing the particle or the pharmaceutical composition directly into said site of the cancer may for instance comprise injecting the particle or the pharmaceutical composition directly into said site of the cancer (e.g. intratumoral injection), or for example introducing the particle or the pharmaceutical composition directly into said site of the cancer via a catheter. The method may therefore comprise injecting said particle or said pharmaceutical composition, preferably injecting said particle or said pharmaceutical composition directly into the tumour. Administering the particle or the pharmaceutical composition systemically, on the other hand, usually comprises administering the particle or the pharmaceutical composition parenterally, for instance intravenously, intramuscularly or subcutaneously. Alternatively, it may comprise administering the particle or the pharmaceutical composition orally. Allowing the particle(s) to accumulate at the site of the cancer may comprise allowing the particle(s) to accumulate at said site by passive targeting or active targeting, as discussed in greater detail hereinbefore.

Administering to the subject said particle or said pharmaceutical composition may comprise administering the particle or pharmaceutical composition topically to a site of the cancer. A composition comprising the particles which is suitable for topical administration may be applied directly to a site of a cancer prior to radiotherapy. In such cases, the pharmaceutical composition comprising the particles suitable for topical administration may be in the form of a gel, cream, spray or paint. In particular, the site of the cancer may be a region of unresected tumour following surgery. In this case, the cancer may, for example, be a cancer of the bowel, colon, rectum or brain. Following tumour resection local reoccurrence is common and can be devastating since further surgery is often not indicated. Local reoccurrence is caused by small regions of unresected tumour remaining following surgery. A pharmaceutical composition in the form of a gel, cream, spray or paint can be used on the tumour bed following resection, prior to radiotherapy on the tumour bed. The composition will enhance the effectiveness of radiotherapy treatment of tumour beds and reduce local reoccurrence of the tumour. In this case, the particles may be labelled with active targeting to further enhance the uptake into tumour cells—the topical administration of the composition means that long circulation times in the blood supply are not required and active targeting is feasible. The pharmaceutical composition suitable for topical administration may comprise further ingredients such as water, alcohols, polyols, glycerol, vegetable oils, and the like; anti-oxidants, buffers, preservatives, stabilisers, bacteriostats, suspending agents, thickening agents, and solutes.

The method of treatment may further comprise detecting the presence or absence of the particle or pharmaceutical composition at a site of the cancer before performing radiotherapy. Typically, the step of detecting the presence or absence of the particle or particles at a site of a cancer comprises directing X-rays at the site to obtain an X-ray image. The X-ray image may then be used to determine if a cancer or tumour tissue is present or absent at the site and also whether the particle or pharmaceutical composition is present at the site. For diagnostic uses, the exposure time of a subject to X-rays is generally from one second to 30 minutes, typically from one minute to 20 minutes and more typically from one second to 5 minutes.

If the particle or particles comprises an optical contrast agent, a radioisotope, a paramagnetic contrast agent or a superparamagnetic contrast agent, then the agent may be used to perform the step of detecting the presence or absence of the particle or particles at the site. The exact method of detecting the particle or particles will depend on the optical contrast agent, radioisotope, paramagnetic contrast agent or superparamagnetic contrast agent that is present. The contrast agent may be a gadolinium MRI contrast agent.

The steps of performing radiotherapy on the subject, may be as further defined anywhere herein. For instance they may be as further defined anywhere hereinbefore in the detailed description of the treatment of the invention. Generally, the radiotherapy comprises irradiating a site of the cancer with radiation from an external source or from a radioactive material inside the subject.

In some cases, the method comprises delivering the particle (or particles) to a site of the cancer and irradiating the site of the cancer with radiation from an external source (external radiotherapy).

In other cases, when the method comprises irradiating a site of the cancer with radiation from a radioactive material inside the subject, the treatment typically further comprises administering the radioactive material to the subject. Administering the radioactive material to the subject may comprise: introducing the radioactive material directly into or near to said site of the cancer; or administering the radioactive material systemically and allowing the radioactive material to accumulate at said site of the cancer.

Typically, said site of the cancer comprises a tumour. Typically, the tumour comprises a hypoxic region. The hypoxic region may be as further defined hereinbefore.

In the method of the present invention, any type of cancer can, in principle, be treated. Thus, the method may for instance be used to treat a cancer of the lung, liver, kidney, bladder, breast, head and neck, oral cavity, throat, pharynx, oropharynx, oesophagus, brain, ovaries, cervix, prostate, intestine, colon, rectum, uterus, pancreas, eye, bone, bone marrow, lymphatic system, connective tissue, non-epithelial tissue or thyroid gland. The cancer may be prostate cancer, liver cancer, renal cancer, bone cancer, bladder cancer, cancer of the oral cavity, throat cancer, oropharyngeal cancer, sarcoma, lung cancer, cervical cancer, oesophageal cancer, breast cancer, brain cancer, ovarian cancer, intestinal cancer, bowel cancer, colon cancer, rectal cancer, uterine cancer, pancreatic cancer, eye cancer, lymphoma, or thyroid cancer. The bone cancer may be primary or metastatic. Typically, the method may be used to treat a cancer of the pancreas, head and neck, lung, bladder, breast, oesophagus, stomach, liver, salivary glands, kidney, prostate, cervix, ovaries, soft tissue sarcomas, melanoma, brain, bone or metastatic tumours arising from any primary tumour.

In some cases, the method may be used to treat a cancer of a radiosensitive organ. In such instances, the cancer may be a cancer of the salivary glands, liver, stomach, spinal column, lymph nodes, reproductive organs or digestive organs.

The treatment of the cancer may be multimodal. For instance, the method may be further combined with other treatments such as chemotherapy or immunotherapy, as further defined anywhere herein. The chemotherapy and immunotherapy may be as further defined anywhere hereinbefore in the detailed description of the treatment of the invention.

The invention also relates to an in vitro method of destroying cancer cells comprising contacting a particle comprising a first semiconductor and a second semiconductor wherein the first semiconductor forms a heterojunction with the second semiconductor with a composition comprising cancer cells, then directing ionising radiation at the cancer cells. In some cases, the invention relates to an in vitro method of destroying cancer cells comprising contacting a pharmaceutical composition comprising a plurality of said particles with a composition comprising cancer cells, then directing ionising radiation at the cancer cells.

The method of destroying cancer cells may comprise adding a particle or a pharmaceutical composition as described herein to a cell culture, medium or solution comprising cancer cells, then directing ionising radiation at the cancer cells. The ionising radiation typically comprises at least one selected from X-rays, gamma rays, protons, electrons (beta rays), positrons and alpha particles.

The invention also relates to a particle or a pharmaceutical composition for use in a diagnostic method practised on the human or animal body, typically for diagnosing the presence or absence of cancer.

Also provided is a method for determining the presence or absence of cancer comprising administering to a subject a particle or a pharmaceutical composition of the invention, then detecting the presence or absence of the particle or the pharmaceutical composition at a site suspected of being cancerous. The accumulation of the particles in a target tissue, whether by passive targeting or active targeting, may allow a tumour or cancer to be diagnosed by radiography, typically using conventional X-ray imaging methods. The presence of a heavy rare earth element in the particles that accumulate in the tumour may allow the tumour tissue to be visualised by X-rays.

Typically, the step of detecting the presence or absence of the particle or particles at a locus or site comprises directing X-rays at the locus or site to obtain an X-ray image. The X-ray image may then be used to determine if a cancer or tumour tissue is present or absent at the locus or site. For diagnostic uses, the exposure time of a subject to X-rays is generally from one second to 30 minutes, typically from one minute to 20 minutes and more typically from one second to 5 minutes.

If the particle or particles comprise an optical contrast agent, a radioisotope, a paramagnetic contrast agent or a superparamagnetic contrast agent, then the agent may be used to perform the step detecting the presence or absence of the particle or particles at the locus or site. The exact method of detecting the particle or particles will depend on the optical contrast agent, radioisotope, paramagnetic contrast agent or superparamagnetic contrast agent that is present. The contrast agent may be a gadolinium MRI contrast agent.

The invention further provides a kit of parts comprising: (i) a plurality of particles, wherein each of said particles comprises a first semiconductor and a second semiconductor, wherein the first semiconductor forms a heterojunction with the second semiconductor; and (ii) instructions for the use of the particles, in combination with radiation from an external source or from a radioactive material inside the subject, for the treatment of cancer in a subject.

Any of: the particles, the cancer to be treated, the subject to be treated, and the treatment by radiotherapy itself, may be as further defined anywhere herein.

The kit of parts of the invention may further comprise: a chemotherapeutic agent. The chemotherapeutic agent may be as further defined herein.

The kit of parts of the invention may further comprise an immunotherapeutic agent. The immunotherapeutic agent may be as further defined herein.

Where internal radiotherapy is employed, the instructions in the kit of parts are instructions for the use of the particles in combination with radiation from a radioactive material inside the subject, and the kit of parts may further comprise: a radioactive material suitable for internal radiation therapy. The radioactive material may be as further defined herein.

The present invention is further illustrated by the following Examples.

### EXAMPLES

### Example 1: Synthesis of Particles of the Invention and In Vitro Testing in Combination with X-Ray Radiotherapy

1. Synthesis of Particles Containing TiO₂ and Lu₂O₃ in a 0.91:0.09 Mass Ratio

130 g of 0.5 g/ml Dioctyl sulfosuccinate sodium salt (AOT) in isooctane is added to a beaker. To this is added a beaker containing 35 g of isooctane, 7 g of NaCl (at 2.5 g/100 ml), 44 g of 1-butanol and 65 g of DI water. This combined mixture is transferred to a round bottomed flask. To the round bottomed flask 2.5 ml of Titanium(IV) (triethanolaminato)isopropoxide solution is added at 30° C. and the solution is stirred for 1 hours. 1.5 ml of 0.4M Lu(NO₃)₃ is added and stirred. 0.33 ml of 1M NaOH is added and stirred. This solution is crystallised in a hydrothermal reactor at 170° C. for 1 hours prior to centrifugation and washing in isopropanol. The product is further crystallised by firing at 700° C. for fifteen minutes.

From this 50-60 nm particles of TiO₂ and Lu₂O₃ in a 0.91:0.09 mass ratio are produced. The mass ratio was established using energy dispersive X-ray spectroscopy (EDX). Electron micrograph (TEM) images of the particles are shown in FIG. 4.

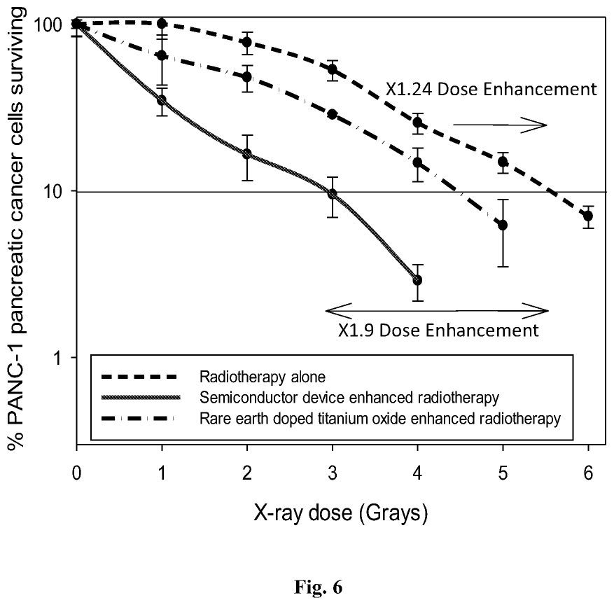

A comparison of EDX and X-ray photoelectron spectroscopy (XPS) results was used to ascertain that the second semiconductor (Lu₂O₃) exists as a separate phase on the surface of the first semiconductor (TiO₂). EDX was used for bulk composition measurements. XPS measures the top 1-10 nm composition. Therefore, the high XPS signals compared to EDX indicated a surface component. These results are shown in FIG. 5 for three samples with varying amounts of Lu₂O₃ deposited on the surface. Sample 5-C11 corresponds to the particles of TiO₂ and Lu₂O₃ in a 0.91:0.09 mass ratio prepared as described above. Sample 5-C12 corresponds to particles of TiO₂ and Lu₂O₃ in a mass ratio of 0.953:0.047 prepared by the same method as above but with 0.75 ml of 0.4M Lu(NO₃)₃. Sample 5-C13 corresponds to particles of TiO₂ and Lu₂O₃ in a mass ratio of 0.979:0.021 prepared by The same method as above but with 0.35 ml of 0.4M Lu(NO₃)₃.

As may be seen from the data, higher values are obtained for XPS measurements indicating that in all cases, the second semiconductor (Lu₂O₃) exists as separate phase on the surface of the first semiconductor (TiO₂).

### 2. Pancreatic Cancer (Panc-1) Clonogenic Survival Assay

Pancreatic cancer (Panc-1) cells were cultured in Dulbecco's Modified Eagle's Medium (DMEM) containing 10% foetal bovine serum, 2 mM L-glutamine and 50 μg/ml penicillin-streptomycin (culture media) at 37° C., 5% CO₂/95% air atmosphere with 95% relative humidity. Panc-1 cells were seeded in 6 well plates at 2000 cells per well and cultured overnight in the presence of 6.25 mg/ml (57 μM) of the particles as described above in part 1 of Example 1, and medium control well. Plates were then exposed to 0-6Gy X-ray radiotherapy and incubated for 6 days at 37° C., 5% CO₂. After 6 days plates were harvested and fixed, crystal violet stained and the number of colonies present determined by manual counting.

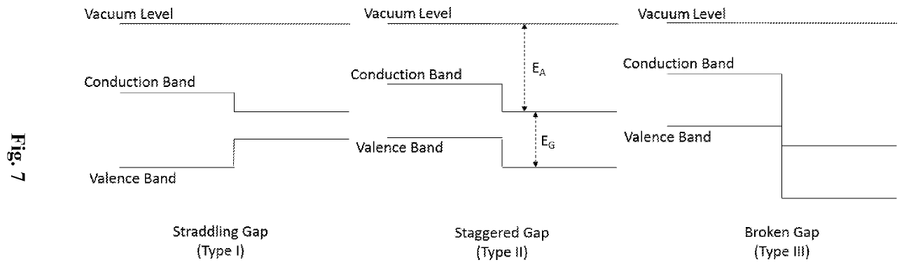

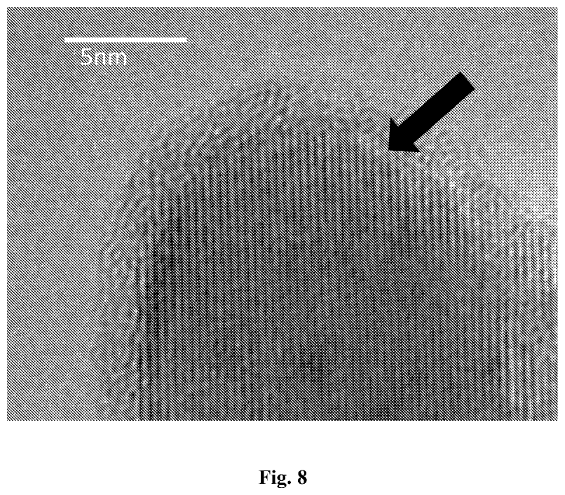

Results are presented in FIG. 6 as % survival against radiotherapy dose. Dose Enhancement Function (DEF) is defined as Radiotherapy dose/Radiotherapy dose+nanoparticles for an equivalent biological effect.

At 10% pancreatic cancer cell survival, DEF=1.9 for radiotherapy augmented with the particles described above in part 1 of Example 1. Also shown for comparison is the DEF for a rare earth doped titanium oxide nanoparticle such as those described in WO2011/070324. In this case the DEF is 1.24; a factor of 3.7 times lower than the DEF measured for particles of part 1 of Example 1 at an equivalent concentration per well.

### 3. Optional Addition of Silica Coating

A silica surface coating layer may be added to the particles of the present invention as follows. To 400 ml of ethanol, 97 ml deionised water and 12 ml ammonium hydroxide is added 0.25 g of particles as described above in part 1 of Example 1. Following sonication for 10 minutes, 2.43 of tetraethyl orthosilicate (TEOS) is added at 35° C. and the solution is stirred for 1 hour. The coated materials are then washed twice in isopropanol prior to dispersion in water and freeze drying.

### Example 2: Preparing an Injectable Pharmaceutical Formulation Comprising Particles of the Invention

An injectable solution of the particles comprising TiO₂ and Lu₂O₃ as prepared in Example 1 may be prepared as follows. 62.5 mg of sterile particles, prepared as described in part 1 of Example 1, are stored in a suitable sealed amber glass receptacle. Under clean room conditions the receptacle is opened and 10 ml of sterile filtered Dulbecco's phosphate buffered saline (Sigma-Aldrich) is added. Following addition, the dispersion of nanoparticles is agitated in an ultrasonic bath for 10 minutes prior to injection into the tumour.

### Example 3: Liver Cancer Treatment with Internal Radiotherapy (SIRT Yttrium-90 Beads) and Particles of the Invention

0.6 ml sterile glass 20-30 m microspheres containing beta emitting yttrium-90 (commercially available from BTG International under the trade name TheraSphere®) are dispersed in sterile water at a loading ranging from 0.2 mg·ml⁻¹ to 5 mg·ml⁻¹ for a typical single dose. Into this are dispersed particles of the invention as produced by Example 1, part 1. Loading of the particles of the invention is dependent on tumour volume, with a typical dose regime being 50 mg per 100 ml tumour volume. For a 5 cm diameter liver tumour, 32.5 mg of particles of the invention are added to the 0.6 ml water containing the yttrium-90 microspheres. Since the half-life of yttrium-90 is only 64.1 hours a set of six dose sizes are supplied ranging in activity from 3 GBq to 20 GBq. Becquerel (Bq) is the SI unit of radioactivity, 1 Bq being 1 decay per second, and a gigabecquerel (GBq) being 10⁹ becquerel. The target dose to the liver is 80-150 Gy which may be calculated using the formula:

$$Activity required ( GBq ) = \frac{[ Desired dose ( Gy ) ] [ Liver mass ( kg ) ]}{50}$$

A decay table supplied with the TheraSphere® microspheres allows the clinician to then calculate the appropriate time of injection to deliver the therapeutic formulation.

The formulation containing the particles of the invention comprising TiO₂ and Lu₂O₃ and the microspheres is then delivered to the patient using a catheter placed in the hepatic artery which supplies blood to the tumour. A catheter with an internal diameter of >0.5 mm is required to dispense the formulation within the artery. It is important that the catheter dose not occlude the blood vessel into which it is placed to avoid interruption of the blood flow responsible for dispersing the microspheres and nanoparticles within the tumour. The microspheres are unable to completely pass through the tumour vasculature due to capillary blockage and are trapped within the tumour. The particles of the invention, being <150 μm, travel through poorly aligned defective endothelial cells lining the tumour vasculature and preferentially accumulate within the tumour tissue, travelling through the extracellular matrix and undergoing endocytosis into the tumour cells. The particles of the invention disperse through the tumour and act to create hydroxyl free radicals by splitting water following interaction with electrons emitted by decaying yttrium-90.

### Example 4: Particles of the Invention with Internal Radiotherapy (Radium-223 Dichloride) Treatment of Castration Resistant Prostate Cancer (mCRPC) Bone Metastasis

Radium-223 and particles of the invention comprising TiO₂ and Lu₂O₃ as produced by Example 1, part 1, are delivered separately to treat mCRPC. Radium-223 dichloride is administered intravenously to a dose of 50 kBq per kg of body weight at a rate of one injection per week to a total of six injections.

Following suitable scanning, such as computed tomography (CT), of the bone tumour the optimum access point is determined and angle and distance to the tumour is calculated. Following the administration of local anaesthetic and a small skin incision a dedicated vertebroplasty bevelled needle (see “Osteoplasty: Percutaneous Bone Cement Injection beyond the Spine”; Anselmetti; Seminars in Interventional Radiology; Volume 27, Number 2, 2010, pp. 199-208) of 15-(1.372 mm internal diameter) or 10-(2.692 mm internal diameter) gauge diameter needle is advanced into the tumour. A bevel tip is preferred for ease of use and precise steering.

Once the CT indicates that the centre of the tumour has been reached by the needle tip a formulation of particles of the invention as produced by Example 1, part 1, dispersed in phosphate buffered saline (see Example 2) are injected into the tumour. A loading of 0.7 mg per ml tumour is required and the volume injected should be <10% of the total tumour volume. A large thigh bone tumour might have a diameter of 6 cm and a volume of 113 cm³. 70 mg of the particles of the invention are dispersed in 10 ml of phosphate buffered saline and injected directly into the centre of the tumour once a week for the duration of the Radium-223 treatment leading to a total injection of 420 mg of the particles of the invention over the course of the treatment. Typically, the particles of the invention would be injected into different points of the tumour at each injection to ensure maximum distribution of the particles throughout the tumour volume.

Radium-223 substitutes for calcium in the bone whereas the particles of the invention disperse through the tumour via the extracellular matrix and are taken up into the cell. Properties of the bone matrix, including low oxygen content and acidic pH create an environment favourable for tumour growth but unfavourable for treatment using oxygen based free radicals. The particles of the invention enhance the treatment of mCRPC bone metastases by generating cell-killing hydroxyl free radicals by splitting water following Radium-223 alpha particle scattering.

### Example 5: Synthesis of Particles Containing TiO₂ and Lu₂O₃ in a 0.91:0.09 Mass Ratio

4 g of titanium dioxide powder was dispersed in 200 mL of deionised water at 25° C. An aqueous solution of 0.5M lutetium nitrate was added dropwise. An aqueous solution of 0.2M potassium hydroxide was added dropwise to raise the pH to 6-8. The dispersion was washed and the particles recovered by centrifugation and freeze dried. The dried powder was fired in a furnace at a temperature of 750° C. degrees for approximately 5 minutes to produce the nanoparticles.

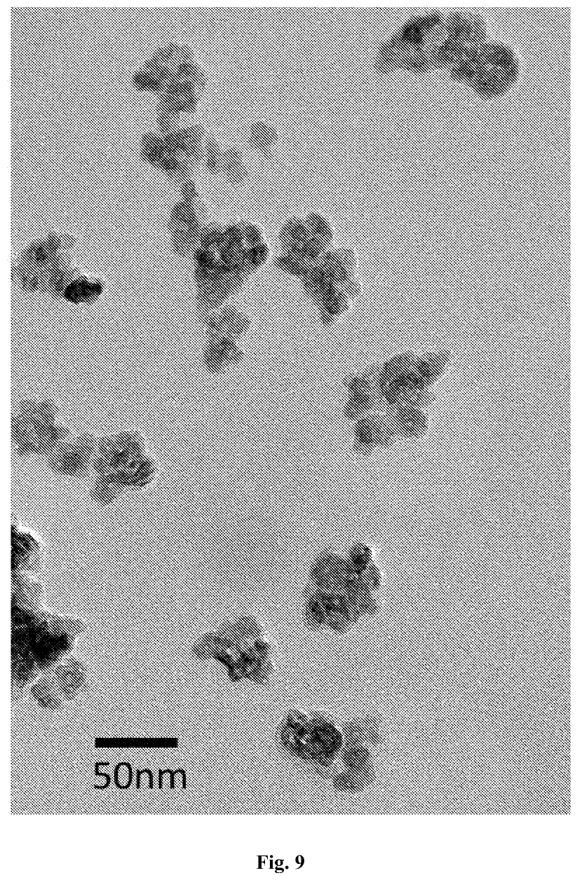

From this particles of TiO₂ and Lu₂O₃ in a 0.91:0.09 mass ratio with an average particle size of approximately 50 nm were produced. A transmission electron micrograph of these particles is shown in FIG. 9.

### Example 6: Synthesis of Particles Containing TiO₂ and Gd₂O₃ in a 0.93:0.07 Mass Ratio

4 g of titanium dioxide powder was dispersed in 200 mL of deionised water at 25° C. An aqueous solution of 0.5M gadolinium nitrate was added dropwise. An aqueous solution of 0.2M potassium hydroxide was added drop wise to raise the pH to 6-8. The dispersion was washed and the particles recovered by centrifugation and freeze dried. The dried powder was fired in a furnace at a temperature between 750° C. degrees to produce the nanoparticles.

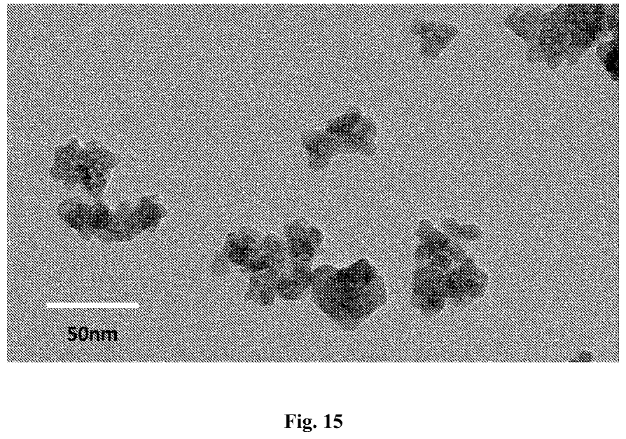

From this particles of TiO₂ and Gd₂O₃ in a 0.93:0.07 mass ratio with an average particle size of 50 nm were produced. A transmission electron micrograph of these particles is shown in FIG. 14.

### Example 7: Synthesis of Particles Containing TiO₂ and Yb₂O₃ in a 0.93:0.07 Mass Ratio

4 g of titanium dioxide powder was dispersed in 200 mL of deionised water at 25° C. An aqueous solution of 0.5M ytterbium nitrate was added dropwise. An aqueous solution of 0.2M potassium hydroxide was added drop wise to raise the pH to 6-8. The dispersion was washed and the particles recovered by centrifugation and freeze dried. The dried powder was fired in a furnace at a temperature between 750° C. degrees to produce nanoparticles of (TiO₂)_{0.91}(Yb₂O₃)_{0.9}

From this particles of TiO₂ and Yb₂O₃ in a 0.93:0.07 mass ratio with an average particle size of 50 nm were produced. A transmission electron micrograph of these particles is shown in FIG. 15.

### Example 8: Clonogenic Assay of Particles Produced in Examples 5-7

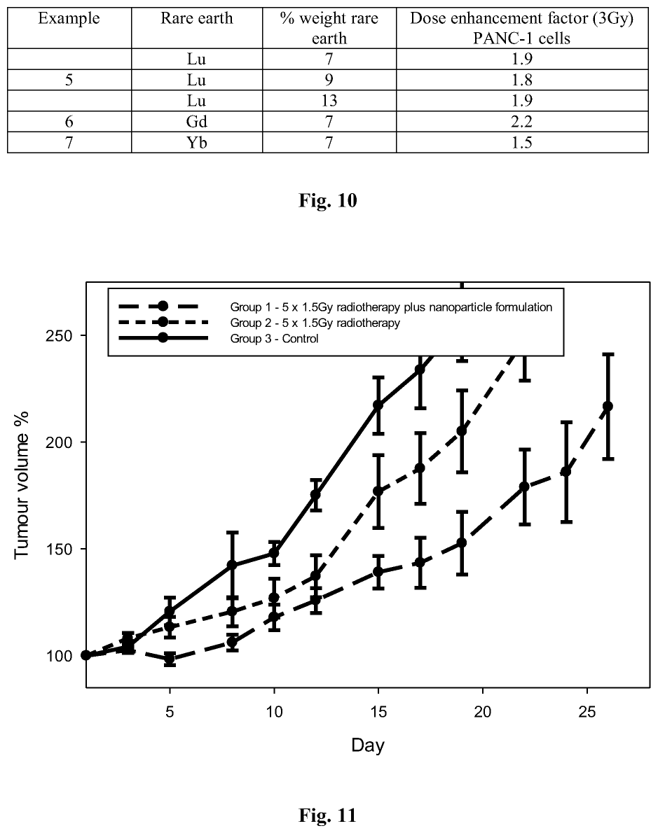

The Panc-1 pancreatic adenocarcinoma human cell line was thawed and expanded to provide sufficient cells for the assay. Pancreatic cancer (Panc-1) cells were cultured in Dulbecco's Modified Eagle's Medium (DMEM) containing 10% foetal bovine serum, 2 mM L-glutamine and 50 g/ml penicillin-streptomycin (culture media) at 37° C., 5% CO₂/95% air atmosphere with 95% relative humidity prior to harvesting and seeded into 6-well plates. Cells were seeded at 2,000 cells per well, with particle formulations, and media only control, each run in triplicate with n=3 wells. Nanoparticles were added at 6.25 mg per well. Plates were cultured for 24 hrs prior to irradiation at 0 and 3 Gy doses. The plates were incubated at 37° C., 5% CO₂. After 6 days plates were harvested and fixed, Crystal Violet stained and the number of colonies present determined by manual counting. The relative survival of colonies at 0 and 3 Gy and control and nanoparticle treated wells was used to calculate the enhancement of cell killing, the radiotherapy dose enhancement factor (DEF). The results are given in FIG. 10 for particles produced by examples 5, 6, 7. In addition results from particles produced using example 5 with different weight % loading of lutetium are included.

### Example 9: Preparing an Injectable Pharmaceutical Formulation Comprising Particles of the Invention

An injectable solution of the particles of TiO₂ and Lu₂O₃ in a 0.91:0.09 mass ratio as prepared in Example 5 was prepared as follows. 50 mg of sterile particles, prepared as described in Example 5, were stored in a suitable sealed glass receptacle. Under clean room conditions the receptacle was opened and 2 ml of sterile filtered 5% glucose (B Braun Petzold) was added. Following addition, the dispersion of nanoparticles was agitated in an ultrasonic bath for 10 minutes prior to injection into the tumour. Typically, particles were dosed at a volume equivalent to 5 or 10% of the total tumour volume.

### Example 10: Treatment of Pancreatic Cancer Xenografts Using a Pharmaceutical Formulation Comprising Particles

An injectable pharmaceutical formulation comprising the particles of TiO₂ and Lu₂O₃ in a 0.91:0.09 mass ratio was prepared as described in Example 9. In order to assess the therapeutic effect of the formulation prepared in Example 9 a study was undertaken in which the formulation was combined with radiation therapy in the human pancreatic cancer xenograft model Mia-Paca2 in male CD-1 nude mice. Male CD-1 nude mice were purchased at 4-6 weeks of age and were held in individually ventilated cages (IVCs) in an SPF barrier unit. All procedures are certified according to the UK Home Office Animals (Scientific Procedures) Act 1986. Mice were housed for 1-2 weeks prior to use to stabilise the animals. Animals are xenografted on one flank with MiaPaca2 cell line, the tumours being left to grow until mean tumour volume reaches approximately 200 mm³. Mice were then randomised into 3 groups of n=12 per group and were treated as follows.
Group 1-Irradiation 5×1.5Gy for 5 days plus nanoparticle formulation as described in Example 9 administered once on day 1 via intra-tumoral injection.
Group 2-Irradiation 5×1.5Gy for 5 days
Group 3-No treatment control group.
   Tumours were measured with callipers 3× weekly once the xenografts reach approximately 100 mm³ and the animals were weighed 3× weekly. The endpoint of the study was the number of days until the tumour volume doubled in size. The results are shown in FIG. 11. The time to tumour volume doubling is 13.7 days in the case of the control (Group 3), 18.3 days for radiotherapy alone (Group 2) and 25.0 Days for radiotherapy plus nanoparticle formulation (Group 3). This demonstrates that nanoparticle enhanced radiotherapy is 2.5 times as effective as radiotherapy alone in controlling Mia-PaCa2 pancreatic tumour xenografts.

### Example 11: Optional Coating of Polyvinylpyrrolidone (PVP)

Nanoparticles produced by any preceding example are added to a suitable diluent, for example glucose or DI water. PVP powder is added at a ratio of 2:1 weight nanoparticles to PVP producing a dispersion of particles functionalised with a PVP coating which may be freeze dried and have a negative surface charge.

### Example 12: Optional Coating of Sodium Hexametaphosphate (HEX)

Nanoparticles produced by any preceding example are added to a suitable diluent, for example an aqueous solution of glucose (sterile filtered 5% glucose; B Braun Petzold) or DI water. Sodium hexametaphosphate powder is added at a ratio of 2:1 weight nanoparticles to sodium hexametaphosphate producing a dispersion of particles functionalised with a phosphate polymer coating which may be freeze dried and have a negative surface charge.

### Example 13: Treatment of Colorectal Cancer Xenografts Using a Pharmaceutical Formulation Comprising Particles

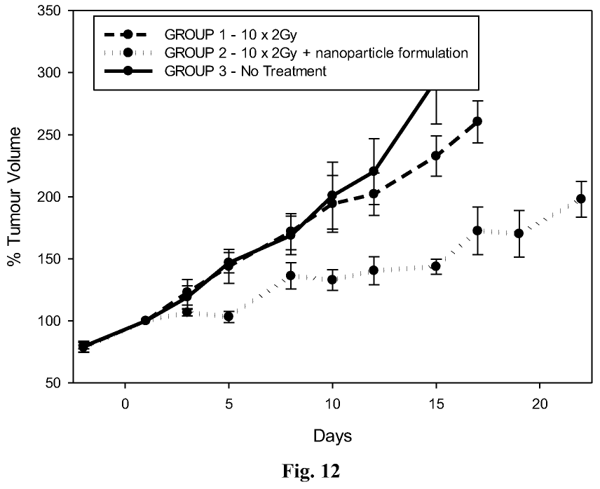

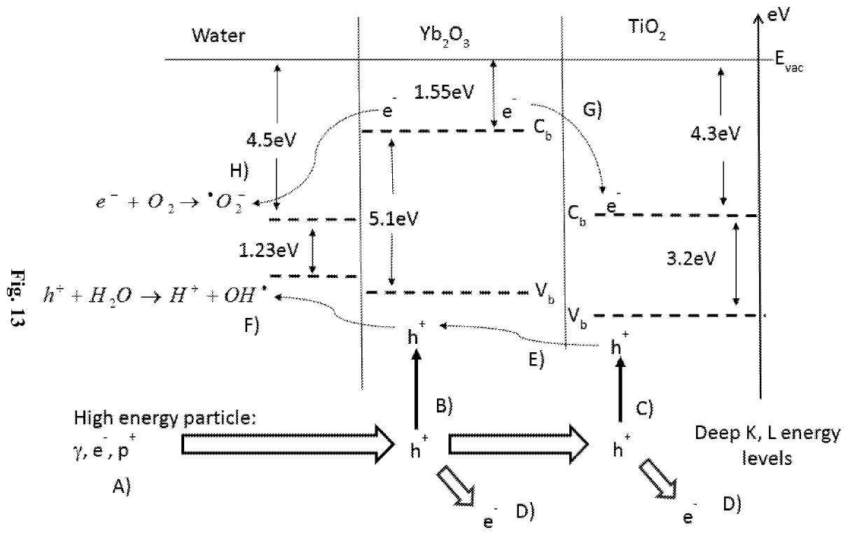

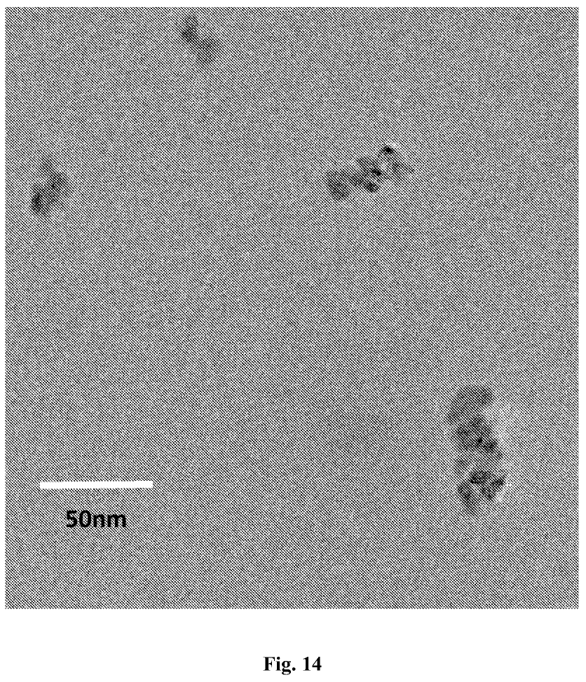

An injectable pharmaceutical formulation comprising particles of TiO₂ and Lu₂O₃ in a 0.91:0.09 mass ratio, in sterile filtered 5% glucose solution, was prepared as described in Example 12. The nanoparticles were produced in accordance with Example 5. In order to assess the therapeutic effect of the formulation prepared in Example 12 a study was undertaken in which the formulation was combined with radiation therapy in a radioresistant human colorectal cancer xenograft model in male CD-1 nude mice. Male CD-1 nude mice were purchased at 4-6 weeks of age and are held in individually ventilated cages (IVCs) in an SPF barrier unit. All procedures were certified according to the UK Home Office Animals (Scientific Procedures) Act 1986. Mice were housed for 1-2 weeks prior to use to stabilise the animals. Animals were xenografted on one flank with the radioresistant colorectal cell line, the tumours being left to grow until mean tumour volume reaches approximately 150 mm³. Mice were then randomised into 3 groups of n=6 per group and were treated as follows.
Group 1-Irradiation 10×2 Gy over 2 cycles of 5 days.
Group 2-Irradiation 10×2 Gy over 2 cycles of 5 days plus nanoparticle formulation as described in Example 12 administered once on day 1 via intra-tumour injection.
Group 3-No treatment control group.
   Tumours were measured with callipers 3× weekly once the xenografts reached approximately 100 mm³ and the animals were weighed 3× weekly. The endpoint of the study was the number of days until the tumour volume doubled in size. The results are shown in FIG. 12. The time to tumour volume doubling is 9.9 days in the case of the control (Group 3), 11.4 days for radiotherapy alone (Group 1) and 22.0 Days for radiotherapy plus nanoparticle formulation (Group 2). This demonstrates that nanoparticle enhanced radiotherapy is 8.1 times as effective as radiotherapy alone in controlling radioresistant colorectal tumour xenografts.

### Example 14: Particles of the Invention with Internal Radiotherapy (Permanent Implant Brachytherapy) for the Treatment of Localised Prostate Cancer

At least one week prior to implantation of the brachytherapy seeds a transrectal ultrasound examination of the prostate is undertaken and volume of the gland and tumour ascertained. Iodine-125 brachytherapy seeds (0.8 mm×4.5 mm titanium capsules containing Iodine-125 as silver iodide within a porous ceramic and a gold X-ray marker) with an activity of 0.015 GBq per seed are implanted to a total dose to the planned target volume of 145 Gy. The seeds are implanted into the prostate gland using needles passing through the perineum and are guided into position by ultrasound analysis. Typically, 70 to 150 seeds are implanted.

Iodine-125 decays by electron capture, a process by which a proton-rich nucleus stabilises by capturing an inner shell electron forming a neutron and a neutrino. Iodine-125 decays to an excited state of tellurium-125 which subsequently emits gamma rays as it stabilises to ground state tellurium-125. These gamma rays have energy emissions of 27.4, 31.4, 35.5 keV. In order to convert this energy to hydroxyl radicals to treat hypoxic tumour regions the titanium oxide particles comprise a second semiconductor containing an element whose K-edge energy most closely matches the emission energy of the Iodine-125 seeds.

Iodine-125 has a half-life of 60 days. Particles of the invention comprising titanium dioxide partially coated with ruthenium oxide and/or molybdenum oxide (which may be prepared using the method of Example 1, for example by employing ruthenium (III) chloride and/or molybdenum (III) chloride as the starting materials instead of the Lu(NO₃)₃) are injected into the prostate tumour following seed insertion once swelling has decreased sufficiently. A typical male prostate is 15-30 ml in size which may be >75% tumour. For a tumour of 20 ml volume a 2 ml dispersion of 35 mg·ml⁻¹ particles of the invention are injected into the tumour directly in one, or more, injections dispersed in phosphate buffered saline. This may be repeated a second time 30 days into treatment if necessary. The particles of the invention directly absorb the gamma rays emitted by Iodine-125 generating hydroxyl free radicals from the nanoparticle surface and treating hypoxic regions of the tumour.

1. A particle comprising a first semiconductor and a second semiconductor wherein the first semiconductor forms a heterojunction with the second semiconductor.

2-100. (canceled)
